(19) United States
(12) Patent Application Publication
(10) Pub. No.: US 2010/0196439 A1
(43) Pub. Date: Aug. 5, 2010

(54) Angiogenesis Mechanism and Method, and Implantable Device

(75) Inventors: Michael Theodore Beck, Eden Prairie, MN (US); Deon Bezuidenhout, Cape Town (ZA); Kelvin Bonnema, Brooklyn Park, MN (US); Kimberly A. Chaffin, Woodbury, MN (US); Neil Davies, Vredehoek (ZA); Brian C.A. Fernandes, Roseville, MN (US); Eric J. Fogt, Maple Grove, MN (US); Eric A. Grovender, Minneapolis, MN (US); Robert Cushing Hamlen, Edina, MN (US); Matthew Jolly, Minneapolis, MN (US); Eamonn J. McAndrew, County Galway (IE); Syamasundar V. Pakala, Blaine, MN (US); William P. VanAntwerp, Valencia, CA (US); Peter P. Zilla, Cape Town (ZA); Edouard A. Koullick, Golden Valley, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/520,035

(22) PCT Filed: Dec. 20, 2007
(86) PCT No.: PCT/US07/88407

§ 371 Date: Apr. 21, 2010

Related U.S. Application Data
(60) Provisional application No. 60/876,532, filed on Dec. 22, 2006.
(60) Provisional application No. 60/890,041, filed on Feb. 15, 2007.
(60) Provisional application No. 60/890,326, filed on Feb. 16, 2007.

(51) Int. Cl. A61K 9/00; A61K 38/18; A61K 35/12; A61P 3/10

(52) U.S. Cl. 424/424; 514/12; 424/93.1

(57) ABSTRACT

Various methods and devices are provided for providing therapy to a living body. In one embodiment, an implanted permeable container has living cells provided therein, and various nutrients and/or agents are provided into the container to promote cell life.

### CROSS-REFERENCE TO RELATED APPLICATIONS

The present application claims the benefit of priority from PCT Application Serial No. PCT/US07/088,407, published as WO 2008/079997, filed Dec. 20, 2007, which claims priority to U.S. Provisional Application Ser. No. 60/876,532, filed Dec. 22, 2006, U.S. Provisional Application Ser. No. 60/890,041, filed Feb. 15, 2007 and U.S. Provisional Application Ser. No. 60/890,326, filed Feb. 16, 2007, the entire contents of which are all incorporated herein by reference.

### FIELD OF THE INVENTION

The present invention relates to therapeutic implantable medical devices and, more particularly, to implantable cell therapy devices that facilitate improved cell viability.

### BACKGROUND OF THE INVENTION

Type I diabetes (formerly known as insulin-dependent diabetes, childhood diabetes, or juvenile-onset diabetes) is most commonly diagnosed in children and adolescents, but can occur in adults, as well. It is characterized by β-cell destruction, which usually leads to an absolute deficiency of insulin. Most cases of Type I diabetes are immune-mediated, characterized by autoimmune destruction of the body's β-cells in the islets of Langerhans of the pancreas, destroying them or damaging them sufficiently to reduce insulin production. However, some forms of Type I diabetes are characterized by loss of the body's β-cells without evidence of autoimmunity.

Currently, Type I diabetes can be treated only with insulin, with careful monitoring of blood glucose levels using blood testing monitors. Emphasis is also placed on lifestyle adjustments (diet and exercise). Apart from the common subcutaneous injections, it is also possible to deliver insulin via a pump, which allows infusion of insulin of up to 24 hours a day, and the ability to program a push dose (a bolus) of insulin as needed at meal times. This is at the expense of an indwelling subcutaneous catheter. Type I treatment by introduction of insulin must be continued indefinitely.

Another method of treatment that has been proposed is via a pancreas transplant. Pancreas transplants, however, have been met with limited success because introducing a new, functioning pancreas to a patient with diabetes can have negative effects on the patient's normally functioning kidney. Additionally, the availability of pancreases is limited because an organ donor is necessary.

Direct islet cell transplantation has also been proposed as a treatment for Type I diabetes. In this procedure, islet cells (e.g., porcine or human islet cells) are injected directly into the patient's liver, where the cells take residence and begin to produce insulin. The liver is chosen because it is more accessible than the pancreas and the islet cells seem to produce insulin well in that environment. The patient's body, however, may treat the new cells just as it would any other introduced foreign tissue. Specifically, the immune system may attack the cells as it would a viral infection. Thus, the patient also may need to undergo treatment involving immunosuppressants, which typically have a systemic effect and reduce immune system activity.

Type I diabetes is an example of a condition for which implanted therapeutic devices offer a possibility of successful treatment. Numerous other conditions exist that could be treated upon the development of an implanted medical device that overcomes the shortcomings of the prior art.

The performance of an implanted medical device in a recipient can be affected by factors that are not related to the function of the device. For example, cells within a living cell containing device may not be provided with a sufficient blood supply from the recipient, such that the device may perform poorly. In addition, the device may also perform poorly due to a lack of nutrients or oxygen provided to the cells. Alternately, the device may perform poorly due to an accumulation of waste products in or around the device. In addition, implanted devices are sometime rejected by a process known as the “foreign body response” wherein inflammatory cells, in particular macrophages, perceive the surface of the implanted device as a foreign or non-natural material and initiate a cascade of cellular and tissue reactions, leading to a physiological state of chronic inflammation and fibrous encapsulation of the implanted device. Fibrous capsules surrounding an implanted device can prevent communication and exchange between the device and the surrounding tissue. Various approaches to vascularization of implanted devices have been reported.

It is an object of the present invention to provide various devices and methods of treatment that overcome some of the shortcomings associated with the prior art.

### SUMMARY OF THE INVENTION

In accordance with an embodiment of the present invention, an implantable device for providing therapy to a living body is provided that includes a permeable container and therapeutic living cells carried by the container. The living cells are capable of generating cell products that are emitted from the permeable container to the living body. A conduit associated with the permeable container provides at least one agent to the living cells carried by the container and in an exemplary embodiment, a pump operatively connected with the conduit is configured to pump the at least one agent through the conduit to the living cells.

In accordance with another embodiment of the present invention, an implantable device for providing therapy to a living body is provided that includes a permeable container and therapeutic living cells carried by the container. The living cells are capable of generating cell products that are emitted from the permeable container to the living body. A void space within the container receives agents that enhance living conditions for the living cells.

In accordance with another embodiment of the present invention, a method is provided for providing therapy to a living body that includes implanting a permeable container in the living body and delivering growth factors to the implanted permeable container to facilitate vessel formation on the permeable container. Therapeutic living cells are introduced to the implanted permeable container and at least one agent is provided to the therapeutic living cells carried by the implanted permeable container.

In accordance with another embodiment of the present invention, a method is provided for providing an in vivo vascularized medical implant device including implanting a permeable container in a living body and delivering growth factors to the implanted permeable container to facilitate vessel formation on the permeable container. Living therapeutic cells are implanted in the implanted permeable container after the vessels have been formed on the permeable container and a pump is implanted in communication with the therapeutic cells that outputs at least one agent to the cells to promote cell life.

In accordance with another embodiment of the present invention, a method is provided for providing an in vivo vascularized medical device that includes implanting a container in a living body that has an exterior wall portion and an interior wall portion. The exterior wall portion is permeable to materials of a greater size than said interior wall portion. Growth factors are delivered to the implanted container, facilitating vessel formation on the exterior wall portion and substantially preventing formation of vessels within the confines defined by the interior wall portion. Growth factors may be delivered by elution from device as part of polymer eluting system contained in a device or surfaces coated with growth factors, e.g., elution from container surfaces coated with growth factors. Growth factors may be delivered by a pump configured to pump growth factors to the container.

In accordance with another embodiment of the present invention, an assembly for providing a vascularized, implanted device, is provided that includes an implantable container having an exterior wall portion and an interior wall portion. The exterior wall portion is permeable to materials of a greater size than said interior wall portion. The assembly further includes a conduit associated with the implantable container and a pump configured to pump growth factors to the container. The interior wall portion enables the growth factors to pass therethrough to reach the exterior wall portion to facilitate vessel formation on the exterior wall portion. The interior wall portion prevents vessel formation within confines defined thereby.

In accordance with another embodiment of the present invention, an assembly for providing a vascularized, implanted device, is provided that includes an implantable permeable container and a removable barrier disposed within the container that is permeable to growth factors and impermeable to vessels. The assembly further includes a conduit in communication with the permeable container and a pump connected with the conduit and configured to pump growth factors through the conduit and into the container. The growth factors permeate through the barrier and to the permeable container to facilitate vessel formation on the permeable container.

In accordance with another embodiment of the present invention, a method of providing a vascularized, implanted therapeutic device is provided that includes implanting a permeable container having an interior wall surface in a living body and inserting a formation of encapsulated therapeutic cells within the implanted permeable container. The formation of therapeutic cells is expanded into contact with the interior wall surface of the implanted permeable container.

In accordance with another embodiment of the present invention, an assembly for providing an implanted therapeutic device is provided that includes a permeable container and a formation of underhydrated encapsulated therapeutic living cells. The assembly further includes an insertion device for inserting the formation of underhydrated encapsulated therapeutic living cells into the permeable container.

In accordance with another embodiment of the present invention, a formation of underhydrated encapsulated therapeutic living cells is provided that includes a mass of underhydrated hydrogel and a plurality of islet cells within the underhydrated hydrogel.

In accordance with another embodiment of the present invention, an assembly for providing an implanted therapeutic device is provided that includes a permeable container having an interior wall surface and a hydrogel carrier having a shield structure formed therein. The hydrogel carrier and the shield structure are constructed to fit within the permeable container. The assembly further includes a formation of hydrogel encapsulated therapeutic cells disposed on the hydrogel carrier. The carrier is movable into the container so as to position the therapeutic cells adjacent to the interior wall surface of the permeable container and the shield structure protects the formation of encapsulated therapeutic cells during transport thereof to said position adjacent the interior wall surface.

In accordance with another embodiment of the present invention, a method is provided for vascularizing an implanted device that includes implanting a container comprising a porous outer surface, an interior cavity, and a permeable cell barrier that separates the outer surface from the interior cavity and delivering a growth factor composition comprising, for example, autologous platelet gel serum (APGS) or vascular endothelial growth factor (VEGF), to the implanted container for a period of time sufficient to induce formation of new blood vessels. A growth factor composition may be formed as part of polymer system contained in a device or coated on one or more surfaces of the device, such that at least one growth factor is eluted into fluid moving through the device and delivered to tissues surrounding the device. A growth factor composition may be introduced into fluid moving through the device. At least some of the new blood vessels are in direct contact with the outer surface of, and/or within the walls of, the implanted container.

In accordance with another embodiment of the present invention, an implanted device is provided that includes a porous container and an interior cavity within the porous container. The device further includes a permeable cell barrier that separates the porous container from the interior cavity, a pump in communication with the interior cavity, and a growth factor composition comprising APGS provided within the pump, and being pumped into the interior cavity within the porous container.

In accordance with another embodiment of the present invention, a method of providing an implantable device is provided that includes withdrawing a sample of blood from a patient and obtaining APGS from the blood of the patient. A permeable container is implanted into the patient. The permeable container has an exterior surface portion and an interior surface portion, the interior surface portion defining a cavity within the container, the exterior surface portion having a greater permeability than the interior surface portion. The APGS is introduced into the cavity in the implanted permeable container, and migrates through the interior surface portion to the exterior surface portion to facilitate blood vessel formation on and/or within the exterior surface portion. The interior surface portion substantially prevents ingress of blood vessels into the cavity so as to inhibit blood vessel formation within the cavity.

In accordance with another embodiment of the present invention, an implantable device for providing therapy to a living body is provided that includes a permeable container and therapeutic living cells carried by the container. A conduit extends into the permeable container and in communication with the therapeutic living cells and an oxygen pump connects with the conduit for delivering oxygen to the therapeutic living cells.

In accordance with another embodiment of the present invention, a method of providing therapy to a patient is provided that includes implanting a porous container into a patient and pumping a growth factor into the container to promote vessel formation on an outer surface of the porous container. After the vessel formation, an encapsulated construct of therapeutic cells is implanted into the container and at least one agent is pumped into the container to promote life of the therapeutic cells. After a period of time, the construct of therapeutic cells is removed from the porous container without removing the porous container from the patient and a new construct of encapsulated cells is replaced into the porous container.

In accordance with another embodiment of the present invention, an implantable container is provided comprising a permeable interior wall portion, an exterior wall portion permeable to materials of a greater size than the interior wall portion, and the interior wall portion enabling growth factors to pass therethrough to reach the exterior wall portion to facilitate vessel formation on the exterior wall portion, the interior wall portion preventing substantial vessel formation within confines defined thereby.

In accordance with another embodiment of the present invention, an implantable device for providing therapy to a living body is provided, comprising a permeable container, therepeutic living cells carried by the container, the living cells capable of generating cell products that are emitted from the permeable container to the living body, and an agent source is in communication with the permeable container for providing at least one agent to the living cells carried by the container.

In accordance with another embodiment of the present invention, an assembly for providing a vascularized, implanted device is provided, comprising an implantable container having an exterior wall portion and an interior wall portion, the exterior wall portion being permeable to materials of a greater size than said interior wall portion, a source connected with the implantable container that provides growth factors to the container, and the interior wall portion enabling the growth factors to pass therethrough to reach the exterior wall portion to facilitate vessel formation on the exterior wall portion, the interior wall portion preventing substantial vessel formation within confines defined thereby.

In accordance with another embodiment of the present invention, an assembly for providing a vasularized, implanted device is provided, comprising an implantable permeable container, a removable barrier disposed within the container that is permeable to growth factors and impermeable to vessels, and an agent source in communication with the permeable container and configured to provide growth factors into the container, the growth factors permeating through the barrier and to the permeable container to facilitate vessel formation on the permeable container.

In accordance with another embodiment of the present invention, an implanted device is provided comprising (a) a porous container, (b) an interior cavity within the porous container, (c) a permeable cell barrier that separates the porous container from the interior cavity, (d) an agent source in communication with the interior cavity and providing a growth factor composition comprising APGS to the interior cavity within the porous container.

In accordance with another embodiment of the present invention, a method of providing therapy to a patient is provided, comprising implanting a porous container into a patient, providing a growth factor into the container to promote vessel formation on an outer surface of the porous container, after the vessel formation, implanting an encapsulated construct of therapeutic cells into the container, providing at least one agent into the container to promote life of the therapeutic cells, after a period of time, removing the construct of therapeutic cells from the porous container without removing the porous container from the patient, and, after the removing, replacing a new construct of encapsulated cells into the porous container.

In accordance with another embodiment of the present invention, an assembly for providing a vascularized, implanted device, is provided comprising an implantable container formed from a permeable material, a barrier structure disposed within the container and formed from a permeable material, the barrier structure being permeable to materials of a smaller size in comparison to the implantable container, an agent source arranged to provide growth factors to the barrier structure, the growth factors permeable through the barrier structure and through the implantable container, and the barrier structure constructed to prevent substantial vessel formation therein.

In accordance with another embodiment of the present invention, an implantable device for providing therapy to a living body is provided, comprising a container permeable to growth factors, and an agent source for providing at least one agent to the container, where the agent source is arranged to be in communication with the container through a conduit. In one exemplary embodiment, a surface of one or more of the agent source, the container, or the conduit is coated with a growth factor composition that is eluted from the surface and emitted to the body. In another exemplary embodiment, the agent source comprises growth factor composition formed as part of a separate polymer system that can be contained in the device, wherein the polymer system may be a polymer plug, cartridge, capsule, hydrogel, or other polymer matrix than can contain growth factor composition and from which at least one growth factor can be eluted and emitted to the body. In one embodiment, the agent source comprises a growth factor composition comprising VEGF coated on the surface or contained within a polymer matrix, and VEGF is eluted from the surface and emitted to the body. In another embodiment, the surface of the agent source is coated with APGS and at least one growth factor is eluted from the surface and emitted to the body. The surface may also be coated with an agent that enhances the effectiveness of another agent, or an agent that affects the environment in which the delivery device and agents are found, e.g., an anticoagulant such as heparin. The device may further comprise therapeutic living cells carried by the container, where the living are cells capable of generating cell products that are emitted from the permeable container to the living body. The agent source may provide at least one agent to the living cells carried by the container, wherein the agent enhances living conditions for cells, e.g., various nutrients, immunosuppressive agents, growth factors, or other cell-life-promoting agents.

In accordance with another embodiment, an implantable device for providing therapy to a living body, comprising a container permeable to growth factors, an agent source for providing at least one agent to the container, wherein the agent source is arranged to be in communication with the container through a conduit, wherein a fluid stream passes through the container, the agent source, and the conduit, and further comprises a separate polymer system in contact with the fluid stream, wherein the polymer system comprises a growth factor composition formed as part of the polymer system, so as to allow at least one growth factor to be eluted into the fluid stream, wherein the polymer system is placed in the device so to be in contact with the fluid stream. In an exemplary embodiment, the polymer system that can be contained in the device, wherein the polymer system may be a polymer plug, cartridge, capsule, hydrogel, or other polymer matrix than can contain growth factor composition and from which at least one growth factor can be eluted and emitted to the body. In one embodiment, at least one growth factor is eluted from the polymer system and emitted to the body. In one embodiment, the polymer system comprises a growth factor composition comprising VEGF, and VEGF is eluted from the polymer system and emitted to the body. In another embodiment, the polymer system comprises APGS and at least one growth factor is eluted from the polymer system and emitted to the body. The device may further comprise therapeutic living cells carried by the container, wherein the living cells are capable of generating cell products that are emitted from the permeable container to the living body, and the agent source may provides at least one agent to the living cells carried by the container, wherein the agent enhances living conditions for cells.

Other objects, features, and advantages of the present invention will become apparent from the following detailed description, the accompanying drawings, and the appended claims.

### BRIEF DESCRIPTION OF THE DRAWINGS

The above-mentioned and other features and advantages of the present invention, and the manner of attaining them, will become more apparent and the disclosure itself will be better understood by reference to the following description taken in conjunction with the accompanying drawings, wherein:

FIG. 1 is a schematic representation illustrating a general process of cell therapy in accordance with an embodiment of the present invention;

FIG. 2 is a schematic perspective view, partly in section, illustrating showing a vascularized container in accordance with an embodiment of the present invention;

FIG. 3 is a schematic perspective view, partly in section, illustrating a vascularized container in accordance with another embodiment of the present invention;

FIG. 4 is a schematic perspective view of a trapezoidal cell container and cell barrier assembly in accordance with another embodiment of the present invention;

FIG. 4A is a schematic perspective view of a trapezoidal cell container and hydrogel construct in accordance with another embodiment of the present invention;

FIG. 5 is a schematic perspective view of various elements of a cell therapy device in accordance with an embodiment of the present invention;

FIG. 6 is an exploded perspective view of elements in a vascularization assembly in accordance with an embodiment of the present invention;

FIG. 7 is a perspective cross-sectional view of an assembled vascularization assembly in accordance with an embodiment of the present invention;

FIG. 8 is a cross-sectional view of a container assembly in accordance with an embodiment of the present invention;

FIG. 8A is a cross-sectional view of a container assembly in accordance with another embodiment of the present invention;

FIG. 9 is a cross-sectional view of a cell barrier assembly in accordance with an embodiment of the present invention;

FIG. 9A is a cross-sectional view of a cell barrier assembly in accordance with another embodiment of the present invention;

FIG. 10 is a cross-sectional view of a vascularization assembly with pump in accordance with an embodiment of the present invention;

FIG. 10A is a cross-sectional view of a vascularization assembly with pump in accordance with another embodiment of the present invention;

FIG. 10B are two comparative cross-sectional views of two vascularization assemblies in accordance with two embodiments of the present invention;

FIG. 10C is a cross-sectional view of a vascularization assembly with pump and venting system in accordance with an embodiment of the present invention;

FIG. 11 is a cross-sectional view of a vascularized container assembly in accordance with an embodiment of the present invention;

FIGS. 12A and 12B show various steps and apparatuses used to form a hydrogel in accordance with an embodiment of the present invention;

FIG. 12C shows various steps and apparatuses used to form a hydrogel in accordance with another embodiment of the present invention;

FIG. 12D is a cross-sectional view of a PEG-based hydrogel construct being inserted into a permeable container assembly in accordance with an embodiment of the present invention;

FIG. 13 is a cross-sectional view illustrating a step of the insertion of a PEG-based hydrogel into a container assembly in accordance with an embodiment of the present invention;

FIG. 14 is a cross-sectional view illustrating a further step of the insertion of a PEG-based hydrogel construct into a container assembly in accordance with an embodiment of the present invention;

FIG. 15 is a cross-sectional view of a cell therapy device with a PEG-based hydrogel construct in accordance with an embodiment of the present invention;

FIG. 16 is a cross-sectional view showing a step in the preparation of an agarose-based hydrogel construct in accordance with an embodiment of the present invention;

FIG. 16A is a cross-sectional view of an agarose-based hydrogel construct prior to insertion into a container assembly in accordance with an embodiment of the present invention;

FIG. 17 is a cross-sectional view of a cell therapy device with an agarose-based hydrogel construct in accordance with an embodiment of the present invention;

FIG. 18 is a cross-sectional view of an alternative agarose-based hydrogel assembly in accordance with an embodiment of the present invention;

FIG. 19 is a cross-sectional view of a cell therapy device with an agarose-based hydrogel construct with an end cap in accordance with an embodiment of the present invention;

FIG. 20 is a cross-sectional view of a cell therapy device having two separate supply lines in accordance with an embodiment of the present invention;

FIG. 21 is a schematic diagram of an oxygen supply system in accordance with an embodiment of the present invention;

FIG. 22 is a perspective view of an electro-chemical gas generating cell in accordance with an embodiment of the present invention;

FIG. 23 is a graph showing partial pressure of oxygen across a catheter wall and islet/alginate matrix thickness in accordance with an embodiment of the present invention;

FIG. 24 is a graph showing critical volumetric cell packing density as a function of islet/hydrogel matrix thickness in accordance with an embodiment of the present invention;

FIG. 25 is a graph showing oxygen consumption per islet as a function of islet packing density in accordance with an embodiment of the present invention;

FIGS. 26-31 show steps in preparing an alginate hydrogel matrix over a permeable catheter and insertion into a container in accordance with an embodiment of the present invention;

FIG. 32 is a perspective view of a cell therapy device with wire springs in accordance with an embodiment of the present invention;

FIG. 33 shows the chemical structure for a polyethylene glycol-based hydrogel in accordance with an embodiment of the present invention;

FIG. 34 is an exploded perspective view of a vascularization assembly in accordance with an alternative embodiment of the present invention;

FIG. 35 is a graph showing dissolved oxygen as a function of time in accordance with an alternative embodiment of the present invention;

FIGS. 36-49 show various steps in the manufacture of a cell therapy device using a flat sheet type container in accordance with another embodiment of the present invention;

FIGS. 50A and 50B show insertion tools for use in a cell therapy process in accordance with an embodiment of the present invention; and

FIGS. 51A-51C show retaining clips for use in a cell therapy process in accordance with an embodiment of the present invention.

FIGS. 52A-52C illustrate a molding technique that may be used in the present inventions.

FIGS. 53A-53F illustrate a method of inserting a flat sheet hydrogen into a container in accordance with one embodiment of the invention.

FIGS. 54A-54E show various flat container assemblies and container plugs that can be used in accordance with an embodiment of the present invention.

The present invention will be described with reference to the accompanying drawings. Corresponding reference characters indicate corresponding or similar parts throughout the several views. The description as set out herein illustrates an arrangement of the invention and is not to be construed as limiting the scope of the disclosure in any manner.

### DETAILED DESCRIPTION OF THE INVENTION

In accordance with certain embodiments of the present invention, methods, apparatuses, and compositions are provided for improving the viability of implanted cells or other biological materials used in cell therapy or other therapeutic processes. Cell therapy processes for treating Type I diabetes mellitus by transplantation of pancreas islet cells is primarily discussed herein, but one of ordinary skill in the art will appreciate that the methods and apparatuses disclosed may have a wide variety of applications, such as, for example, cardiac cell therapy, hemophilia cell therapy, enzymatic defect cell therapy, synthetic vascular grafts, viral drug delivery, sensor technology, ischemic repair, wound healing, markers for therapy for Type I or Type II diabetes, artificial organ or cell transplants, hormone therapy, hepatitis treatment, or for the treatment of neural disease states such as Alzheimer's, Parkinson's or Huntington's diseases. Thus, the present disclosure should not be construed as being limited to any specific application disclosed, but includes the full scope of any patentable combination of elements or features disclosed herein, as well as the subject matter recited in the appended claims and their equivalents.

### Overview:

The cell therapy processes according to certain embodiments of the present invention can generally be broken down into three areas: (a) pre-vascularization of a cell container, (b) loading of cells into the container to form a cell therapy device, and (c) maintenance of the cell therapy device.

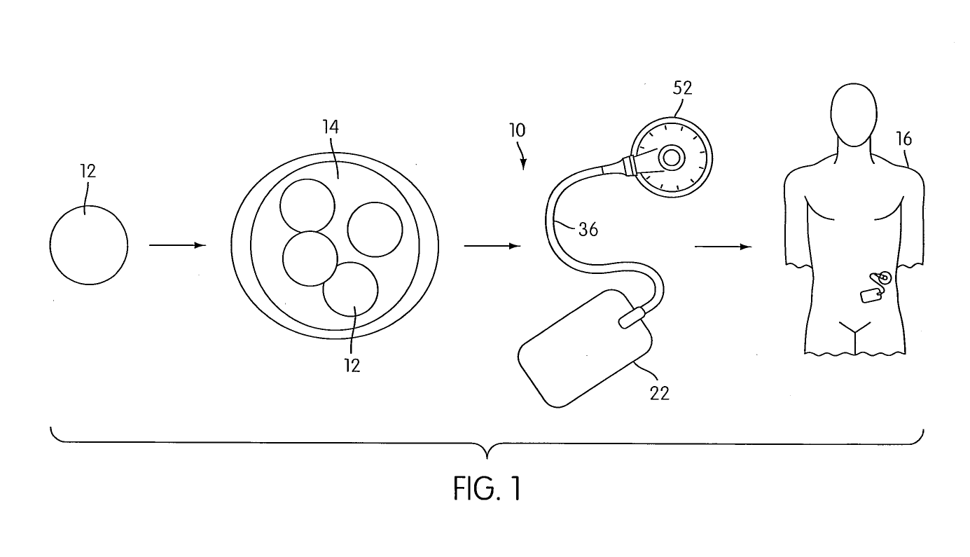

FIG. 1 shows a general concept of one embodiment of the invention. In this embodiment, therapeutic cells 12, such as porcine or human islet cells, are macroencapsulated in a gel construct 14. Other therapeutic agents or cells 12 that may be used in accordance with the invention for various therapies may include thyroid or parathyroid cells, cells producing nerve growth factors, adrenal cells, cells producing Factor XIII or Factor IX, cells producing interferon, or hepatocytes. Additional therapeutic cells that may be used in accordance with the present invention may also include cells with high angiogenic potential, including, but not limited to adipose derived stromal cells, mononuclear cells from blood, and bone marrow derived cells. The cells 12 may alternatively or in addition be microencapsulated and are retained in a cell therapy device 10. In one embodiment, the device 10 includes a cell container 22, pump 52, and catheter 36. The device 10 is then implanted in a patient 16 for treatment.

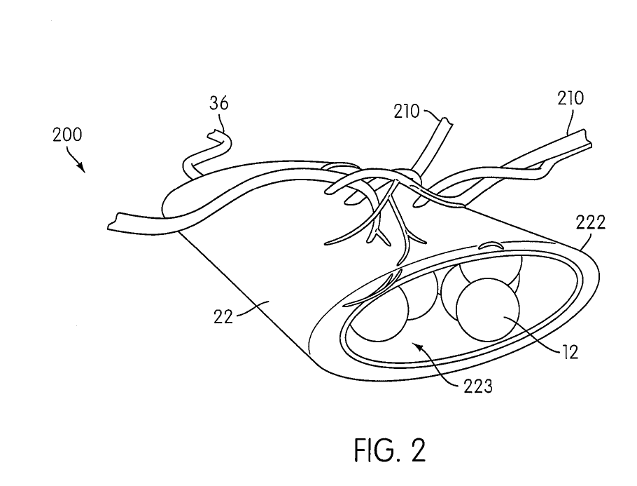

As shown generally in FIG. 2, the container 22 may be formed from a permeable or porous material 222.

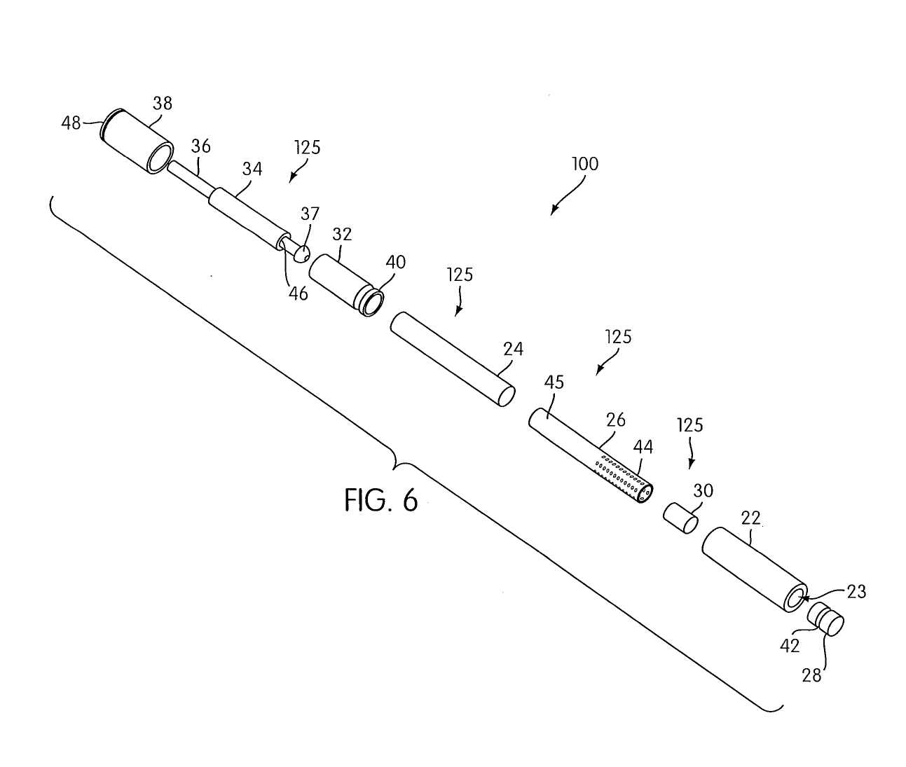

The container 22 in one embodiment is tubular in shape (e.g., see hollow cylindrical container 22 in FIGS. 6-8) or may take the form of any shaped pouch, bag, sack, or other configuration or structure that may contain, hold, or carry materials. In the examples of FIGS. 2, 3, and 4, the container 22 is shown as a generally flat pouch, sheet, bag, or a trapezoidal construct.

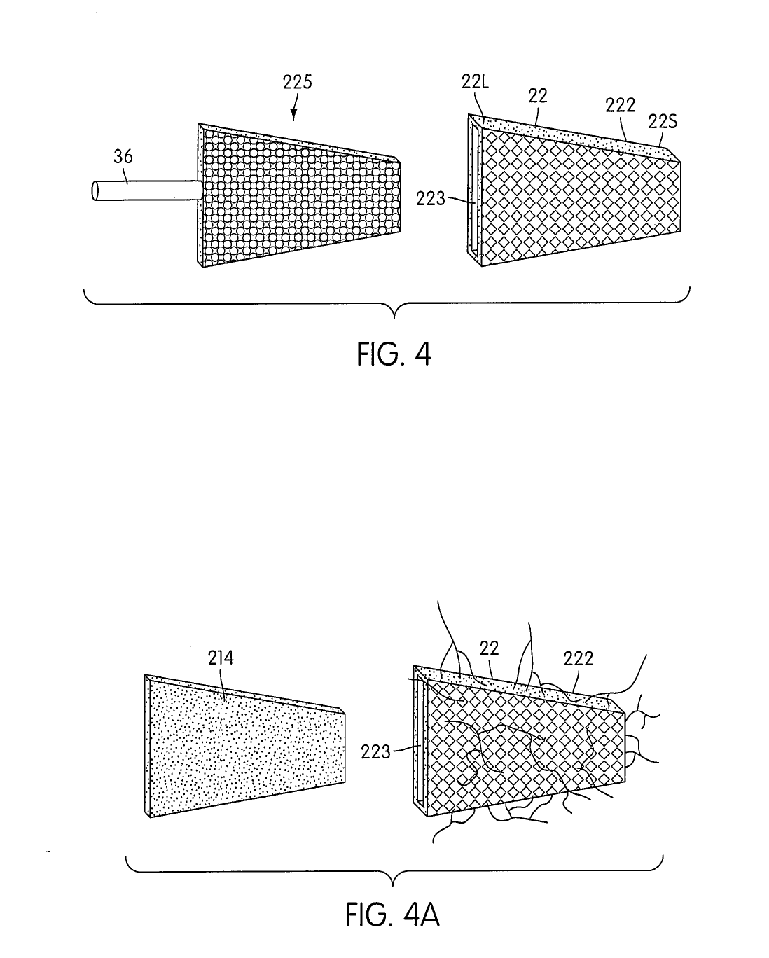

The embodiment of FIG. 4 shows a trapezoidal container 22 and an associated trapezoidal cell barrier assembly 225. The trapezoidal porous (or permeable) container 22 has a generally corresponding trapezoidal shaped internal cavity, and an opening 223 at the larger end 22L of the container 22 for receiving the cell barrier assembly 225. The smaller end 22S of the trapezoidal container 22 may close off the pocket-like opening 223. In this embodiment, the cell barrier assembly 225 may be formed from a block of permeable or porous material. In one embodiment, the permeable or porous material may be a sintered material, such as sintered metal or sintered polymer, for example. This will be described in more detail later.

In an embodiment, the cell barrier assembly 225 may include a heat-sealed pocket or pouch made from a porous polyvinylidene difluoride (PVDF) membrane and a non-woven insert. The non-woven insert may be substantially or entirely contained within the pouch. One or more catheters 36, or pump tubes, may be connected to the non-woven insert at the larger end (base) of the cell barrier assembly 225.

In an embodiment, the assembly 225 may be connected to two or more pumps so that the pump rate of the drug/growth factor may be increased. In an embodiment, particularly for larger assemblies, the catheters may be routed to different locations within the cell barrier assembly 225 to improve distribution of the drug/growth factor being delivered to the cell barrier assembly 225.

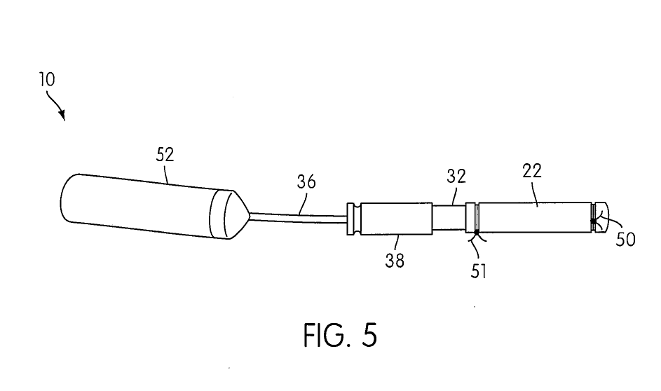

FIG. 5 shows an assembled cell therapy device 10, which generally includes a pump 52, a catheter 36, an end cap 38, a feeder tube 32, and a cell container 22 with attachment sutures 50, 51.

The term “container” is used herein to broadly refer to any structure capable of containing or carrying a therapeutic agent. In one embodiment, the present invention contemplates that the container herein may be formed from a permeable or porous material, which material may be manufactured in accordance with the teachings of U.S. Pat. Nos. 6,616,699 and 6,702,848, both to Zilla et al., the entire contents of both being hereby incorporated by reference in their entirety.

The term “pump” is used herein to broadly refer to any structure for supplying at least one agent to container 22. In addition, as disclosed in non-limiting embodiments herein, the present invention contemplates the use of pumps, as well as devices that do not utilize a pump. It can thus be appreciated that any mechanism for providing agents to container 22 may be employed.

It should be appreciated that the term “permeable” as used herein is intended to broadly refer not only to materials that may allow fluids to diffuse therethrough, but also to porous materials and materials with holes therethrough.

As can be appreciated from FIGS. 6, 7, and 8, and as discussed above, in one embodiment, the cell container 22 is a hollow structure formed of porous polyurethane material. However, the container 22 may, in another embodiment, be formed of polyethylene terephthalate (PET) or other appropriate material. The porous container material has interconnecting pores such that they provide small passages through the thickness of the container 22. Such pores communicate an inner space or cavity 23 within the container 22 with the surrounding flesh, blood cells, and blood vessels surrounding the outer surface of the implanted container 22. In one embodiment, the container 22 has a majority of the pores with diameters in the range of about 30 to 250 microns, and in one embodiment between 90 and 200 microns, and in one embodiment the pore diameters average about 130 microns, and in another embodiment, the pore diameters average about 165 microns.

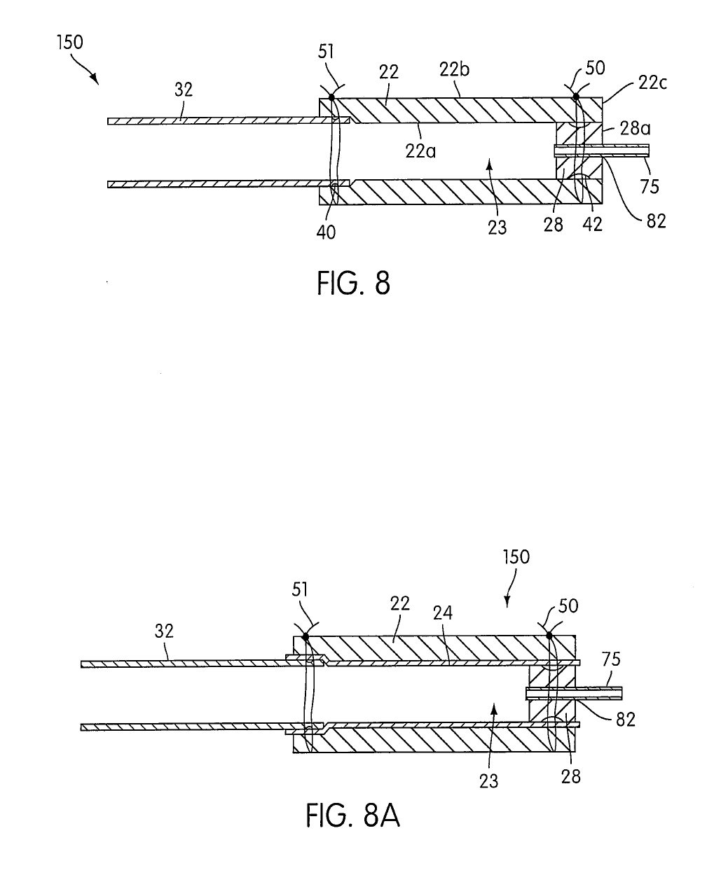

In one configuration, as can be seen in FIG. 8, the tubular configuration of the container 22 is substantially cylindrical, having a cylindrical interior wall 22a and a cylindrical exterior wall 22b. In this configuration, the cavity 23 has a form conducive to receiving pumped agents. In one embodiment, the thickness of the container material, between the walls 22a and 22b, is between about 200 microns and 1500 microns, and in one embodiment between about 300 microns and 600 microns.

Other shapes, wall thicknesses, and pore sizes of the container 22 may be desired depending on the specific application or therapy being applied. A flat pouch or envelope type container 22, as illustrated in FIGS. 2-4, can provide an additional benefit of being able to position cells contained therein in closer proximity to the surrounding vasculature and vessels surrounding the container 22. Specifically, a generally flat container provides a generally flat space 223 therein, with two opposing, closely spaced inner container surfaces having access to vasculature surrounding the container. Thus a thin, sheet-like cell containing gel construct inserted in the space 223 will have access to (and be in close proximity with) blood vessels on both sides of the container exterior, thereby increasing the surface area in contact with or in close operative proximity to vasculature by as much as two times or more as compared to a tubular cell construct. In contrast, a tubular or cylindrical configuration for a cell carrying construct may tend to limit such direct or close access to the vasculature to essentially the outer cylindrical layer of implanted cells or materials that are in contact with the container interior wall 22a.

Optionally, and as discussed in greater detail later, angiogenesis may be promoted with the aid of vascular growth factors to vascularize the container 22 by forming blood vessels 210 into and around its porous body (see FIG. 2).

In a following step, as will be described, a therapeutic agent, such as the aforementioned living islet cells, is loaded into the cell container 22 once the container 22 is sufficiently vascularized. The cells 12 may be encapsulated in the aforementioned gel construct 14. For example, the containment material 14 may take the form of a polyethylene glycol-(PEG) based hydrogel construct (see chemical structure in FIG. 33), an agarose-based construct, or an alginate-based construct. Constructs may also be based on such materials as chitosan, collagen, or other suitable materials. These containment materials may protect against possible immune attack by direct cell contact (primary immune response) and/or facilitate the transfer of nutrients, metabolites, and desired cell products (e.g., insulin and glucose). Various other processes and structures may be utilized as desired to optimize the transplantation of cells and their transport qualities. Exemplary non-limiting processes and structures are described herein.

Various steps may be taken to aid in keeping the implanted cells 12 alive and well nourished. For example, various nutrients, immunosuppressive agents, growth factors, or other cell-life promoting agents, may be supplied or fed to the islet cells. Also, further provisions are described for maintaining an adequate supply of oxygen to the transplanted cells 12.

It should also be appreciated that it may be desirable to periodically remove the implanted cells 12 and replace them with new cells. One aspect of the present invention provides for the retention of the developed vasculature 210 in and around the cell container 22 while the encapsulated cells 12 are replaced.

Various devices, processes and methods of manufacture in accordance with certain aspects of the invention will now be described in greater detail.

A. The Vascularization Assembly

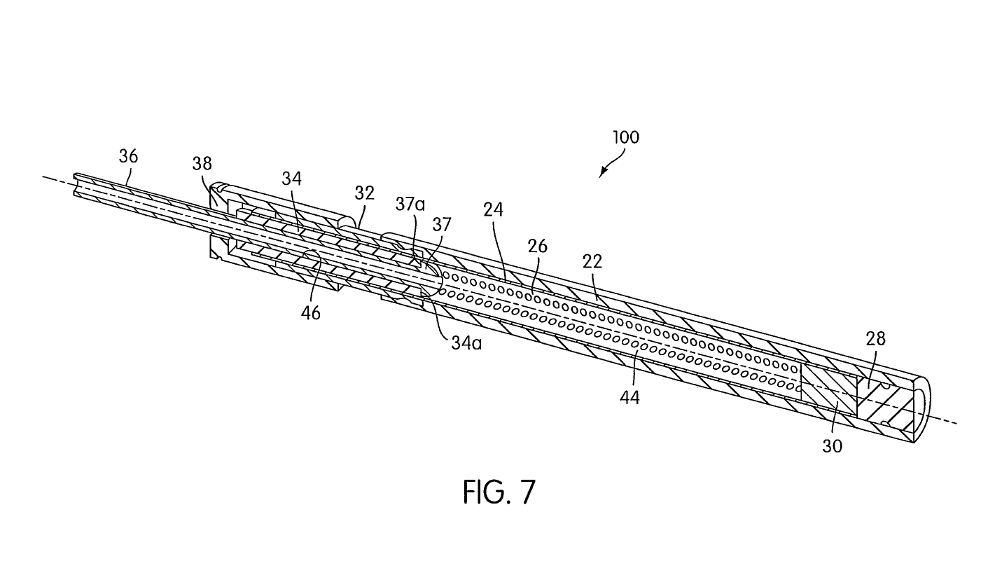

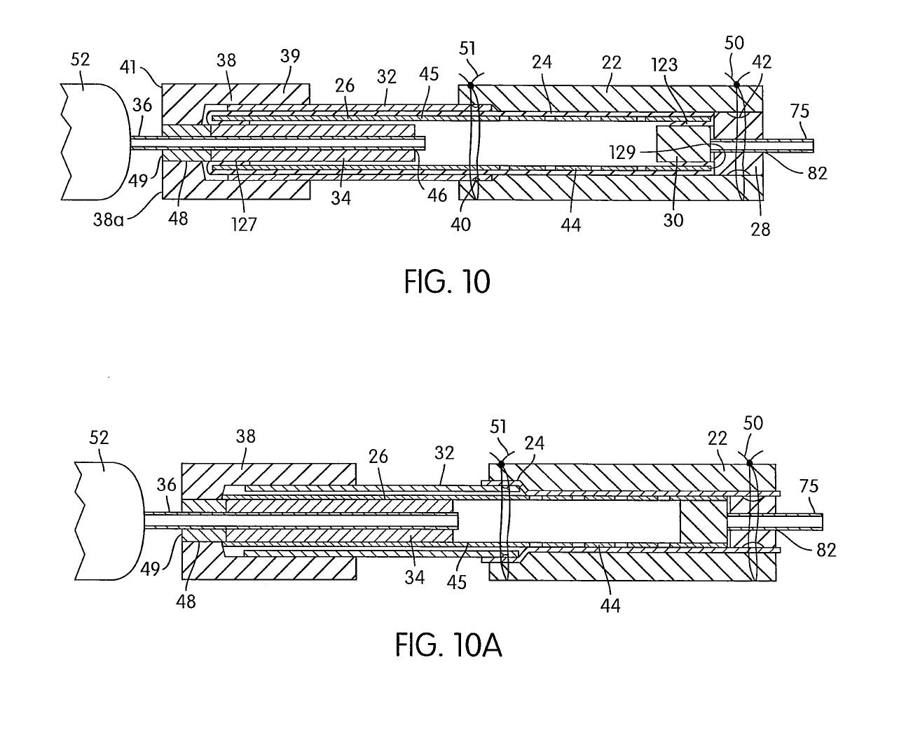

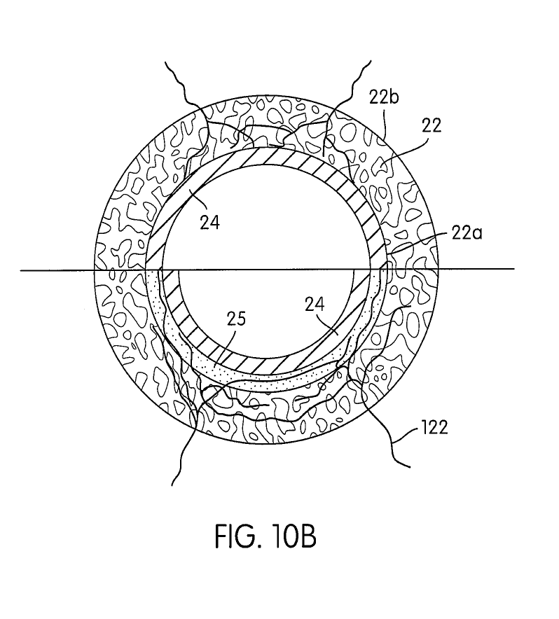

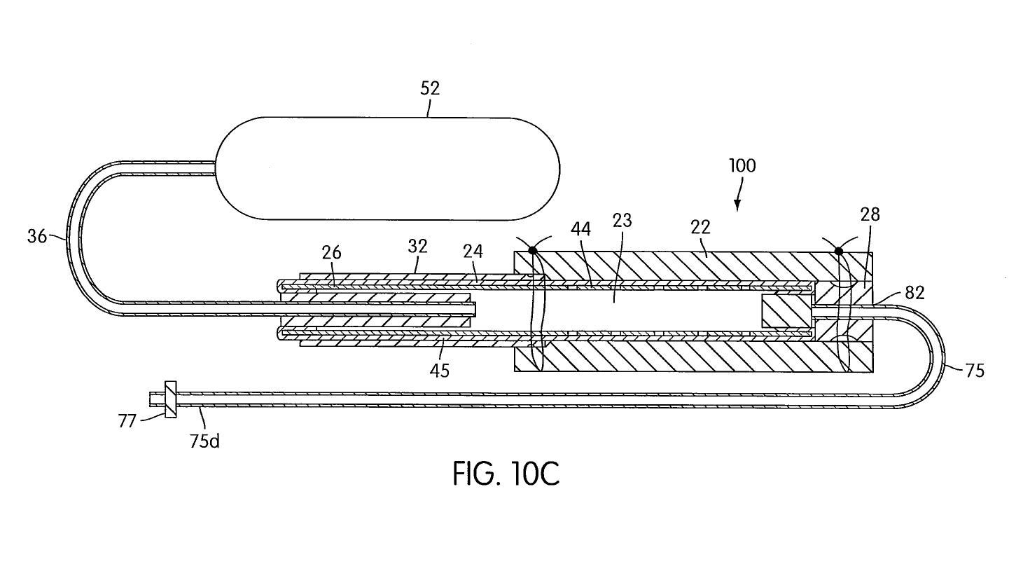

In one embodiment of the present invention, a vascularization assembly 100, as illustrated in FIGS. 7, 10, and the exploded view of FIG. 6, is assembled prior to being implanted into a patient. The assembly 100 includes, among other things, the aforementioned permeable cell container 22. In the embodiment illustrated in FIGS. 6, 7, and 10, a tubular, or hollow cylindrical container is used. The vascularization assembly 100 may be implanted subcutaneously but other locations may be suitable for implantation, such as the intraperitoneal cavity or wall, an intramuscular site, an abdominal fat pad, or another suitable location. The primary purpose of the implantation of the vascularization assembly 100 at this point is to promote angiogenesis in and around the container 22 such that, upon subsequent loading or transplantation of therapeutic cells 12 into the container 22, vasculature or blood vessels may be readily available for transport of materials (i.e., cell products, insulin, glucose, nutrients, etc.) between the transplanted cells 12 and the circulatory system, and to supply oxygen to the cells 12.

Another attribute of vascularization assembly 100 is (while promoting vascularization through and around the pores of the container 22) that it may be used to limit the development of blood vessels such that they do not form to a significant extent within an inner space or lumen 23 within the container 22 or such that the blood vessels form to a specifically controlled extent. Specifically, in the event that space 23 within container 22 becomes filled with blood vessels through angiogenesis, it may subsequently be more difficult to load islet cells into the space 23, or may reduce the number of islet cells that can be loaded into container 22. Accordingly, the vascularization assembly 100 is provided with a cell barrier 24, as will be discussed in greater detail later.

As illustrated in FIGS. 6-10, in addition to container 22 and cell barrier 24, the vascularization assembly 100 may further include a barrier support 26, a support plug 30, a feeder tube 32, a cored plug 34, a pump 52 and a pump connection line 36 (e.g., a catheter), an end plug 28, and an end cap 38 (see exploded view, FIG. 6). One of skill in the art will appreciate that one or several of these members may be omitted as the application may dictate, or that additional hardware may be included. Such omissions and/or additions remain within the scope and spirit of the present invention.

It should also be appreciated that the same or different pumps may be used to a) pump materials or agents (such as growth factors) that promote angiogenesis or the container 22, and b) pump materials or agents that support islet cell life after islet cells (or other therapeutic cells) are implanted in the container. Thus, such pumping mechanisms are generically labelled with reference numeral 52, although it should be understood that different pumps can be used. Similarly, catheter 36 may be the same or different types, sizes, construction, and/or materials of catheters may be used at the different stages of processes described herein, but are generically labelled with reference 36 in the figures. Similarly, it can be appreciated that container 22 may take several different forms, materials, and/or constructions, but is generically labelled by reference numeral 22 for convenience herein.

To assemble the vascularization assembly 100, the tubular cell container 22 may first be prepared by securing an end plug 28 into one end thereof and a feeder tube 32 into an opposite end thereof (see FIG. 8). The end plug 28 may be inserted into a distal end of the container 22 so that a distal end surface 28a of the end plug 28 and the distal end surface 22c of the container 22 are flush or nearly flush. The end plug 28 may be held in place by sutures 50 that are secured around the outer circumferential surface 22b of the container 22 to thereby exert inward force around the outer circumferential surface 22b, so that the inner surface 22a is forced into engagement with the outer surface of the end plug 28.

In one embodiment, the end plug 28 may have a bore 82 for insertion of a venting tube 75. In such an embodiment, one end of the venting tube 75 is open to the inner lumen 23 of the cell container 22 while the other end may be exposed in open air (for example, during surgery). The venting tube 75 therefore allows objects to be inserted into and removed from the container 22 without realizing air pocket or vacuum effects within the cell container 22. The bore 82 and venting tube 75 are only one form of venting the cell container 22, and numerous other ways of venting can be readily appreciated by those skilled in the art.

The container 22 may be sufficiently flexible to deform locally upon tightening of the sutures 50, and the end plug 28 may have an annular groove 42 around its outer circumferential surface so that the deformed portion of the container 22 is pressed into the groove 42 in a secure manner. Of course, other mechanisms for securing the end plug 28 may be used, such as an adhesive connection. In yet another embodiment, the end plug 28 can be eliminated and substituted by a porous polyurethane wall portion that is integrally formed with and is essentially a continuation of the material forming the cylindrical container 22.

As shown in FIG. 8, the feeder tube 32 is secured to the opposite, proximal, end of the container 22. The feeder tube 32, in one embodiment, is substantially rigid and may be made of a form-retaining material such as plastic, metal, or other material. The feeder tube 32 has an inner diameter that is substantially equal to the inner diameter of the container inner wall surface 22a. The feeder tube 32 may have an annular groove 40 around its outer circumferential surface at a distal end thereof, which groove 40 may serve a similar function as groove 42 discussed above. Specifically, sutures 51 may be used to secure the feeder tube 32 to the cell container 22. The sutures 51 are preferably tied around an outer circumferential surface 22b of the container 22, near the proximal end thereof, and in line longitudinally with the annular groove 40 in the feeder tube 32. The sutures 51 cause the container 22 to exert an inward force against the feeder tube 32 and to deform the container 22 locally into the space defined by the annular groove 40 to provide a secure connection. Other connecting arrangements may also be used, as can be appreciated from prior discussions.

As shown in FIG. 10, the cell barrier 24 may be provided within the container 22 during vascularization of the container 22. The cell barrier 24 serves to prevent the formation of vasculature within the inner lumen or space 23 of the container 22 (the void or space defined by the interior wall 22a of the container 22). Accordingly, the cell barrier 24 has an outer configuration or diameter that generally conforms to the inner configuration or diameter of the container 22, with little or no space therebetween. In some embodiments, the cell barrier 24 may be impervious to all materials. In other embodiments, it may be desirable to supply the container 22 with angiogenic growth factors or other agents (described in detail below) to aid in the formation of blood vessels on and surrounding the container. Accordingly, the cell barrier 24 may be formed of a material that is permeable to such agents, while remaining impervious to vasculature. In this way, such agents may be supplied from pump 52 to feed the agents into the inner lumen 23, through the cell barrier 24, and to the container 22 and surrounding region.

In one embodiment, the cell barrier 24 may be configured to have an outer configuration or diameter that is smaller than the inner configuration or diameter of the container 22 in order to permit the formation of a network of vasculature in a space of predetermined size between the cell barrier 24 and the container 22. As shown in FIG. 10B, a cell barrier 24 is shown in the top part of the Figure as being configured to generally conform to the inner surface 22a of the container 22. The bottom part of the Figure shows the cell barrier 24 having an undersized configuration such that a vascular plexus 25 may be formed in the space between the outer surface of the cell barrier 24 and the inner surface 22a of container 22. The vascular plexus 25 may have a thickness in the range of about 100 to about 200 microns and may advantageously promote increased contact area between an implanted therapeutic construct (e.g., islet cells) and the vasculature, allowing for improved transport of nutrients and other materials and agents.

In one embodiment, cell barrier 24 is formed of expanded polytetrafluoroethylene (ePTFE) or other suitable material. In addition, the cell barrier 24 may contain holes or pores of between about 1 to 2 microns in diameter to allow the passage of nutrients and other agents to the container to promote vascularization while blocking vessel formation in the inner lumen 23. In another embodiment, the holes or pores are less than 1 micron in diameter. The cell barrier 24, in one embodiment, has a wall thickness of between about 40 to 350 microns so as to minimize difficulty in passing agents and nutrients to the container 22. A relatively thin cell barrier 24 may be more conducive to material transport than a relatively thick cell barrier 24. A relatively thick cell barrier 24, however, may provide additional structural integrity so as to enable the use of a relatively thin barrier support 26. On the other hand, a thin cell barrier 24 used in conjunction with a barrier support combines advantages of both good material transport and good structural characteristics.

It should also be appreciated that, in an alternate embodiment, the functionality of the cell barrier 24 may be integrated into the structure of the container 22. For example, the container 22 may have larger pores formed in the outer cylindrical wall 22b thereof and significantly smaller pores in the inner cylindrical wall 22a thereof. In this manner, the larger outer pores would allow vessel formation therein, while the smaller pores allow passage of various agents from the lumen 23 to the vessels, but are sufficiently small to substantially prevent ingress of vessels into the lumen 23. It can thus be appreciated that in some embodiments, the previously described cell barrier 24 may be considered to be an inner portion of container 22. From this standpoint, the smaller pore inner region of the integrated structure, or the separate cell barrier structure 24 of the previous embodiment, can both be considered to be inner portions of a two portion cell container (i.e., a larger pore outer container portion and a smaller pore inner container portion).

In one embodiment, the cell barrier 24 may instead be formed of a non-porous hydrogel, such as an alginate-based hydrogel. A hydrogel cell barrier 24 may have solubility-selective material transport characteristics, whereas porous cell barriers (e.g., ePTFE) may be selective based on molecular size of the material passing through. e.g. Such solubility-selective characteristics of a hydrogel cell barrier may improve cell viability for certain applications. In some embodiments, a hydrogel cell barrier 24 may have improved material transport characteristics for oxygen, glucose, nutrients, insulin, etc., compared with porous cell barriers (e.g., ePTFE, ultrafiltration membranes, etc), as transport of species occurs in the aqueous phase which occupies the entire volume of a hydrogel membrane and thereby improves cell viability. Porous cell barrier materials, on the other hand, can be engineered for selective transport of materials. In some embodiments, for example, the porous cell barrier material has linear (“straight through”) pores, while in other embodiments they may have tortuous paths (e.g. as found in ultrafiltration membranes). A linear arrangement of pores will pass more material, at a faster rate, in comparison with tortuous pores of the same diameter. A hydrogel cell barrier 24 is permeable in that it will allow certain agents and materials to diffuse therethrough.

For embodiments utilizing the separate porous cell barrier 24, the vascularization assembly 100 may further comprise the aforementioned barrier support 26. The barrier support 26, in one embodiment, is a rigid material having a tubular configuration with an outer dimension that is slightly less than the inner dimension of the cell barrier 24, such that the cell barrier 24 fits between the barrier support 26 and container 22, as shown in FIG. 10. In one embodiment, each of the container 22, cell barrier 24 and barrier support 26 has a tubular, cylindrical configuration. The barrier support 26 is provided in order to provide form and structural support for the otherwise flaccid cell barrier 24 when inserted into the container 22. The barrier support 26 maintains the outer surface of cell barrier 24 in close or abutting relationship with the inner surface or wall 22a of the container 22 to help the cell barrier 24 carry out its primary function of preventing the development of blood vessels within the container 22.

As shown in FIG. 10, in one embodiment, both the cell barrier 24 and the barrier support 26 have a longitudinal length that is greater than a longitudinal length of the container 22, such that portions of the cell barrier 24 and barrier support 26 extend outwardly from the container 22. In the illustrated embodiment, the barrier support 26 has a perforated cylindrical wall portion 44 along at least substantially the length of the barrier support 26 that is disposed within the inner lumen 23. In the illustrated embodiment, the barrier support 26 has an imperforate cylindrical wall portion 45 along portions of the barrier support 26 that extend outwardly from the inner lumen 23. The imperforate wall portion 45 provides an impermeable conduit into the lumen or space 23 through which agents (such as growth factors) or other nutrients can be transmitted. As described later, however, portion 45 may also be perforated.

The perforated wall portion 44 of the barrier support 26 is configured to allow the passage of growth factors and/or other nutrients or agents that are supplied (e.g., that are pumped) to the lumen 23 to reach the cell barrier 24 and to then pass through the cell barrier 24 to reach the container 22. The size of the perforations of the barrier support 26 need not be limited or otherwise specifically configured, since the primary purpose of the barrier support 26 is to provide structural support for the cell barrier 24 and need not act as a permeation regulator. Further, as the distal portion of the barrier support 26 is stopped by plug 30, that portion may or may not be perforated since little or no materials are likely to flow through that region. Moreover, one of skill in the art will appreciate that the cell barrier 24 and barrier support 26 elements may be integrated into a single structure by using a material having adequate permeative and structural properties.

In the embodiment of FIG. 4, the cell barrier assembly 225 is not hollow, but comprises a trapezoidal block of permeable material. In one embodiment the block of permeable material is made from a permeable ePTFE or a sintered material, while the hollow container 22 is made from a scaffold material in accordance with the teachings of Zilla's U.S. Pat. Nos. 6,616,699 and/or 6,702,848, which are incorporated by reference.

The flat trapezoidal barrier assembly 225 is inserted into a trapezoidal flat cavity 223 within the trapezoidal container 22. The cavity is defined by interior surfaces of container 22 that correspond substantially to the outer surface configuration of the cell barrier assembly 225. The cell barrier assembly 225 is inserted into the corresponding trapezoidal cavity 223 in the trapezoidal container 22, and growth factors are pumped into the trapezoidal block cell barrier assembly 225 through a catheter 36. The catheter 36 may be received in a bore drilled or otherwise formed in the block of material forming cell barrier assembly 225. The catheter 36 may have holes in the sides thereof, in addition to the end opening thereof, to deliver growth factor to the cell barrier assembly 225. The pumped growth factor permeates through the sintered material, and through the porous container, to facilitate the formation of vessels on and within the pores formed in the container 22. The sintered material, or otherwise formed permeable material, of the cell barrier 225 is such that while it allows growth factor to pass therethrough, it is selectively permeable such that it substantially prevents ingress of vessels therein.

After the trapezoidal container 22 is sufficiently endowed with vasculature, the cell barrier 225 is removed. Subsequently, as shown in FIG. 4A, islet cells, e.g., loaded in a trapezoidal hydrogel construct 214, may be inserted into the trapezoidal cavity 223 within the container 22. The trapezoidal shape of a hydrogel construct 214 and corresponding trapezoidal shape of the cell container cavity 223 facilitates insertion of the hydrogel construct 214 into the container 22. This configuration may be particularly beneficial for use with a relatively flat trapezoidal islet-hydrogel construct for insertion into a flat trapezoidal container cavity.

FIG. 10C shows substantially all of the elements that make up the device during vascularization and shows the flexible catheter 36 forming a U-shape such that the pump 52 is positioned alongside the vascularization assembly 100. This reduces the overall length of the device. The venting tube 75 can also be positioned in a U-shaped configuration to reduce the length of the device. The venting tube 75 is illustrated as having a venting clip 77 thereon to prevent materials from egress into the container through the tube 75 during normal use of the device while implanted. The clip 77 can be removed prior to removal of the cell barrier assembly 125 and/or a gel construct 14, so that the distal end 75d of the tube 75 may be opened to allow the passage of air from or to the inner lumen 23 of the cell container 22 during insertion or removal of the barrier assembly 125 (or other components such as the gel construct 14). The clip 77 is positioned close to the feeder tube 32 opening so that they may both be manipulated at the same time during surgery and with minimal surgical openings.

In another embodiment, a syringe containing saline or other suitable fluid is connected to the distal end 75d of vent tube 75.

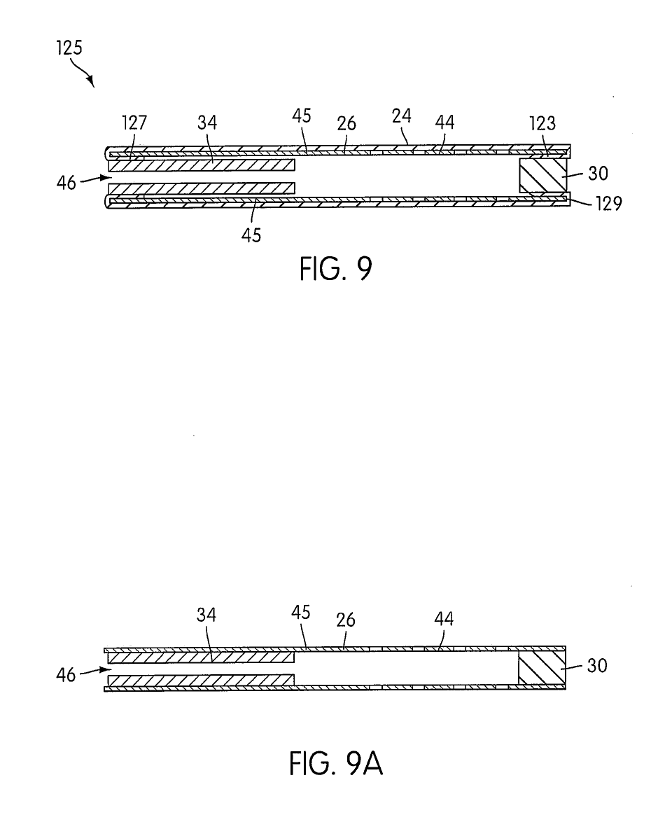

As shown in FIG. 9, prior to implantation the cell barrier 24 is disposed over the barrier support 26. Because the cell barrier 24 may be made of a relatively soft and flexible material, it is slipped over the barrier support 26 and each of its ends (distal end 123 and proximal end 127) are pushed into the interior of the barrier support 26, as shown.

As illustrated, the portion 123 of the cell barrier 24 that is folded into the interior of the perforated wall portion 44 (distal end portion) of the barrier support 26 may be held in place by insertion of a support plug 30. Specifically, the folded end portion 123 is trapped between the plug 30 and the barrier support 26. The support plug 30 may be formed of rubber, synthetic rubber, elastomer, polymer, plastic, or other suitable material. The support plug 30 resides within an end of the barrier support 26, at the perforated side 44 thereof, by a friction fit to ensure that the cell barrier 24 is held in place over the barrier support 26. One of skill in the art will appreciate that any means to retain the cell barrier 24 in place over the barrier support 26 may be used as an alternative to the support plug 30.

At the other, proximal, end of the barrier support 26, the inwardly folded end portion 127 of cell barrier 24 may be held in place by a centrally cored plug 34. As with the insertion of the support plug 30 in the distal end of the barrier support 26, the cored plug 34 may likewise be inserted into the proximal end of barrier support 26 after draping the proximal end portion 127 of cell barrier 24 around the end of the barrier support 26 and into its interior. A friction fit may be applied to sandwich the proximal end portion 127 of cell barrier 24 between the barrier support 26 and the cored plug 34 in order to hold it in place over the barrier support 26. The cored plug 34 may be formed of rubber, synthetic rubber, elastomer, polymer, plastic, or other suitable material. As shown, the cored plug 34 has a bore 46 passing axially therethrough for receipt of tubing or catheter 36.

As mentioned previously, in one embodiment, the wall portion 45 of barrier support 26 may be perforated. This is because feeder tube 32 may operate as the imperforate conduit that provides nutrients and agents. However, in another embodiment, the feeder tube 32 can be omitted, and the imperforate wall portion 45 may serve as the feeder tube.

A cell barrier assembly 125 (see FIG. 9), which includes the cell barrier 24, the barrier support 26, the support plug 30, and the cored plug 34, may then be inserted into the cell container assembly 150, which includes the cell container 22, end plug 28, and feeder tube 32 secured thereto (see FIG. 8). The cell barrier assembly 125 is inserted into the inner lumen 23 of the cell container 22, via the feeder tube 32, until its distal end 129 abuts the end plug 28. The cell barrier assembly 125 may be held in place within the container assembly 150 by securing an end cap 38 around and in a tight-fitting relationship with the feeder tube 32. The end cap 38 has a cylindrical wall portion 39 and end wall portion 41. The end cap 38 may be secured onto the feeder tube 32 by way of a friction fit between the inner surface of cylindrical wall portion 39 and outer surface of feeder tube 32, or it alternatively may be formed of a relatively softer material that can be secured onto the feeder tube 32 with sutures. One of skill in the art will appreciate that the end cap 38 may also be secured to the feeder tube 32 in any appropriate manner. The end wall portion 41 of end cap 38 provides a stop structure that prevents the cell barrier 24, barrier support 26, and plugs 30 and 34 from being withdrawn from feeder tube 32 and container 22.

The end wall portion 41 of end cap 38 may comprise a bore 48 passing therethrough. The bore 48 is in line axially with the bore 46 of the cored plug 34 in order to accommodate a pump connection line 36, such as a catheter or tubing. The catheter 36 may be passed through the bore 48 in the end cap 38 and then through the bore 46 in the cored plug 34 in order to create a supply line from the outside of the assembly to the inner lumen 23 of the cell container 22. The catheter 36 and each of the bores 46, 48 are preferably in tight-fitting relationship so as to seal the respective interfaces therebetween. The catheter 36 is connected at a proximal end thereof to a pump 52 that may be configured to supply various agents and/or materials such as vascular growth factors, immunosuppressants, nutrients, or other materials to facilitate and enhance vascularization of the cell container 22.

The catheter 36 may include a mushroom-shaped end portion 37. Upon being inserted into the cored plug 34, the catheter end portion 37 may be compressed so as to fit within the bore 46. When the end portion 37 emerges from the bore 46, it may return to its normal shape and thereby prevents the catheter from slipping out of the cored plug 34 by having a surface 37a in abutting relationship with an end surface 34a of the cored plug 34. Thus, under normal operating circumstances, the catheter 36 is prevented from slipping out of the vascularization assembly 100. When it is desired to remove the cored plug 34 from the barrier support 26, however, a force may be applied to the catheter 36, which in turn applies a force to the end surface 34a of the cored plug 34 and thereby removes the cored plug 34 from the barrier support 26.

In an alternative embodiment, the catheter 36 is inserted into the cored plug 34 prior to attachment of the end cap 38 to the feeder tube 32. In such an arrangement, the catheter 36 is inserted into the bore 48 in the end cap 38 before being inserted into the cored plug 34. The catheter 36 is then inserted into the cored plug 34 and the end cap 38 is slid along the catheter 36 until it comes into engagement with and is secured onto the feeder tube 32 in a manner discussed above. In embodiments in which the catheter 36 includes a mushroom-shaped end portion 37 as described above, the catheter 36 may be pushed through the bore 46, as described, or the cored plug 34 may instead be disposed over the catheter 36 from an opposite end, obviating the need to compress the end portion 37 in the bore 46.

The vascularization assembly 100, which comprises the cell container assembly 150, the cell barrier assembly 125, the pump 52, and the inserted catheter 36, is then ready for implantation into a living body for vascularization. The vascularization assembly 100 is preferably implanted subcutaneously by known methods. Other sites may alternatively be utilized for implantation that offer a more convenient or effective process. By natural operation of various cells within an organism, new blood vessels will tend to form around the cell container 22 and into its interconnecting pores.

In an embodiment, an applicator may be used to implant the cell therapy device 10. For the cell therapy device 10 that includes the container 22 having a tubular shape, the application may be made from, for example, a polyethylene tube that has a slit along a length thereof. For embodiments in which the container 22 has a substantially trapezoidal shape, the applicator may have a channel shape and may be made from an oriented polyethylene terephthalate (PET) sheet (e.g. Mylar® made by E. I. DuPont de Nemours). The applicator for the trapezoidal container 22 may also include an inner sheet of polytetrafluoroethylene (PTFE), such as Teflon® made by E. I. DuPont de Nemours, which may smoothen the surfaces of the applicator. The cell therapy device 10 may be inserted into the applicator, which then may be inserted into the implant site. Once the cell therapy device 10 has been delivered to the implant site, the applicator may be removed, while leaving the cell therapy device 10 at the implant site. For the channel-shaped applicator, the inner sheet of PTFE may be removed once the applicator is positioned at the implant site prior to delivery of the cell therapy device. Use of an applicator may decrease implantation time and/or reduce trauma to the patient during implantation.

In an alternative embodiment, shown in FIGS. 8A, 9A, and 10A, the cell barrier 24 is attached to the cell container assembly 150 prior to vascularization and insertion of the barrier support 26. The cell barrier 24 may be left within the cell container 22 after vascularization and remains there during the insertion of therapeutic materials, such as islet cells. In this arrangement, the cell barrier 24, instead of being secured at each end of the barrier support 26, may be secured at its distal end between the end plug 28 and the cell container 22 and at its proximal end by the feeder tube 32, as shown in FIG. 8A, or by another fastening element (such as a large center bored plug), or it may be kept free. Accordingly, the barrier support 26 may still be used to maintain the form of the cell barrier 24 and to keep it against the inner wall 22a of the cell container 22, and is inserted into the container 22 without the cell barrier 24 attached, as shown in FIGS. 9A and 10A. After vascularization, the barrier support 26 will be removed while the cell barrier 24 remains in place within the cell container 22. In other embodiments, the barrier support 26 may also be kept within the cell container 22.

B. Vascularization of the Implanted Container

While blood vessels may form naturally in and around the cell container 22 of the implanted vascularization assembly 100, the process may be stimulated, accelerated, and/or induced by the delivery of various materials into the tissues surrounding the assembly 100. Accordingly, methods and compositions for promoting vascularization of an implanted device 10 are discussed below. The term “vascularization” as used herein in relation to the implanted device includes, but is not limited to, inducing formation of blood vessels in the tissue at the implant site The terms “angiogenesis” or “neovascularization” may be used interchangeably with “vascularization” in the present disclosure. The term “pre-vascularization” may also be used according to the understanding that the cell container 22 is generally vascularized prior to living cells being introduced thereto. It is understood that, during blood vessel formation, at least some of the blood vessels are in direct contact with the porous outer surface 22b, and at least some blood vessels will be formed (or “grow”) within the pores of container 22.

U.S. Pat. No. 6,638,520 B1 to Sanders, hereby incorporated by reference in its entirety, discloses contacting a biocompatible material with an angiogenic biological material, allowing blood vessels to grow from the angiogenic biological materials into the biocompatible material, separating the angiogenic biological material from the biocompatible material, and incorporating the biocompatible material into an implantable medical device to promote surface vascularization of the implanted device and reduce or eliminate the foreign body reaction. Angiogenic biological materials disclosed in that patent include, e.g., chorioallantoic membranes of birds' eggs, aortic tissue, and corneal tissue. It is contemplated that some of these teachings may be used in the context of the present invention.

U.S. Pat. No. 7,029,838 B2 to Williams et al., hereby incorporated by reference in its entirety, discloses methods and materials to form “prevascularized constructs” wherein harvested microvessel fragments are placed in a three-dimensional culture matrix under conditions to induce formation of capillary beds in the construct, to form prevascularized construct designed to connect with the vasculature of a host animal following implantation. The prevascularized construct may be used to vascularize engineered tissue prior to implantation into a subject, or may be used to vascularize engineered tissue after the engineered tissue is implanted. It is contemplated that some of these teachings may be used in the context of the present invention.

Exogenous angiogenic factors have been used to induce vascularization of an implanted device or implant site. U.S. Patent Application Publication No. US 2005/0180957 A1 to Scharp et al., hereby incorporated by reference in its entirety, discloses implanting cells, tissues or organs, which may be encapsulated or unencapsulated, combined with fibrin-bound angiogenic growth factors to enhance promote vascularization at the implant site and enhance survival and function of the implanted cells, tissues, or organs. It is contemplated that some of these teachings may be used in the context of the present invention. Prokop et al. disclose attempts to vascularize allogenic islet cell implants using coating biocompatible meshes coated with hydrogels having incorporated acidic or basic fibroblast growth factor (a-FGF or b-FGF) in an electrostatic complex with polyelectrolytes and/or with heparin to provide release of angiogenic growth factor (Prokop et al., 2001, Diabetes Tech. Ther. 3:245-261, hereby incorporated by reference in its entirety). It is contemplated that some of these teachings may be used in the context of the present invention.

Vascularization of the implanted device 10, in embodiments in which islet cells are subsequently loaded to treat Type I diabetes, provides surfaces across which insulin moves out of the device and into blood vessels, and connections to routes for circulation of insulin throughout the body. Separately, vascularization of the implanted device may support and sustain the functioning of the device by providing nutrients, carrying out gas exchange, removing cellular wastes, and inhibiting fibrous encapsulation.

In accordance with one aspect of the invention, a method for vascularizing the implanted device 100 is provided. The method includes implanting the device 100 and, prior to the device being loaded with islet cells or other therapeutic agent, delivering a growth factor composition that includes autologous platelet gel serum (APGS) to the device for a sufficient period of time that formation of new blood vessels is induced in the tissues surrounding the implant site. At least some of the new blood vessels are in direct contact with the porous outer surface and/or formed in and around the pores of the cell container 22 of the device 10. As discussed above, in one embodiment, the pores of the container wall are large enough to permit growth of small blood vessels into the pores until they reach the cell barrier 24. Growth of small blood vessels or capillaries (or microcapillaries) into the pores of the porous outer surface 22 may enhance exchange of molecules provided in the interior cavity or inner lumen 23 of the device and the tissues surrounding the devices. Such blood vessels or capillaries may, in a non-limiting example, be dimensioned on the order of about 5 to 20 microns lengthwise.

After a sufficient level of vascularization is achieved, therapeutic biological materials, such as islet cells, are introduced to the inner lumen 23 of the device 100, with the therapeutic biological materials being separated from the blood vessels by at least the permeable cell barrier 24.

In accordance with another aspect of the invention, therapeutic cells can include cells with a high angiogenic potential including, but not limited to, adipose derived stromal cells, mononuclear cells from the blood, bone marrow derived cells, genetically modified cells, or other cell types having high angiogenic potential known to those of skill in the art. The cells with a high angiogenic potential may be autologous cells isolated from the patient into which they are to be implanted. The autologous cells with a high angiogenic potential would be introduced into the vascularization device at the time of implantation and remain in the device for a period sufficient to achieve a desired result, e.g., a sufficient level of vascularization of the implanted device 100. The cells with a high angiogenic potential may be removed prior to introduction of other therapeutic cells, e.g., islet cells, or may remain in the device after other therapeutic cells are introduced.

The level of vascularization of the implanted device 100 can be determined by one of skill in the art using any of a variety of accepted methods. The methods and compositions provided herein should provide vascularization sufficient to support the function of the implanted device 100, e.g., to supply nutrients and oxygen to implanted islet cells in the inner lumen 23 of the device 100, and provide sufficient uptake of insulin produced by the implanted islet cells.

A non-limiting description of various approaches is provided below. In one exemplary embodiment, one of skill in the art can adapt methods for measuring tumor vascularization to measure vascularization of the implanted device 100. Suitable methods include, but are not limited to, laser Doppler perfusion imaging (Couffinhal et al., 1998, Am. J. Pathol. 152: 1667-1679), hereby incorporated by reference, microdoppler ultrasound imaging (Gambetti et al., 1993, Ultrasound Med. Biol. 19:763-76), hereby incorporated by reference, X-ray microangiography (Gole et al., 1990, Doc. Opthalmol. 74: 163-169; Gundersen et al., 1978, J. Microsc. 113: 27-43), hereby incorporated by reference, fibered confocal fluorescence microscopy (FCFM) combining confocal fluorescence microscopy with fiber optics (Laemmel et al., 2004, J. Vasc. Res. 41:400-11; Ritman E. L., 2004, Annu. Rev. Biomed. Eng. 6:185-208), hereby incorporated by reference. It is understood that one of skill in the art may select and adapt any suitable method in light of advantages and limitations of the method for determining vascularization of implanted device 100. In another exemplary embodiment, the oxygen status of the tissues adjacent to the device 100 can be measured as a surrogate indicator of vascularization.

In another exemplary embodiment, vascularization can be measured using histological approaches, e.g., using stains, antibodies, and other molecules that can be used to identify cell types. In an exemplary embodiment, antibodies that bind cell surface markers can be used, e.g., anti-factor VIII and anti-collagen Type IV antibodies, anti-CD31 antibodies, and GSL I-B4 lectin (See, Prokop et al., 2001, Diabetes Tech. Ther. 3:245-261, hereby incorporated by reference). In an exemplary embodiment, vascularization can be measured by preparing fixed tissue samples and measuring cell density, e.g., using DAPI staining, as well as cell type using stains, antibodies, and other markers to identify distinct cell types, e.g., antibodies against endothelial cell markers such as CD31, CD34, or von Willebrand factor, in the fixed samples. In another exemplary embodiment, the fixed sample may include at least a portion of an implanted device, in order to measure tissue ingrowth and angiogenesis, i.e., vascularization, of the implanted device.

In yet another exemplary embodiment, one of skill in the art can carry out functional measurements of vascularization, e.g., by measuring nutrient diffusion, gas exchange, and/or insulin uptake in the tissues at the implant device 100, lectin perfusion, or by micro-CT imaging. In yet another exemplary embodiment, one of skill in the art may develop a model for predicting when a sufficient level of vascularization has taken place, using information such as biometric measurements (patient size, age, sex, etc.), composition and delivery rate of the growth factor composition (in particular, delivery of angiogenic factors) during vascularization, the amount of therapeutic biological materials (e.g., islet cells) to be implanted, and the results of previous implantation trials.

C. Growth Factor Compositions

In accordance with another aspect of the invention, growth factor compositions for promoting vascularization of the implanted device are provided. The growth factor compositions may include growth factors from exogenous sources, e.g. exogenous VEGF, and/or growth factors from autologous sources, e.g., autologous platelet gel serum (APGS) derived from the patient or subject receiving the implanted device. The growth factors may also include additional autologous components and/or exogenous (non-autologous) components. Administering an autologous composition should provide growth factors and other components that are expected to be biologically active in the patient from which the APGS was obtained, and should avoid potential negative effects of exogenous compositions, e.g., problems with purity, stability, effectiveness, or immunogenicity of various components. It is understood that any exogenous (non-autologous) components included in the growth factor composition should be suitable for administration to the patient receiving the implanted device.

Autologous platelet gel serum (APGS) is a defibrinated, substantially cell-free preparation that contains the released contents of platelets and other soluble contents of platelet rich plasma (PRP), and may contain factors released from white blood cells (WBC). Briefly, whole blood is obtained from the patient, and PRP is prepared by separating platelets from other blood components (e.g., red blood cells) to obtain a fraction highly enriched in platelets. Various methods for preparing PRP can be used. A non-limiting exemplary embodiment is disclosed in US 2006/0136050 (“the '050 Publication”), hereby incorporated by reference in its entirety, wherein PRP is generated by variable speed centrifugation of whole blood using the Magellan™ Autologous Platelet Separator System (Medtronic, Inc., Minneapolis, Minn.). The '050 Publication describes the device as being well-suited for producing PRP from a small amount of autologous blood in a closed system that minimizes contamination. In the next step, PRP is clotted to form autologous platelet gel (APG). In a non-limiting exemplary embodiment, APG is formed by adding thrombin or a thrombin alternative to PRP and allowing the mixture to stand at room temperature for about 15 to 30 minutes until a viscous gel, i.e., APG, is formed. APG (clotted PRP) is then centrifuged, and the supernatant is recovered as APGS. It is understood that one of skill in the art can prepare PRP, APG, and APGS using other methods and devices known in the art.

Platelet contents provide many of the growth factors found in APGS. Platelets are derived from marrow megakaryocytes, have no nucleus for replication, and have an expected lifetime of five to nine days. Platelets contain pre-formed growth factors that are released upon activation (and rupture). The PRP from which APGS is obtained, may also contain white blood cells (WBCs) which, following activation, secrete a variety of factors, including but not limited to, growth factors, cytokines, chemokines, prostaglandins, and matrix metalloproteinases.

The chemical composition of APGS can include, but is not limited to, one or more of the following: platelet-derived growth factor (PDGF, PDGF-BB), platelet-derived epidermal growth factor (PDEGF), heparin-binding epidermal growth factor (HB-EGF), fibroblast growth factor (FGF, including acidic FGF (a-FGF) and basic FGF (b-FGF)), transforming growth factor-β (TGF-β) transforming growth factor-α (TGF-α), insulin-like growth factor (IGF), platelet-derived angiogenesis growth factor (PDAF), placental growth factor (PLGF or P1GF), angiopoietin 1 (ANG1), angiopoietin 2 (ANG2), vascular endothelial growth factor (VEGF, include VEGF-A, VEGF-165, VEGF-C), hepatocyte growth factor (HGF), keratinocyte growth factor (KGF), brain-derived neurotrophic factor (BDNF), tumor necrosis factor α(TNFα), thrombopoietin (TPO), tissue inhibitor of metalloproteinase 1 and 2 (TIMP 1, TIMP 2), cytokines such as granulocyte colony stimulating factor (G-CSF), granulocyte macrophage colony stimulating factor (GM-CSF), chemokines, prostaglandins, matrix metalloproteinases and/or other components.

In one exemplary embodiment, whole blood from human donors and processed to obtain normal serum and APGS. APGS was analyzed using enzyme-linked immunosorbent assay (ELISA) to determine the levels of VEGF, bFGF, PDGF-BB, PLGF, BDNF, TNFα, TGFα and β, GMCSF, HB-EGF, HGF, KGF, Ang2, TPO, TIMP 1 & 2. Normal serum was also analyzed and the levels of VEGF, bFGF, PDGF-BB, PLGF, BDNF, TNFα, TGFα and β, GMCSF, HB-EGF, HGF, KGF, Ang2, TPO, TIMP 1 & 2 were compared with those in APGS.

In accordance with one aspect of the invention, at least a portion of the angiogenic factors in APGS remain stable under conditions found in the implanted device. In one exemplary embodiment, samples of APGS and normal serum were incubated at 37° C. in an Alzet® osmotic pump for 14 days. Levels of angiogenic factors were measured prior to incubation and after 14 days of incubation. Ang2 declined sharply in both normal serum and APGS after 14 days of incubation. The rest of the angiogenic factors tested, e.g., VEGF, did not decline substantially in normal serum or APGS after 14 days of incubation at 37° C.

In one exemplary embodiment, a comparative analysis of angiogenic factors in APGS from women who are Type I diabetics (n=3) or non-diabetics (“normal” n=3) was carried out using ELISA to measure levels of the angiogenesis-related proteins VEGF, b-FGF, PLGF, ANG2, BDNF, TGF-β, and PDGF-BB in APGS preparations from each subject. The results showed that Type I diabetics appeared to have higher average levels of all the angiogenic factors tested. In particular, the Type I diabetics had an average concentration of b-FGF that was over 5 times the average concentration in non-diabetics. Type I diabetics also appear to have a significantly higher concentration of VEGF and ANG2 than non-diabetics.

The chemical composition and biological effects of the growth factor composition containing APGS can be modified by one of skill in the art. The concentration of various components of the composition, in particular angiogenic growth factors, can be changed by adding additional components, removing certain components, or altering the effective concentration of certain components. In one embodiment, an APGS preparation from a patient who is to receive an implanted device is analyzed for angiogenic factors and, as a result of the analysis, it may be determined that the levels of one or more angiogenic factors in the APGS preparation should be altered before the growth factor composition containing APGS is administered to the patient.

In accordance with one aspect of the invention, the level(s) of at least one angiogenic factor present in an APGS preparation can be altered. In one exemplary embodiment, the level of an angiogenic factor present in an APGS preparation indicates that the level in the growth factor composition to be administered to the patient should be increased and the level is increased by adding an additional amount of the angiogenic factor. Examples of factors that may be added include, but are not limited to, VEGF, PDGF-BB, stromal cell-derived factor (SDF-1), Ang1, Ang2, b-FGF, and other factors known to one of skill in the art. In one embodiment, the angiogenic factor is autologous in origin. In another embodiment, the level of the angiogenic factor is increased by fractionation or other manipulation of the APGS preparation to obtain APGS enriched in the factor. In another embodiment, the effective level of the factor is increased by adding components that increase the biological effectiveness of the angiogenic factor. In another embodiment, the effective level of the angiogenic factor is increased by adding components that decrease the effectiveness of antiangiogenic factors, e.g., antibodies against antiangiogenic proteins such as endostatin, angiostatin, interferons, platelet factor 4, prolacting 16 kD fragment, or thrombospondin.

In another exemplary embodiment, the level of an angiogenic factor present in an APGS preparation indicates that the level in the growth factor composition to be administered to the patient should be decreased. In one embodiment, the APGS preparation is contacted with an antibody that specifically binds the angiogenic factor. The total amount of the component in the APGS preparation can be reduced by separating the bound antibody-bound-component complex from the rest of the APGS preparation, e.g., using antibody-binding media. In one embodiment, the antibody is autologous in origin. In one exemplary embodiment, anti-TIMP1 is added to an APGS sample, and the measured level of TIMP1 in the APGS sample is reduced. In another embodiment, the level of the angiogenic factor is decreased by fractionation or other manipulation of the APGS preparation to obtain APGS depleted in the factor. In another embodiment, the effective level of the factor is decreased by adding components that inhibit, interfere, or otherwise decrease the biological effectiveness of the angiogenic factor. Examples of components for decreasing the actual or effective level of an angiogenic factor include, but are not limited to, protease inhibitors such as aprotinin, antibodies against angiogenic factors such as anti-TIMP1, anti-TIMP2, anti-TIMP3, or other components known to one of skill in the art.

D. Use of Growth Factor Composition to Promote Angiogenesis to Vascularize the Container

The size and types of blood vessels that form direct contact with the cell container 22 may vary, depending on various factors including, but not limited to, the structure and dimensions of the cell container 22 and pores thereof presented to blood vessels, and the ability of the surrounding tissues to support angiogenesis. It expected that capillaries or “microcapillaries” generally consisting of a single layer of endothelial cells and their basement membrane, will grow into at least the outer pores of the cell container 22. Larger blood vessels having more layers (e.g., inner endothelial layer surrounded by muscle and/or collagen outer layers) may form where structural parameters such as surface dimensions or pore size permit.

It is known in the art that angiogenesis in tissues surrounding a device can be induced, resulting in formation of new capillaries in the interstitial space and surface endothelialization. Previous attempts to promote vascularization of medical devices have used well-known angiogenic factors such as vascular endothelial growth factor (VEGF), fibroblast growth factors 1 and 2 (FGF-1, FGF-2), and others factors. Although VEGF is endothelial cell-specific, it has relatively weak mitogenic activity with respect to stimulating endothelial proliferation. FGF-1 and FGF-2 are more potent mitogens, but are less cell-type-specific. Co-owned U.S. Patent Application Publication Nos. US 2006/0095121 and US 2006/0136050, which are hereby incorporated by reference in their entirety, disclose the use of APG to coat stent grafts prior to implanting the graft, to induce endothelialization of the graft, especially the lumen surface of the graft. Similar principles can be used with respect to vascularization of container 22.

In accordance with one aspect of the invention as disclosed above, exogenous VEGF can be delivered from an implanted delivery device and promote angiogenesis in the tissue surrounding the implanted device, thus promoting vascularization of the device. In one exemplary embodiment, vessel density into container 22 increased significantly after 10 days of continuous delivery of VEGF (VEGF solution at 250 μg/ml, pumped at a rate of 1 μg/day), and vascular area increased even more significantly.

In accordance with one aspect of the invention as disclosed above, APGS contains a variety of angiogenic factors that are expected to be effective for angiogenesis in the patient from which the APGS was obtained. In an exemplary embodiment, the effectiveness of APGS to stimulate angiogenesis was compared with the effectiveness of VEGF alone, by characterizing the effect of each treatment on proliferation of endothelial cells in vitro. Endothelial cell proliferation was significantly greater in APGS-treated cells than normal serum treated cells, whereas APGS and VEGF treatments were very similar in their ability to stimulate cell proliferation.

In accordance with one aspect of the invention, growth factor composition including APGS can be delivered to the tissues surrounding an implanted device to induce or enhance angiogenesis in the tissues surrounding the device, thereby promoting vascularization of the device, In one exemplary embodiment, the effectiveness of APGS was compared with the effectiveness of commercially available VEGF, normal serum, or buffer to promote angiogenesis in vivo throughout a porous implanted container of the type disclosed herein. Briefly, a porous polyurethane container of the type disclosed herein was implanted in rats, and various treatments (APGS, VEGF, normal serum, or buffer) were pumped into the container and thereby delivered to the tissues surrounding the implanted container. After 10 days, the container was removed for histological evaluation of the container and surrounding tissues, which included endothelial-cell-specific staining with anti-CD31 antibody and digital analysis of the stained tissues to provide a quantitative measurement of the total area occupied by blood vessels. The total area occupied by blood vessels was greater after treatment with APGS or VEGF, than after treatment with normal serum or buffer. Many of the vessels detected in the stained tissues were classified as feeder vessels, which branched off into smaller vessels, including capillaries. Histological analysis of the container and surrounding tissues demonstrated that exposure to APGS or VEGF induced the formation of a more highly vascularized network throughout the tissues than did exposure to normal serum or buffer. These results demonstrate that delivery of APGS to tissues surrounding an implanted device can enhance vascularization of the implanted device as provided herein.

Further, as disclosed above, the chemical composition of the growth factor composition including APGS can be modified to increase or decrease the levels and/or biological effectiveness of various factors. It is understood that one of skill in the art can modify the disclosed methods and compositions as necessary, without departing from the methods and compositions presented in the present disclosure.

In accordance with one aspect of the invention, delivery of the growth factor composition from the implanted device to the surrounding tissues will form a gradient of angiogenic factors, where levels of angiogenic factors will be higher in tissues closer to the device, and levels will decrease with increasing distance from the device lumen or cavity. It is understood that many blood vessels will grow along the gradient toward the center device, from regions of lower angiogenic factor levels to regions of higher angiogenic factor levels.

E. Delivery of Growth Factor Composition

As discussed above, the growth factor composition can be delivered to the implanted device (vascularization assembly) 100, and emitted to the body, in particular to tissue surrounding the implanted device, as necessary to achieve the desired level of vascularization of the cell container 22.

In one embodiment, the growth factor composition including APGS is delivered continuously, or nearly continuously, to the implant, providing a constant supply of angiogenic factors to stimulate angiogenesis in the tissues surrounding the device. Non-limiting exemplary embodiments include constant, or nearly constant, delivery of APGS for about 1 day, about 2 days, about 3 days, about 4 days, about 5 days, about 6 days, about 7 days, about 8 days, about 9 days, about 10 days, about 11 days, about 12 days, about 13 days, or about 14 days or longer. In one embodiment, APGS may be delivered at a rate of about 6 microliters per day for about 9 days to about 11 days. In another embodiment, the growth factor composition including APGS is delivered in a pulsed fashion, wherein minutes, hours, or days may elapse between delivery episodes. As discussed above, delivery of the growth factor composition from the implanted device 100 to the surrounding tissues is expected to create a gradient of angiogenic factors in the surrounding tissues, such that many blood vessels will grow along the gradient towards the center of implanted device 100, from where the growth factors emanate.

In another embodiment, a growth factor composition containing VEGF as the growth factor is delivered continuously, or nearly continuously, to the implant, providing a constant supply of angiogenic factors to stimulate angiogenesis in the tissues surrounding the device. Non-limiting exemplary embodiments include constant, or nearly constant, delivery of VEGF for about 1 day, about 2 days, about 3 days, about 4 days, about 5 days, about 6 days, about 7 days, about 8 days, about 9 days, about 10 days, about 11 days, about 12 days, about 13 days, about 14 days, about 20 days, about 30 days, about 40 days, about 60 days, about 100 days, or longer.

The growth factor composition may be delivered by being coated on, or formed as a layer on, one or more surfaces of the cell therapy device 10 such that, after implantation, at least one growth factor is eluted and emitted into tissues surrounding implanted device 100 sufficient to achieve vascularization of container 22. Alternatively, the cell growth factor may be formed as a composition with a polymer and the resulting polymer system can be contained in the device, e.g., may be placed in the device to be in contact with the eluting stream, where the polymer system may be a polymer plug, cartridge, capsule, hydrogel, or other polymer matrix than can contain growth factor composition and from which at least one growth factor can be eluted and emitted to the body. Polymer systems that contain and release growth factor compositions are well known. Suitable polymer systems include, but are not limited to, biodegradable or bioabsorbable polymer systems that provide controlled release of growth factors e.g., as disclosed by Sheridan et al. (J Control Release, 64 (1-3):91-102, 2000), biodegradable hydrogels, e.g., as disclosed, by Tabata et al. (Pure Appl Chem, 70:1277-1282, 1998), microtextured surfaces, e.g., microtextured silicone and poly-L-lactic acid (PLA) surfaces as disclosed by Parker et al. (Tissue Eng. 8: 853-861, 2002). Elution (release) of growth factors from coated surfaces and polymer systems as described herein may occur by diffusion processes, driven by concentration gradients in the surrounding medium. Without wishing to be limited by this theory, elution of growth factors from coated surfaces or polymer systems may be accelerated by pumping fluids through the device, as the moving fluids passing over/through coated surfaces or polymer systems will carry away eluted growth factors and thereby maintain a concentration gradient sufficient to drive further elution. Without wishing to be limited by this theory, a growth factor composition in layer or coating form, or contained in a polymer system as described herein, may provide a means for stabilizing components of the growth factor composition. In an exemplary embodiment, 2.5 μg VEGF was loaded on the surface of heparin-coated polyurethane discs, and about 39% of the initial amount of VEGF was eluted in a first washing step at 24 hours after loading, and an additional about 10% was eluted by additional washing steps from 24 to 48 hours after loading. Growth factor composition can be coated on external surfaces of container 22, which may include pores of cell barrier 24, such that cells and/or tissues having angiogenic potential may contact the container surface and growth factor receptors on the cells may bind growth factors directly from the container surface.

The growth factor composition may be delivered as a result of being introduced into at least one lumen of cell therapy device 10, wherein the at least one lumen is positioned to be in communication with lumen 23 of container 22, such that fluid containing at least one component of the growth factor composition will be delivered to container 22 and emitted to the body, in particular to tissue surrounding the container. The growth factor composition may be introduced in fluid form into at least one lumen of device 10, including the pump 52, catheter 36, or container 22. The growth factor composition may be in the form of a layer or coating on a surface, and can be introduced into at least one lumen positioned to be in communication with lumen 23 of container 22, by elution from the coated surface into fluid in the at least one lumen. In one exemplary embodiment, lumen 23 of container 22 is loaded with growth factor composition prior to implantation of the device. In another exemplary embodiment, a separate polymer system containing growth factor composition is introduced into a lumen that communicates with lumen 23, either directly or through intervening lumens. In another exemplary embodiment, growth factor composition is coated or layered on the lumenal surface of a structure, e.g., pump 52, catheter 36, or container 22. In another exemplary embodiment, growth factor is coated or layered on surfaces of container 22 that are external to cell barrier 24. In another exemplary embodiment, a separate polymer system containing growth factor composition is introduced into a structure, e.g., pump 52, catheter 36, or container 22. In embodiments including but not limited the exemplary embodiments above, the introduced growth factor composition is delivered to the implanted device 10 or 100, and subsequently to tissue surrounding the implanted device, as necessary to achieve the desired level of vascularization of cell container 22.

The growth factor composition to be delivered may be introduced by means of a polymer system such as a porous cartridge loaded with the growth factor composition, wherein the porous cartridge can be introduced into any lumen in communication with lumen 23 of container 22, and growth factors are eluted from the cartridge into the fluid being pumped through the device. In one embodiment, the porous polymer cartridge has tortuous surfaces coated with the growth factor composition. In another embodiment, the porous polymer cartridge contains materials coated with or impregnated with the growth factor composition, e.g, microbeads, porous polyurethane, a hydrogel, or a “sponge” material such as polyvinylalcohol (PVA) sponge, such that growth factors are eluted from the materials and pass out of the pores of the cartridge. In one exemplary embodiment, the porous cartridge loaded with growth factor composition is inserted into (the lumen of) the cell container 22 or the catheter 36 as shown in FIG. 1. In another exemplary embodiment, the porous cartridge loaded with growth factor composition is inserted into (the lumen of) any of the structures of the components of the device (vascularization assembly) 100 as shown, e.g., in FIG. 6, prior to implantation. In another exemplary embodiment, a means is provided for inserting a porous polymer cartridge loaded with the growth factor composition following implantation of the device, e.g., via an external port such that the cartridge will be placed into the fluid being pumped through the device.

Different routes, means, and mechanisms for delivery of growth factor composition, including but not limited to coating surfaces and introducing growth factor composition into a lumen, may be used in combination. It is further understood that the routes, means, and mechanisms for delivery as described herein can be used to deliver other therapeutic factors implanted device, to the therapeutic cells 12, or to tissues surrounding the implanted device.

The growth factor composition may be supplied to the cell container 22 until sufficient vascularization is detected. Specimen trials may be undergone to determine desirable quantities and duration of supply of growth factors, or experiments may be carried out on each patient to determine the most suitable levels and duration of supply of growth factors. After implantation, the degree of vascularization may be determined by non-invasive means such as laser Doppler perfusion imaging, microdoppler ultrasound imaging, X-ray microangiography. fibered confocal fluorescence microscopy (FCFM), or other suitable methods known in the art.

In another embodiment, an implantable delivery device is provided that supplies at least one agent to surrounding tissues after implantation, wherein the device has a surface coated with the agent. The delivery device may have a surface coated with more than one agent, and may deliver more than one agent. The delivery device may provide various nutrients, immunosuppressive agents, growth factors, or other cell-life-promoting agents. In particular, the delivery device may be coated with growth factors, e.g., VEGF, PDGF-BB, or APGS. The delivery device may also be coated with an agent that enhances the effectiveness of another agent, or an agent that affects the environment in which the delivery device and agents are found, e.g., an anticoagulant such as heparin. The delivery device may be porous and may become vascularized after implantation. In an exemplary embodiment, porous polyurethane discs were coated with heparin and various amounts of VEGF, PDGF-BB and combinations thereof, implanted subcutaneously in rats for 10 days, and then explanted and evaluated for angiogenesis (vascularization). All treatments, including the uncoated discs, showed vessel ingrowth with similar vessel diameters. Discs coated with heparin only showed significantly greater vessel density and vascular area (% ingrowth area vascularized) than uncoated discs. Heparin-coated discs coated with 3.6 μg PDGF-BB, 12 μg VEGF, or a combination of 1.8 μgPDGF-BB and 12 μgVEGF, showed significantly greater vessel density than discs coated with heparin only. Heparin-coated discs coated with 12 μg VEGF, or a combination of 1.8 μgPDGF-BB and 12 μgVEGF, showed significantly greater vessel vascular area than discs coated with heparin only. Similar patterns were seen when discs were implanted for 2 months, except that vessel diameter decreased in heparin-coated discs coated with a combination of 1.8 μgPDGF-BB and 12 μgVEGF, and vessel density decreased in uncoated discs, discs coated with heparin only, and heparin-coated discs coated with a combination of 1.8 μgPDGF-BB and 12 μgVEGF. Only heparin-coated discs with 1.8 μgPDGF-BB showed an increase in vessel density after 2 months. Vascular area decreased significantly in all treatments after 2 months implantation.

F. Transplantation of Therapeutic Cells

Once it is determined that the cell container 22 is sufficiently vascularized, e.g., to a desired degree of vascularization as determined by any one or more of the above or other methods, therapeutic cells, e.g., islet cells or other cells, or therapeutic materials, are loaded or implanted into the inner lumen 23 of the cell container 22, where they carry out their normal functions and/or therapy in ready communication with the developed blood vessels in and around the container 22.

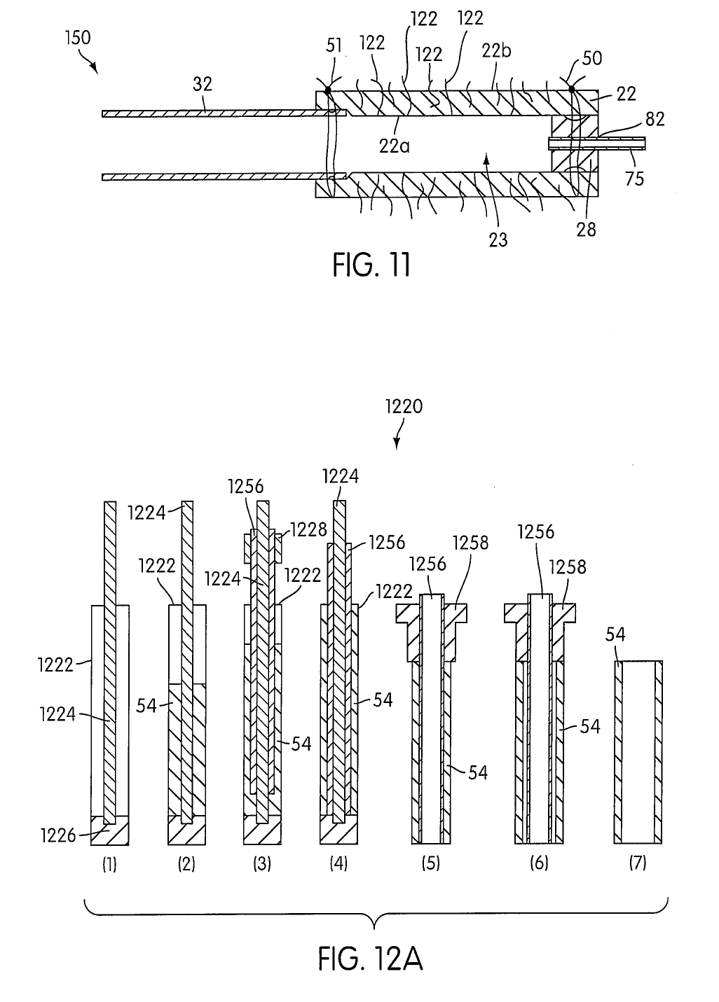

When vascularization has reached a desired level, the cell barrier assembly 125 may be removed from the container assembly 150. FIG. 11 shows the container assembly 150 with newly formed vasculature 122 after removal of the cell barrier assembly 125. Minimally invasive surgery is undergone to remove the end cap 38, pump 52, and catheter 36, and to then remove the cell barrier assembly 125. These elements are removed while keeping the container assembly 150 in place so as to prevent or minimize the disruption of the newly formed vasculature 122 in and around the container 22. The cell barrier assembly 125 may be removed via the feeder tube 32 such that the feeder tube is held in place with tweezers, for example, while the cell barrier assembly 125 is gripped with other tweezers and pulled out. In this manner, the cell container 22 need not be held or otherwise handled, thereby reducing any possible impact to the newly formed vasculature.

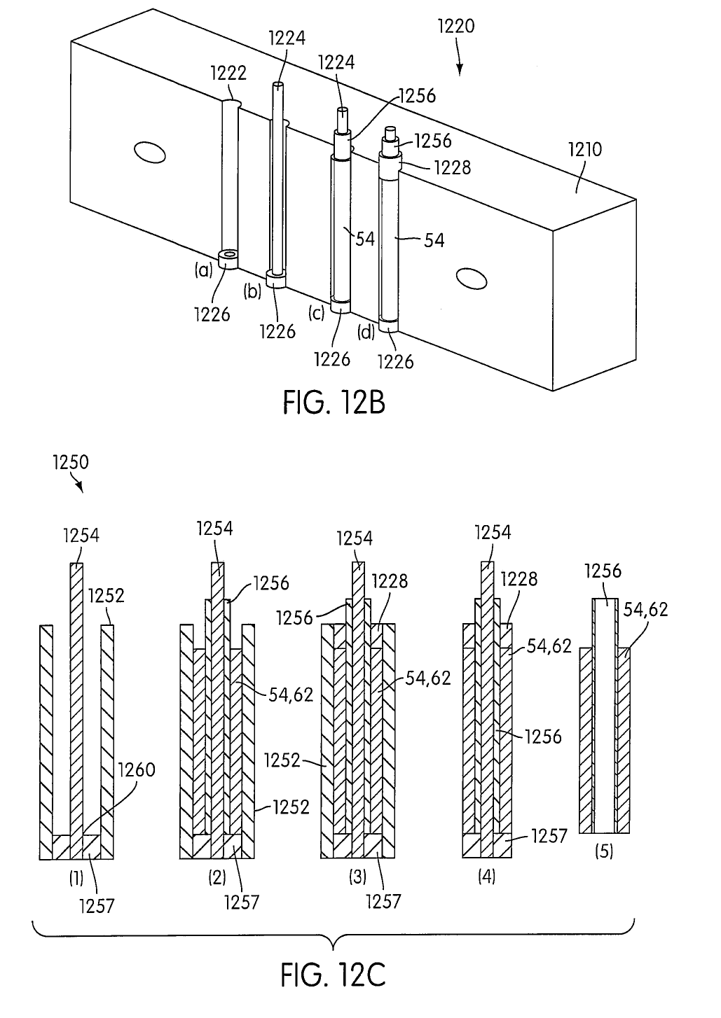

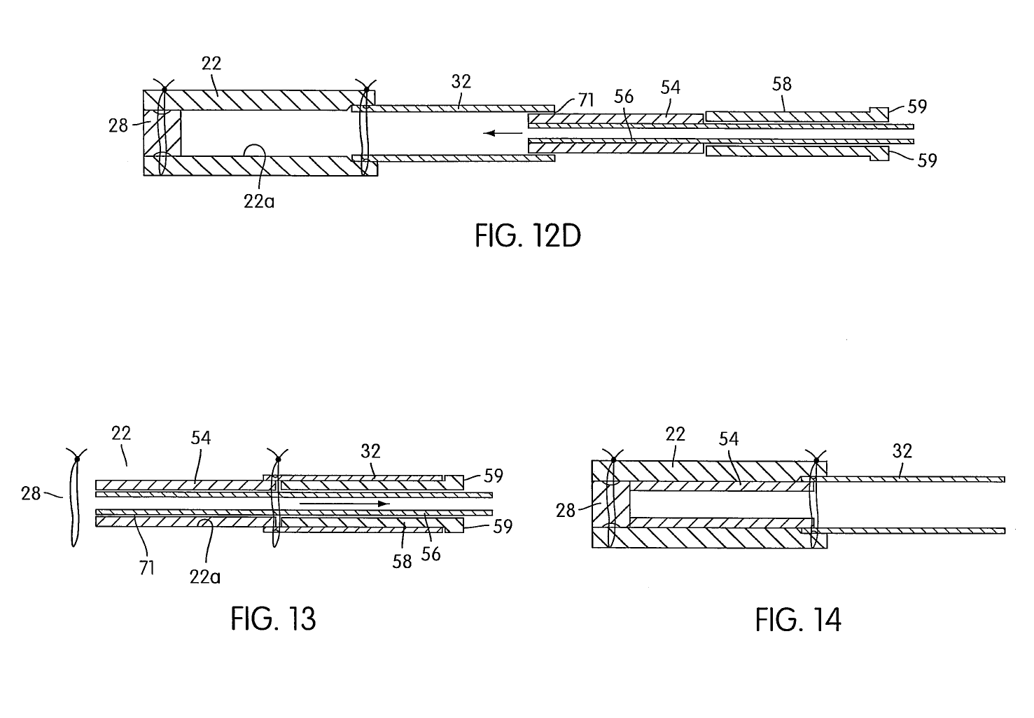

Therapeutic cells may be macroencapsulated or microencapsulated. In accordance with one embodiment of the present invention and with reference to FIGS. 12A-15, living cells (e.g., islet cells) are macroencapsulated in a polyethylene glycol (PEG) based hydrogel construct 54 (see FIG. 33) for insertion into and retention within the inner lumen 23 of the cell container 22. The hydrogel construct 54, in one embodiment, has a generally hollow cylindrical configuration. By encapsulating the cells in a PEG-based hydrogel construct 54, the cells are allowed to transport desired substances, such as glucose, insulin, oxygen, etc., while undesirable substances, such as immune response cells, are kept out of the inner lumen 23 and away from the cells. In embodiments and applications where it becomes difficult to protect the cells against such materials, the hydrogel 54 may be configured to allow the permeation of supplied immunosuppressive agents in order to protect against cell-damaging agents.

In embodiments in which the cell barrier 24 is removed after vascularization, the PEG-based hydrogel construct 54 effectively becomes the new cell barrier and continues to prevent substantial formation of blood vessels within the inner lumen 23 of the container 22 after the cell barrier 24 is removed.

The PEG-based hydrogel construct 54 has beneficial structural properties. The construct is non-degradable and mechanically stable over the envisaged implant period, which improves the viability of the transplanted cells and permits easy removal in cases of emergency or in routine replacement without disrupting the vasculature.

Various methods are known in the art for encapsulating cells in various polymer or gel-based constructs. Therapeutic cells may be macroencapsulated or microencapsulated. U.S. Pat. No. 5,334,640 to Desai et al., hereby incorporated by reference in its entirety, discloses crosslinked biocompatible compositions with an ionically crosslinked component and a covalently crosslinked component for encapsulating biologics with alginate and PEG-based macrocapsules. U.S. Pat. No. 5,976,780 to Shaw, hereby incorporated by reference in its entirety, discloses a macroencapsulation device to encapsulate cells in alginate inside polysulphone hollow fibers. U.S. Pat. No. 6,911,227 and U.S. Publication 2004/0195710 to Hubbell et al., hereby incorporated by reference in their entirety, discloses a method for PEG-based cell microencapsulation with photoinitiation. Sawhney et al. hereby incorporated by reference in its entirety, discloses visible-light-initiated interfacial photopolymerization of multifunctional PEG-base macromers directly upon the surface of islets of Langerhans, producing a conformal barrier hydrogel coating with a thickness of about 10 microns (Sawhney et al., Biotechnol Bioeng 44:383-386, 1994). U.S. Pat. No. 5,879,709 to Soon-Shiong et al., hereby incorporated by reference in its entirety, discloses a method of treating diabetes by implantation of microencapsulated islet cells embedded in macroencapsules with PEG and alginate based hydrogels. International Publication No. WO 03/040235 to Luetolf et al., hereby incorporated by reference in its entirety, discloses methods for improving PEG-based polymer matrix properties with regard to cell infiltration (e.g., immune cells). Balamurugan et al., hereby incorporated by reference in its entirety, discloses an agarose-based encapsulation construct inserted into a prevascularized pouch (Balamurugan et al, “Bioartificial Pancreas Transplantation at Prevascularized Intermuscular Space: Effect of Angiogenesis Induction on Islet Survival,”. Pancreas, Vol. 26, No. 3, pp. 279-85. It is contemplated that any one of the above referenced methodologies may be utilized in the context of the present invention, as will be appreciated by those of ordinary skill in the art.

As noted above, therapeutic cells may be macroencapsulated or microencapsulated. If the cells are macroencapsulated, islet spacing (packing density) can readily be controlled by molding techniques, and it may be easier to insert islets in one gel formation (as a pre-form) rather than injecting multiple beads, as in the case of microencapsulation. Additionally, macroencapsulation allows for better control of cell placement and proximity to blood vessels and better prevents ingress of blood vessel formation into the lumen 23 of the cell container 22.

The following discussion makes reference to an underhydrated hydrogel construct, which can be referred to as a “swelling” hydrogel, with respect to the PEG-based cell construct. The discussion also refers to a substantially fully hydrated hydrogel construct, which can be referred to as a “non-swelling” hydrogel, with respect to the agarose-based cell construct. In a swelling hydrogel, the hydrogel is underhydrated prior to insertion into the permeable container. Being underhydrated, the hydrogel assumes a smaller size, thus facilitating its insertion into the container. After insertion into the container, the underhydrated hydrogel is hydrated, thus causing it to expand to a desired, larger size. A non-swelling hydrogel will essentially remain the same size after insertion, unless it is mechanically expanded.

It will be appreciated by those skilled in the art that any suitable hydrogel construct may be configured as swelling or non-swelling. Thus, the construct insertion methods described herein, though made with reference to a swelling PEG-based hydrogel and a non-swelling agarose-based hydrogel, respectively, may be considered to be applicable to any swelling hydrogel construct and any non-swelling hydrogel construct, respectively. For example, an agarose-based hydrogel construct may be made to be swelling, and a PEG-based hydrogel construct may be made to be non-swelling.

In one embodiment, cells may be encapsulated in a hydrogel prepared from an 8-arm, 20,000 g/mol PEG functionalized with vinyl sulfone groups, and crosslinked with dithioerythritol (DTT) as described in detail in Example 3. Briefly, cells to be encapsulated were concentrated by centrifugation to form a cell pellet and remove excess surrounding medium. Meanwhile, PEG was functionalized with vinyl sulfone and then mixed with a solution of “RGD peptide” (solution containing peptides having at least the amino acid sequence Arginine(R)-Glycine(G)-Aspartic acid (D)) to add pendant groups to improve the adhesion of cells in the otherwise non-adhesive PEGs, and optionally to provide thiol groups to attach the peptide to the functionalized PEG). The pelleted cells were then resuspended in buffer and mixed with the PEG-VS-RGD mixture. A crosslinker (dithiothreitol, DTT) was added to the previous mixture (cells suspended in PGS-VS-RGD) and the mixture was quickly injected into a mold. After a few minutes in the mold, crosslinking is complete and the PEG-based hydrogel (nominal PEG concentration approximately 10% m/v) has “set” such that the cells are encapsulated in the hydrogel in a shape determined by the mold.

Non-limiting examples of molds suitable for use in accordance with the present invention are provided at FIGS. 12A-C.

FIGS. 12A and 12B illustrate a mold apparatus 1200 and process shown for use with a swelling hydro gel 54, such as a PEG-based hydrogel, although it should be appreciated that such an apparatus and process may be utilized for a non-swelling hydrogel 64, such as an agarose-based hydrogel. As shown, the mold includes a block 1210 (half of the mold block is shown in FIG. 12B) that may comprise a single block with a plurality of cylindrical bores 1202 extending therethrough, or the block may comprise two or more parts that form a plurality of bores 1202 when the parts are connected.

The steps (1) through (7) of the hydrogel molding process are shown in FIG. 12A. In step (1), a lower centering ring 1206 and rod 1224 (or guidewire) are inserted into an empty bore 1222. One of skill in the art will appreciate that the lower centering ring 1226 and/or rod 1224 may alternatively be permanent fixtures in the mold 1220. Then, in step (2), the hydrogel and cell (e.g., islet cells) mixture 54 in liquid form is poured into the bore 1222. A tubular elongated mandrel 1256 is then disposed around the rod 1224 in step (3) and an upper centering ring 1228 is placed around the mandrel 1256. In step (4), the hydrogel 54 is formed into its solid or gel form. In step (5), the mandrel 1256 and hydrogel construct 54 are removed from the bore 1202 and a stop structure 1258 may be disposed at an end of the mandrel 1256 for aid in inserting the hydrogel 54 into the porous container, as will be described below. Step (6) shows the hydration and enlargement of a swelling type hydrogel 54 that takes place once the hydrogel 54 is inserted into the container 22. Step (7) shows the removal of the hydrogel 54 from the mandrel 1256 and stop structure 1258.

FIG. 12B shows the mold 1220 while several hydrogel constructs are being formed. In bore (a), the lower centering ring 1226 occupies the bore in a phase preceding step (1) in the process illustrated in FIG. 12A. In bore (b), the rod 1224 has been inserted, which represents step (1) in the process. Bore (c) contains an apparatus that is in the middle of step (3) in the process. The hydrogel 54 and mandrel 1256 have been inserted into the mold but the upper centering ring 1228 has not yet been placed. Bore (d) shows a hydrogel construct in step (4), in which the hydrogel 54 is being formed into a solid or gel.

FIG. 12C shows a further mold 1250 and process that may be used to form hydrogels in accordance with the present invention. In step (1), a lower centering ring 1257 is inserted into and pushed to the bottom of a glass tube 1252. A rod 1254 (or guidewire) is then inserted into the glass tube 1252 and into a hole 1260 disposed in the center of the lower centering ring 1257. In step (2), a mandrel 1256 is disposed over the rod 1254 and is pushed into the glass tube 1252 until its lower end abuts the lower centering ring 1257. A hydrogel and cell mixture 54 (e.g., islet cells) in liquid from is then poured into the glass tube 1252. An upper centering ring 1228 is then disposed over the mandrel 1256 in order to centrally locate the rod 1254 in the hydrogel mixture 54 and the hydrogel is then allowed to form a solid or gel. In step (4), the hydrogel construct 54, along with the mandrel 1256, rod 1254, and centering rings 1228, 1257 may be removed from the glass tube 1252. In other embodiments, only the mandrel 1256, hydrogel 54, and upper centering ring 1228 are removed from the glass tube 1252. It is appreciated that any elements may remain or be removed from the glass tube 1252 as the situation requires or is desirable. Step (5) shows the hydrogel 54 and the mandrel 1256 ready for insertion into a cell container 22. In other embodiments, the centering rings 1228, 1257 may remain disposed over the mandrel 1256 during insertion of the hydrogel 54 into the container 22.

One of skill in the art can select and adapt methods of forming hydrogels suitable for particular applications. Types of PEG suitable for forming hydrogels for use in accordance with the present invention have properties including, but not limited to, having 2, 3, 4, 5, 6, 7, 8, 9, 10, or more arms and having a molecular mass of about 2000, 5000, 10000, 15000, 20000, or 30000 Da. PEG solution concentrations can be between about 4% to about 50%, preferably about 5-20%, more preferably about 8-15%, including solutions containing about 9% PEG, about 10% PEG, about 11% PEG, about 12% PEG, about 13% PEG, or about 14% PEG. Functional groups added to PEG arms (in particular, appended to the extremities of the arms), include, but are not limited to, alpha-beta unsaturated endgroups that will react in a Michael-type addition reaction with the pendant group and/or thiol crosslinker. Suitable crosslinkers include, but are not limited to, thiol-ended functional groups (R—(SH)ₘ, where m is an integer), e.g., dithiothreitol (DTT, Cleland's reagent), dithioerythritol (DTE), or PEG thiols or sulfides (PEG-SH)ₘ). Suitable crosslinkers may be non-degradable or degradable, e.g., degradable peptide crosslinkers such as enzymatically degradable peptides disclosed in International Publication No. WO 00/44808 to Hubbell et al, hereby incorporated by reference in its entirety. Pendant groups, generally added to improve the adhesion of cells in or to otherwise largely non-adhesive PEGs, include but are not limited to, an “RGD peptide” having the sequence GCGYGRGDSPG (SEQ ID NO: 1), where RGDSP provides adhesive function, and the C (cysteine) contains the thiol needed to attach the peptide to the functionalized PEG (e.g. to the vinyl sulfone group of PEG-₈VS as described above). Other polymers suitable for forming hydrogels in accordance with the present invention include, but are not limited to: synthetic polymers such as polyacrylamide, polyacrylic acid, polyacrylamide-co-acrylic acid, polyvinyl alcohol, polyethyleneimine), polyvinyl pyrrolidone, polyhydroxyethyl methacrylate and analogues of these synthetic polymers; and naturally occurring polymers such as collagen, alginate, or agarose.

To insert the encapsulated cell hydrogel construct 54 into the inner lumen 23 of the vascularized cell container 22, the hollow cylindrical hydrogel construct 54 is disposed over a cylindrical mandrel 56, as shown in FIG. 12D. In one embodiment, as can be appreciated from the previous discussions, the hydrogel 54 may be made having a water content below equilibrium and be underhydrated slightly so that its outer and inner diameters are temporary reduced, thus forming a gap 71 between the outer cylindrical surface of hydrogel construct 54 and inner cylindrical surface 22a of the container 22, and allowing for easier and less obstructive insertion into the cell container 22. The mandrel 56 with the reduced diameter hydrogel 54 is pushed into the feeder tube 32 and into the inner lumen 23 of the cell container 22 until it reaches the end plug 28. The mandrel 56 is formed of a rigid material, such as metal or plastic, so that when it reaches the end plug 28 it will encounter resistance and prevent longitudinal deformation of the hydrogel construct 54.

The hydrogel 54, once inserted into the cell container 22, will then swell under physiological conditions to fill the space 23 within the container 22 as it absorbs water to achieve equilibrium. Alternatively or in addition, fluids may be supplied in order to cause the underhydrated hydrogel 54 to swell by rehydration. When the hydrogel 54 expands, its inner and outer diameters will expand, as shown in FIG. 13. As a result, the hydrogel construct 54 will detach from the mandrel 56 and expand to abut against the interior wall 22a of the container 22, thereby bringing the encapsulated cell construct 54 in close proximity to the vasculature (or contact with the vasculature when there is no cell barrier 24). The mandrel 56 may then be removed from the device.

In order to facilitate the removal of the mandrel 56, as shown in FIGS. 12D and 13, a tubular (cylindrical) stop structure 58 may be disposed over the cylindrical mandrel 56. The stop structure 58 may be a ratchet device and may include a radially outwardly extending annular flange 59 at a rearward end thereof. The stop structure 58 and flange 59 are used to prevent over-insertion of the hydrogel 54 as a result of the flange 59 abutting against the rearward end of feeder tube 32, as shown in FIG. 13. The ratchet or stop structure 58 may also be used to aid in removing the mandrel 56, without removing the hydrogel 54, by the forward end of the stop structure 58 abutting the proximate end of the hydrogel 54 while the mandrel 56 is pulled out. The stop structure 58 keeps the hydrogel 54 in the proper position within the cell container 22 as the mandrel is slid out from within the hydrogel construct 54. The tube or stop structure 58 may then be removed, leaving only the hydrogel construct 54 within the container assembly 150, as shown in FIG. 14.

In another embodiment, rather than providing an underhydrated hydrogel and subsequently hydrating it, a mechanical expansion can be used to expand a radially undersized hydrogel construct into contact with the surface 22a. For example, a radially expandable mandrel can be used to achieve this. In another embodiment, the PEG-based hydrogel construct 54 is not underhydrated and is formed to closely match the configuration of the interior of the container 22 prior to insertion.

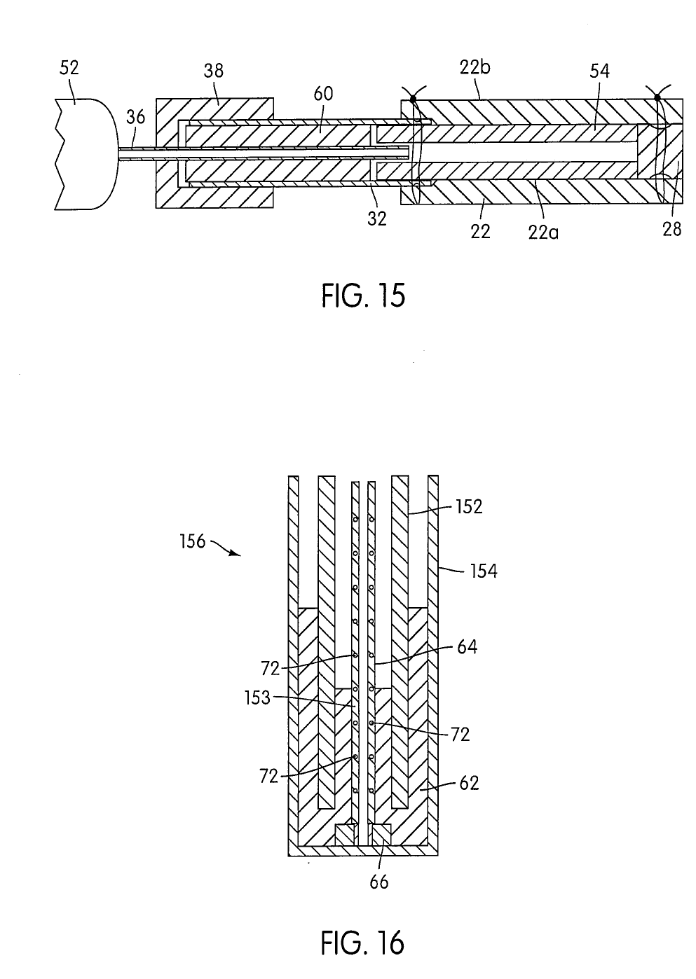

The pump 52 and catheter 36 for supplying nutrients or other materials to the hydrogel construct are then attached to the feeder tube 32, as shown in FIG. 15. A second cored plug 60 having a slightly larger diameter than the first cored plug 34 (used during the vascularization step; the second cored plug 60 is larger due to the absence of the barrier support 26 in the transplantation step) may be used to centrally locate and retain the catheter 36 within the device. The cored plug 60 also functions to occupy space within the feeder tube 32 to prevent ingress of liquids from the outside through the catheter feeder tube 32 into the device, and reduces volume of fluid that may need to be provided to or suctioned out of the device.

In an alternative embodiment, a single cored plug may be used in both the vascularization and the transplantation processes. An example of a suitable plug for this purpose may be one that is tapered so that each of the different diameters may be accommodated by simply varying the distance the plug is inserted into the device.

The end cap 38 may be fastened to the feeder tube 32 in a similar manner discussed above in order to enclose the device and to provide further support for the catheter 36. At this point, the implanted device 10 is ready to provide therapeutic aid to the patient. Methods and procedures to maintain and improve the performance of the cell therapy device 10 are discussed below.

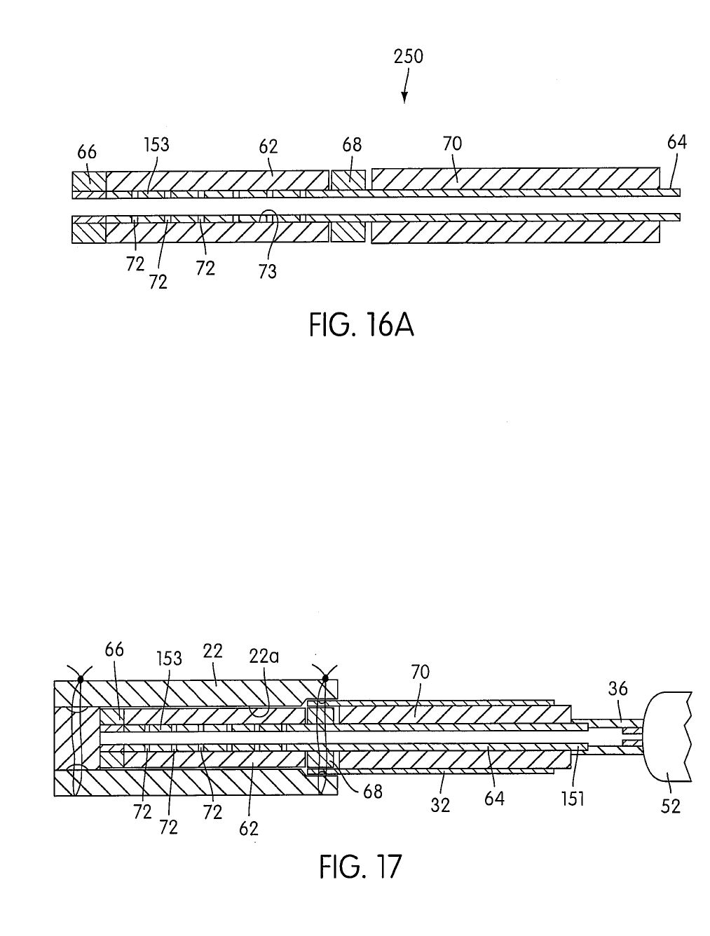

In accordance with another embodiment of the present invention, as illustrated in FIGS. 16-19, an encapsulated cell construct 62 is formed of, for example, an agarose-based hydrogel. In other embodiments, hydrogels may be based upon one or more various materials including, but not limited to, PEG, alginate, chitosan, collagen, matrigel, or polyacrylamide. Since agarose is a natural saccharide polymer derived from seaweed, it has advantages in terms of a presumed cell bio-compatibility. However, as compared to the PEG-based hydrogel 54, an agarose-based hydrogel 62 has relatively weaker structural properties. Accordingly, a different insertion and retention mechanism may be used for an exemplary agarose-based hydrogel 62 than the PEG-based hydrogel 54. It is appreciated that while an agarose-based hydrogel and method for insertion are described herein, the method of formation and insertion may be applied to any suitable hydrogel such as those mentioned above or any other hydrogel.

As can be appreciated from FIG. 16, an agarose-based encapsulated cell hydrogel formation 62 is prepared using a syringe assembly 156. As shown in FIG. 16, agarose 62 in a container 154 is drawn up a glass tube 152 and onto a support tube 64, which may be provided with a lower perforated wall portion 153. The glass tube 152 is then pushed down onto the washer 66 to seal and protect the hydrogel 62. The glass tube 152 may then be removed and allowed to gel by cooling briefly to about 0 to 4 degrees Celsius. By this method, a uniform hydrogel formation is formed due to the abilities of the glass tube 152 to contain and form the agarose 62 as it is drawn onto the support tube 64. In an alternative method for forming the hydrogel, a hydrogel and cell mixture in liquid form may be directly inserted into a mold, such as a glass tube 152 as described above, with a pipet or syringe, sealed with a washer, and allowed to gel by cooling.

With reference to FIG. 16A, an agarose-based hydrogel assembly 250 is shown. This assembly 250 includes the agarose-based hydrogel 62, the hydrogel support tube 64, washers 66 and 68, and an optional elongated cored plug 70. The islet containing agarose-based hydrogel 62, in one embodiment, has a generally hollow cylindrical configuration. As noted above, the hydrogel support tube 64 may be configured to have a perforated portion 153, having perforations 72, that is in contact with an inner cylindrical surface 73 of the hydrogel 62. The hydrogel support tube 64 further comprises a distal washer 66 and a proximal washer 68, disposed adjacent to respective opposite ends of the hydrogel 62. The washers 66, 68 have an outer diameter of essentially the same dimension as the outer diameter of the hydrogel 62, or in another embodiment, a slightly larger outer diameter than the outer diameter of the hydrogel 62.

After vascularization and removal of the pump 52, catheter 36, and cell barrier assembly 125 as described above, the agarose-based hydrogel assembly 250 is inserted into the inner lumen 23 of the container 22 via the feeder tube 32 (See FIG. 17). In this embodiment, each of the washers 66, 68 and the hydrogel formation 62 are sized to fit the inner lumen 23 of container 22 prior to insertion (as opposed to undersizing and swelling as is done with the PEG-based hydrogel 54). Specifically, for example, the outer diameters of the washers 66, 68 and the hydrogel formation 62 are only slightly less than the diameter of inner surface 22a defining the diameter of lumen 23 or, if the vasculature is disposed in the lumen 23 (inwardly beyond the inner surface 22a), then the outer diameter of washers 66, 68 and hydrogel formation 62 are slightly less than the inner diameter formed by the surface of the vasculature. As the agarose-based hydrogel assembly 250 is inserted into the container 22, the washers 66, 68 serve as a shield or protective structure that prevents or reduces interaction or contact between the hydrogel 62 and the interior wall 22a of the container 22 so that the hydrogel 62 is not deformed during insertion. The washers 66, 68 therefore ensure that the hydrogel 62 substantially maintains an even distribution of encapsulated cells along the longitudinal length of the inner lumen 23, thereby enhancing cell efficiency and function.

As seen in FIGS. 16A and 17, the cored plug 70 or other stoppage device may be disposed over the support tube 64 prior to insertion of the hydrogel assembly 62. Alternatively, the cored plug 70 may be inserted into the feeder 32 and onto the support tube 64 after insertion of the hydrogel formation 62 into the container 22. The cored plug may, in some embodiments, be a tapered plug and may be the same cored plug that was used during the vascularization process.

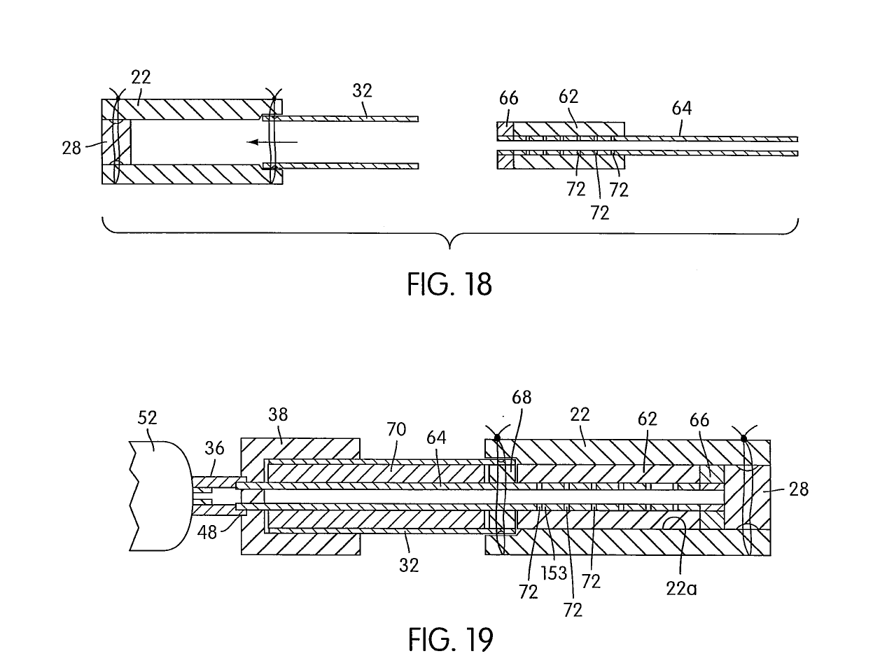

In another embodiment, shown in FIG. 18, only a distal washer 66 is provided adjacent to the hydrogel construct 62 along the support tube 64. If a cored plug 70 is used, it would extend all the way to the hydrogel construct 62 (i.e., the proximate washer 68 would not be present). The cored plug 70 has generally the same function and purpose as cored plug 60 discussed above (e.g., see discussion of FIG. 15).

As seen in FIG. 17, pump 52 is re-attached for communication with the container 22. Specifically, the pump 52 may be attached to tube or catheter 36 and to a proximal imperforate end portion 151 of the hydrogel support tube 64 for delivery of optional agents and materials, as discussed below. Alternatively, the catheter 36 may be attached to the support tube 64 via the inner surface of the support tube 64 (i.e., a friction fit between the inner diameter of support tube 64 and outer diameter of catheter 36), or the support tube 64 may be attached directly to the pump 52, with no intervening catheter 36. Since the support tube 64 can remain within the implanted device during therapy, the imperforate end portion 151 of the support tube 64 enables supplied materials and agents to pass through the support tube 64 to reach the perforated end portion 153 and through perforations 72 to reach the hydrogel construct 62 and, in some cases, to the vasculature and surrounding tissue in and around the cell container 22. In one embodiment, only the end portion 153 of the support tube 64 that carries the hydrogel 62 has perforations 72 so as to minimize leakage and/or waste of supplied materials to other areas of the device 10. Although pump 52 is described and illustrated as the same pump that was used in the vascularization stage, it is recognized that a different pump may be used, such as one having specific characteristics that are more appropriate for dispensing nutrients or other agents than for vascularization growth factors.

As shown in FIG. 19, in one embodiment, end cap 38 may be reconnected to the feeder tube 32 to seal the device. In this case, the hydrogel support tube 64 may pass through the end cap bore 48 to be connected to the pump 52 via tube or catheter 36. Other arrangements and configurations may become apparent to those skilled in the art without departing from the scope of the invention. For example, the catheter 36 may instead be configured to be inserted into the bore 48 and connect to the support tube 64 within the enclosed device.

In one embodiment, the agarose-based hydrogel construct 62 may be underhydrated prior to insertion and inserted into the container 22 in a manner similar to that described with respect to the underhydrated PEG-based hydrogel construct 54.

In an embodiment, living cells that produce (translate and secrete) therapeutic proteins may be transplanted inside, for example, the cell container 22. The transplanted cells may be autologous in nature and derived from readily accessible sources, such as platelet-rich plasma (PRP), autologous platelet gel (APG), bone marrow, or adipose tissue. Alternatively, the cells may be from allogeneic or xenogenic sources, such as umbilical cord blood or porcine tissue. The translation and/or secretion of the therapeutic protein by the transplanted cells with be affected by genetic engineering (in vitro or in vivo transfection) and/or simulating factors by a delivery system.

The secretion of the therapeutic protein in vivo may be controlled by electrical or chemical simulation. In an embodiment, the secretion of the therapeutic protein may be controlled by optical activation of cytochrome oxidase, which is a mitochondrial enzyme that is normally regulated via the binding of carbon monoxide and/or nitrous oxide. An increase in cytochrome oxidase acivity subsequently increases the adenosine 5′-triphosphate (ATP) to adenosine diphosphate (ADP) ratio. Specifically, the rate of insulin secretion from transplanted pancreatic islets or beta-cells may be controlled through the optical activation of cytochrome oxidase. Illumination of cytochrome oxidase with the proper wavelength of light, e.g. 670 nm, may activate the enzyme, which may in turn affect the rate (e.g., increase) of the secretion of the therapeutic proteins. The light may be delivered to the device 10 from an external or implanted device or fiber optic cable. In an embodiment, blue light (i.e. light having a wavelength of about 450-495 nm) may be used to decrease the rate of secreation.

G. Maintenance of Cell Therapy Device

As discussed herein the cell therapy device may be maintained by providing the encapsulated therapeutic cells with various agents such as nutrients, metabolites, immunosuppressive agents, and other agents as determined by one of skill in the art. In the case of both the PEG-based hydrogel construct 54 and the agarose-based hydrogel construct 62, the pump 52 and catheter 36 supply the encapsulated therapeutic cells with such agents etc. by delivering the agents to the container 22, and in particular, by delivering agents to the inner lumen or void space 23 within the tubular hydrogel construct 54, 62, as shown in FIGS. 15 and 19.

Pumps may be implanted into the patient, or may be external (outside the patient's body cavity). In one embodiment, the pump 52 may be implanted into the patient in the vicinity of the therapy device 10 and is connected thereto by the catheter tube 36. Non-limiting examples of various configurations that may be used for pump 52 are described herein. It should be appreciated that the pump 52, in various embodiments, can be charged or filled with different agents. In certain embodiments, upon depletion, the pump 52 can be re-charged or refilled without having to remove the pump 52 from the patient.

Examples of the types of pumps that can be used with the present invention include, but are not limited to, osmotic pumps such as Alzet® pumps (DURECT Corp., Cupertino Calif.), electro-osmotic pumps such as EKPump® devices (Eksigent Technologies LLC, Dublin Calif.), gas-generating cells such as Microlin Micro Pumps (Microlin Technologies, Ceramatec Inc., Salt Lake City Utah), microfludic pumps, peristaltic pumps such as Synchromed® pumps (Medtronic Inc., Minneapolis Minn.), syringe/piston pumps, e.g., Duros™ (Eldex Laboratories, Inc., Napa Calif.) or MiniMed® pumps (Medtronic Inc., Minneapolis Minn.), diaphragm pumps, centrifugal pumps, various positive displacement pumps, auger/screw pumps, constant pressure pumps such IsoMed® pumps (Medtronic Inc., Minneapolis Minn.), gravity/head pumps, and pumps driven by microchip technologies. In accordance with one embodiment, the pump 52 may be an osmotic, electro-osmotic, or programmable pump. Electro-osmotic pumps may be advantageous for use in the device due to their small size, minimal invasiveness, relative inexpensiveness, and amenability to control and shut-down. In accordance with another embodiment, the pump 52 may be an infusion pumping apparatus such as a syringe or piston pump, and may be external, internal (implanted), programmable, refillable, or may have other desirable features. Additional examples of some pumps that can be used with the present invention are disclosed in U.S. Pat. Nos. 4,685,903; 4,562,751; 6,248,093; 6,554,798; 6,585,695; 6,997,911; 6,770,067; 6,595,756; and 6,932,584, the entire contents of each patent being hereby incorporated by reference in their entirety.

Other pumps that can be used are peristaltic pumps, and pumps that are capable of providing agents at a variable and/or programmable rate. Additional specific pumps that can be used are disclosed in the following U.S. Patents, the entire contents of which are hereby incorporated by reference: U.S. Pat. Nos. 7,122,026; 7,072,802; 7,066,915; 7,001,359; 6,685,452; 6,423,029; 6,360,784; 6,036,459; 4,692,147; 4,576,556. Other pumps that may be used are implantable, gas-powered bellows pumps. Specific pumps that can be used are disclosed in U.S. Pat. Nos. 5,382,236 and 5,395,324, the entire contents of which are hereby incorporated by reference.

While any of these pumps can be used with the device as disclosed herein, the list provided above should not be considered to be limiting in any way. Indeed, numerous different types of pumps can be used, as will be appreciated by those of skill in the art, and those discussed herein are only a few examples of the different types of pumps that are suitable.

Moreover, the present invention further includes devices that do not utilize a pump. In accordance with one embodiment, rather than using a pump, a refillable reservoir can be periodically filled with appropriate agents. Such filling may be accomplished by injecting agents into a subdermally implanted reservoir or container, e.g., using a syringe to inject the agent(s) into the subdermally implanted reservoir or container. Alternately, a refillable container may be mounted externally to the patient for easy refilling. Alternately, an implanted reservoir may be removed from the patient for periodic filling. In accordance with other embodiments, agents can be delivered by polymeric delivery, in situ dissolution of material containing the agent(s), or in situ generation of an agent, e.g., in situ chemical generation of oxygen using a Microlin device (Microlin Technologies, Ceramatec Inc., Salt Lake City Utah), or by mixing a peroxide and an enzyme. It can thus be appreciate that any mechanism for providing agents to the container 22 may be employed.

In any case, the region of the device described herein that contains or otherwise provides or pumps growth factors, oxygen, or other agents to the container is referred to herein as a “source” or “agent source.” A pump or refillable pump is one type of agent source. It should be appreciated that the “agents” referred to herein can be a liquid, gaseous, gel-like, or solid form, or any combination thereof, depending on the particular embodiment and particular application.

In addition, while the pump or agent source disclosed herein can be connected with the porous or permeable container via a catheter or other conduit, in some embodiments the agent source or pump may be immediately connected with the porous or permeable container, with no intermediate conduit or catheter. In another embodiment, the agent source or pump may be formed within or considered to be part of (or integral with) the container.

As noted previously, the pump 52 is suitable for providing various agents to the hydrogel constructs 54 or 62, such as immuno-suppressive agents. While it is envisioned that the hydrogel construct 54, 62 effectively restricts the passage of immune cells to the encapsulated therapeutic cells (e.g., islets), it may be possible that the therapeutic cells remain vulnerable. Accordingly, immuno-suppressive agents are delivered locally so as to protect the therapeutic cells. Such immuno-suppressive agents may include, but are not limited to, one or more of the following: Humira®, a recombinant human MAb from phage display (Abbott Laboratories), Enbrel®, a human TNF-α receptor fused to human Ab constant region (Amgen), Remicade®, a chimeric anti-TNF-α MAb (Centocor), and NEKTAR®, a chimeric anti-TNF-α F_{ab} Ab fragment conjugated to PEG (Celltech), mycophenolate moefetil (MMF, CellCept®), mycophenolate morpholinoethyl, mycophenolic acid, cyclosporine (Sandimmune® and NEoral®), corticosteroids, azathiaprine (also known as Imuran®), chlorambucil (Leukeran®), cyclophosphamide (Cytoxan® and Fludara®), rapamycin (sirolimus, Rapamune®), tacrolimus (FK506), anhydrous tacrolimus, tacrolimus monohydrate, cytarabine, mercaptopurine, inactivating ligands (e.g., CTLA4Ig), immuno-suppressive polyclonal antibodies (e.g., anti-lymphocyte serum (ALS), antilymphocyte immunoglobulin (ALG), antithymocyte immunoglobulin (ATG), antilymphocyte serum, antithymocyte serum, lymphocytic antiserum, thymitic antiserum, and ATGAM (lymphocyte-selective immuno-suppressant)), anti-CD3 antibodies (e.g., Muromonab-CD3), anti-CD25 antibodies, anti-CD40 ligand antibody, anti-CD40 antibody, anti-CD30 antibody, anti-OX40 antibody, and anti-CD28 antibody (see, e.g., U.S. Patent Application No 2002/0006403 A1), anti-cytokine antibodies (e.g., anti-TNF antibodies and anti-IFN antibodies), azathioprine or azathioprine sodium, basiliximab, cyclosporin or cyclosporine (cyclosporin A), daclizumab (dacliximab), glatiramer or glatiramer acetate, sirolimus, interferon α-2a, recombinant interferon α (rIFN-A or IFLrA), brequinar, brequinar sodium, cyclophosphamide, cyclophosphamide monohydrate, anhydrous cyclophosphamide, dactinomycin, actinomycin C, actinomycin D, meractinomycin, daunorubicin, daunorubicin hydrochloride, daunomycin hydrochloride, rubidomycin hydrochloride, doxorubicin, doxorubicin hydrochloride, adriamycin, adriamycin hydrochloride, fluorouracil, gusperimus, gusperimus hydrochloride, inolimomab, leflunomide, mercaptopurine, mercaptopurine monohydrate, purinethiol, anhydrous mercaptopurine, methotrexate, methotrexate sodium, methotrexate disodium, alpha-methopterin, amethopterin; mustine, mustine hydrochloride, chlormethine hydrochloride, chlorethazine hydrochloride, mechlorethamine hydrochloride, nitrogen mustard (mustine), mizoribine, vinblastine, vinblastine sulfate, vincaleukoblastine sulphate, capecitabine, carmofur, epirubicin, epirubicin hydrochloride, idarubicin, idarubicin hydrochloride, mafosfamide, menogaril, mitozantrone, mitozantrone hydrochloride, mitoxantrone hydrochloride, pirarubicin, tepirubicin, piroxantrone, piroxantrone hydrochloride, anthrapyrazole hydrochloride, oxantrazole hydrochloride, teloxantrone, teloxantrone hydrochloride, thioguanine, anhydrous thioguanine, thiguanine hemihydrate, tioguanine, trofosfamide, trilophosphamide, trophosphamide, uramustine, uracil mustard, vincristine, vincristine sulphate, leurocristine sulphate, vindesine, vindesine sulfate, desacetyl vinblastine amide sulfate, vinorelbine, vinorelbine tartrate, vinorelbine Bitartrate, zorubicin, zorubicin hydrochloride, rapamycin, thalidomide, clofazimine, fludarabine, guanosine arabinoseide, cytosine arabinoseide, prednisone, glucocorticoids, and a pharmacologically or physiologically acceptable salt of any of the foregoing. It may also be desirable to provide agents that increase or preserve encapsulated cells. Non-cell-type-specific agents such as anti-apoptotic agents or anti-scarring agents, may be provided to promote survival and/or to avoid injury to the encapsulated cells. Cell-type-specific agents to promote survival and/or proliferation of one or more encapsulated cell types, e.g., islet cells. In certain embodiments, encapsulated islet cells may be provided with stimulators of islet β cell proliferation and/or neogenesis, or inhibitors of islet cell death, e.g., as disclosed in Baggio et al., (Ann. Rev. Med. 57:265-2819, 2006), hereby incorporated by reference in its entirety. Suitable agents may include, but are not limited to, glucagon-like peptide-1 (GLP-1), glucose-dependent insulinotropic polypeptide (GIP), dipeptidyl-peptidase-IV (DPP-IV) inhibitors, epidermal growth factor/gastrin, thiazolidinediones (TZDs), growth hormone, hepatocyte growth factor, human placental lactogen, islet neogenesis-associated protein (INGAP), insulin-like growth factors, parathyroid hormone-related peptide, prolactin, keratinocyte growth factor, or betacellulin,

It is understood that agents may be delivered to encapsulated cells by a combination of local delivery of certain agents with systemic delivery of other agents. In a non-limiting embodiment, encapsulated islet cells may be provided with antilymphocyte serum (ALS) by localized delivery through the pump 52 and catheter line 36 as described above, and exposed to exendin-4 (a GLP-1 agonist) by systemic delivery, in a modification of the combination treatment disclosed by Ogawa et al. (Diabetes 53:1700 1705, 2004) hereby incorporated by reference in its entirety. In another non-limiting embodiment, encapsulated cells are provided with TNF-α by localized delivery through the pump 52 and catheter line 136 as described above, while anti-TNF is delivered systemically to eliminate memory T cells activated by the implant, in a modification of the combination treatment disclosed by Kodama et al. (Cell Mol Life Sci 62:1-14, 2005) hereby incorporated by reference in its entirety.

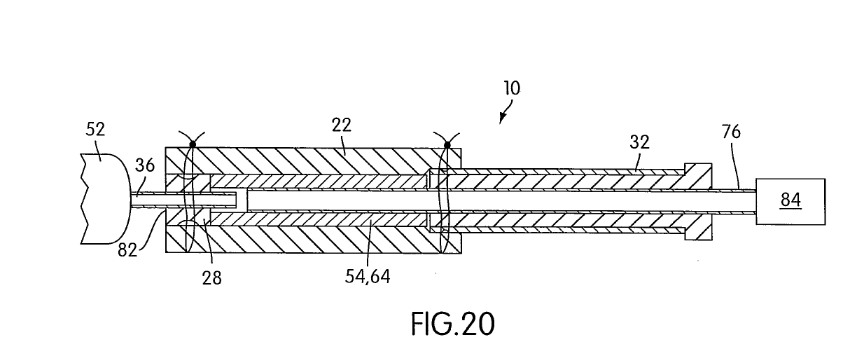

It may also be desirable to provide a supply of oxygen to the encapsulated cells, e.g., encapsulated islet cells. Oxygen supply may be accomplished through the pump 52 and catheter line 36 as described above or through an additional supply line used exclusively for this purpose. As shown in FIG. 20, an oxygen supply line is provided by an oxygen source 84 and supply tube 76 disposed at a side of the container 22 while immuno-suppressive agents or other materials are provided by a pump 52 connected to the opposite side of the container 22 via catheter 36. The opposite configuration, in which agents and other materials are supplied from the feeder tube 32 side of the device 10 and oxygen is supplied from the cored plug 28 side of the device, is also contemplated.

One of skill in the art will appreciate that supply lines 76 and 36 delivering various materials to the inner lumen 23 may be combined or separated as is deemed appropriate for the specific application. Once delivered to the lumen 23, and more particularly to the inner cavity within the hydrogel construct, the various materials and agents delivered through supply lines 76 and 36 are diffused and absorbed into the hydrogel construct to be received by the encapsulated cells.

Further, in other embodiments, oxygen or other agents may be supplied to the cells via the outer surface of the container 22 (allowing the agent to pass through the container 22 to reach the cells). Moreover, one or several agents may be supplied to the inner lumen 23 while another or several other agents may be supplied to the cells via the outer surface of the container 22. In a further embodiment, agents may be delivered to the region between the cell matrix (hydrogel) 54, 62 and the container 22 or directly to the cell matrix 54, 62.

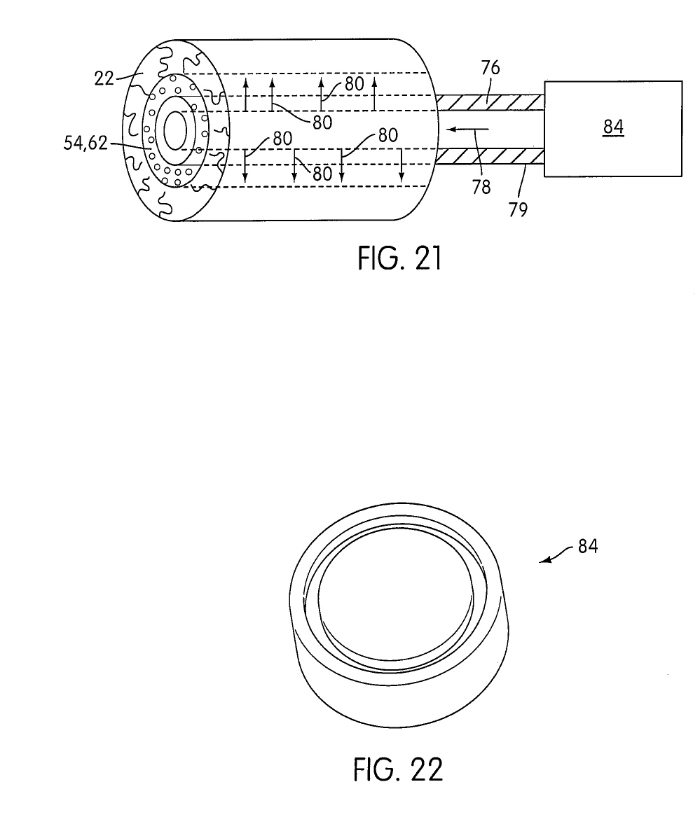

A source of oxygen for the living islet cells may, in one embodiment, take the form of an implantable electro-chemical gas generator. U.S. Pat. No. 5,427,870 to Joshi et al., hereby incorporated by reference in its entirety, discloses a gas releasing electrochemical cell for fluid dispensing applications. Such a device, or a similar device, may be implanted in a patient and connected to the supply tube 76, as illustrated in FIGS. 20 and 21.

As shown in FIG. 22, such an oxygen generating device 84, which is commercially available, is about the size of a watch battery cell and is small enough to be implanted subcutaneously alongside or in proximity of the cell therapy device 10 with minimal patient discomfort. An electro-chemical gas generating cell 84 is additionally advantageous in view of its ability to deliver a precise flow of clean gas. That is, electrochemical cells 84 typically produce a gas that is substantially free of toxic byproducts. It is desirable to specifically configure the flow rate of oxygen to the cells so as to supply enough oxygen to prevent hypoxia but restrict its supply so as to avoid unwanted effects on blood vessel growth and to prevent toxic levels of oxygen. Electro-chemical cells 84 use solid state reactions and are relatively small, making them good candidates for this purpose.

If it is assumed that 1 million islet cells are needed for diabetic therapy in humans, approximately 125 cc of oxygen per day are estimated to be sufficient (at 4 pmol O₂ per islet per minute). Accordingly, a cell having a diameter of about 2.8 cm and a length of about 4 cm would provide sufficient oxygen to meet or exceed all of the islet oxygen needs for about 3 months. One of skill in the art will appreciate that this example is non-limiting. It serves merely to provide a possible combination of requirements to support a particular theoretical drug therapy.

In an embodiment, an oxygenator may be used to supply oxygen to encapsulated cells. A circulating liquid with a high oxygen capacity, such as perfluorocarbon oil (PFC), may be pumped into the oxygenator. Oxygen from the body may passively diffuse through a membrane and into the circulating liquid. The membrane may include any suitable material, such as silicone rubber. By using oxygen that is already in the body, the maximum local oxygen partial pressure should not exceed the levels that would otherwise be found in vivo. The oxygenated fluid may then be pumped into the encapsulation device. The oxygen may be delivered to the encapsulated cells via passive diffusion through a membrane, such as silicone rubber. The oxygen-depleted fluid may be recycled to the oxygenator so that it may be replenished with oxygen.

To estimate the circulation flowrate (Q_{circ} [mL/hr]) that may be needed to supply an arbitrary number of islets (Nᵢₛₗₑₜₛ), a steady-state mass balance for oxygen in an encapsulation device is represented by Equation (1):

Q_{circ}KSP_{oxy}=Q_{circ}KSP_{dev}−NᵢₛₗₑₜₛR_{O}_{₂} (1)

in which losses to and supply from the surrounding tissue and vasculature are neglected. The solubility of oxygen in water is S [mM/mmHg] and the partition coefficient of oxygen in the circulating fluid is K [−]. The oxygen consumption rate is R_{O2} [mol-O₂/Islet-hr] and the partial pressures of fluid leaving the oxygenator and encapsulation device are P_{oxy} and P_{devs}, respectively [mmHg]. Equation (1) can be rearranged to obtain Equation (2):

$${Q}_{clrc} = \frac{{N}_{islets} {R}_{{O}_{2}}}{KS ( {P}_{dev} - {P}_{oxy} )} \qquad ( 2 )$$

Using the parameter values listed in Table 1 below, it is estimated that a circulation rate of about 10-mL/hour would be needed for a device containing about 300 k islets (Nᵢₛₗₑₜₛ=300 k), which may be enough to make a human insulin-independent. It is estimated that approximately a 1-Watt power source should be used to achieve such a circulation rate, which could be supplied by an implantable rechargeable battery system. In an embodiment where water is used as the circulating fluid instead of PFC (K=1 in Equation (2)), the circulation rate would increase 20-fold over the embodiment that uses PFC.

TABLE 1
Parameters used to estimate Q_{circ}.

| Name | Description | Value | Units |
|---|---|---|---|
| K | Partition coefficient of | 20.3 | — |
| | oxygen in PFC (vs. water) | | |
| S | Solubility of oxygen in water | 0.0124 | mM/mmHg |
| R_{O2} | Oxygen consumption rate | 2 × 10⁻¹⁰ | mol-O₂/Islet-hr |
| | | (3.2 × 10⁻¹²) | (mol-O₂/Islet-min) |
| P_{oxy} | Oxygen partial pressure | 40 | mmHg |
| | exiting oxygenator | | |
| P_{dev} | Oxygen partial pressure | 20 | mmHg |
| | exiting encapsulation device | | |

Returning to the embodiment illustrated in FIG. 21, once supplied oxygen reaches the inner lumen 23, it may be desirable to distribute the oxygen evenly to the encapsulated cells so as to reduce the possibility of over- or under-supply of oxygen to the cells. For this purpose, a tubular catheter 76 may be used that has suitable oxygen permeability characteristics, such as one made of silicone, fluorosilicones, hydrogels, etc. As illustrated schematically in FIG. 21, catheter 76 may be made of a highly permeable silicone material that disperses the oxygen across the diffusive surface of the catheter 76 to the therapeutic cells 54, 62. In one embodiment, the silicone tube has an outer diameter of between about 0.7 mm and about 1.1 mm, an inner diameter of between about 0.4 mm and about 0.8 mm, and a wall thickness of between about 0.3 mm and about 0.6 mm.

In one embodiment, the material of catheter 76 can also be used for the catheters 36 described above.

As shown in FIG. 21, the oxygen generator 84 creates a flow of oxygen in a direction indicated by arrow 78 through the catheter 76. It will be appreciated, however, that the gas generator need not be a pure oxygen generator. That is, a mixture of oxygen and other gases may be produced, such as an oxygen-nitrogen mixture. The region of the catheter 76 not in contact with the hydrogel construct 54, 62, may or may not be permeable so that leakage may be prevented if desired. In an embodiment, the region 79 of the catheter 76 between the gas generator 84 and the therapy device may be sealed by an oxygen impermeable material such as a metal or a polymer sleeve in order to prevent the diffusion of oxygen in that region. As gas is supplied to the region of the catheter 76 that is in contact with the hydrogel 54, 62, the oxygen diffuses through the catheter 76 (e.g., silicone), as indicated by arrows 80, and through the hydrogel matrix construct 54, 62 to reach the encapsulated cells, thereby preventing or minimizing the possibility of hypoxia. The oxygen release of the gas generator 84 may be specifically configured to deliver an appropriate level of oxygen so that the islet cells do not receive too much oxygen. The flow of gas from the generator 84 may be controlled by a resistor associated with or within the generator cell. Such a resistor may be used to control the current supplied to the cell, which may control the flow rate of the gas produced by the electro-chemical reaction.

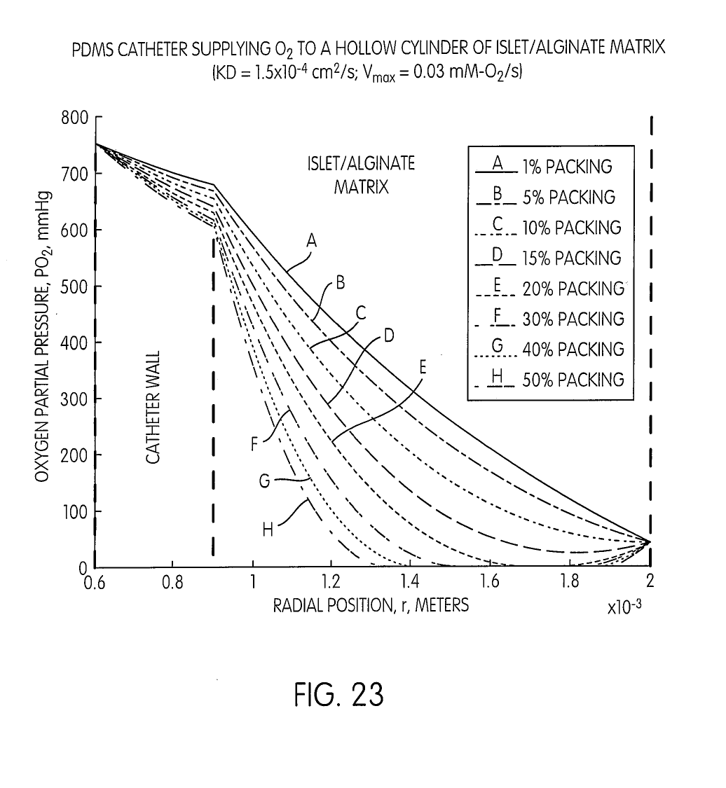

FIG. 23 shows variable oxygen content supply (Y axis) with distance from the center of the silicone catheter. The plot shows the packing densities that can be supported with various oxygen levels as a function of distance from the silicone catheter. From FIG. 23, it can be inferred that as the thickness of the islet-hydrogel layer is increased (reflected by an increase in the radial position), the oxygen level decreases rapidly, particularly for higher packing densities. It can also be inferred from FIG. 23 that the oxygen transport rate decreases with silicone catheter wall thickness. The thickness of the silicone catheter is, in one embodiment, less than about 1 mm, and in another embodiment, less than about 500 microns. The oxygen permeability (KD) of the catheter material in one embodiment less than about 1×10⁻⁵ cm²s⁻¹ (the silicone catheter oxygen permeability is about 1.5×10⁻⁴ cm²s⁻¹). The results shown in FIG. 23 are generated by FEMLAB modeling of volumetric islet consumption (FEMLAB®, COMSOL AB, Stockholm, Sweden.) FEMLAB modeling with a supplied pO₂ level of 140 mm Hg, 1 million islets, 400 microns islet-hydrogel thickness inferred that islet function could be maintained in a cylindrical implant 3 cm in diameter and 15 cm long.

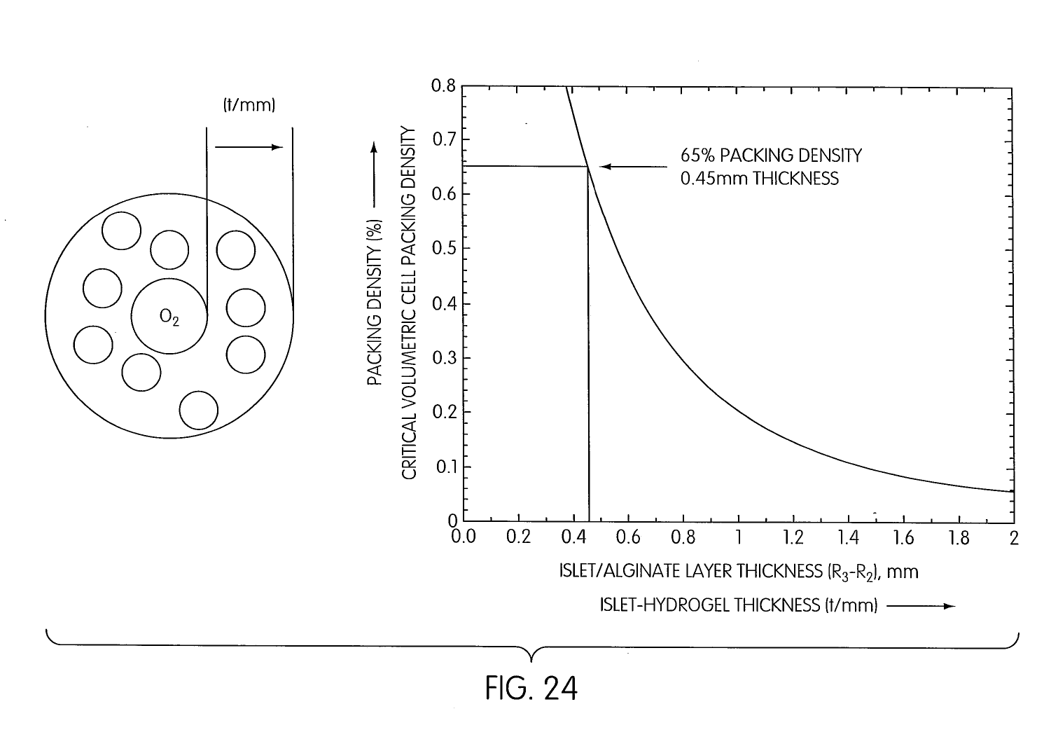

FIG. 24 shows the packing density (Y axis) that can be supported as a function of the islet-hydrogel thickness (distance from the silicone catheter). The diagram to the left of the graph describes how the layer thickness was measured in the model. Pure oxygen (750 mmHg pO₂) is supplied to the silicone catheter. The plot shows that high packing density (65%) can be achieved maintaining islet function with a practical achievable islet-hydrogel thickness of about 450 microns. For one million islets, a cylindrical islet-hydrogel construct for use in a human may have a length of about 8 cm, a diameter of about 2.8 cm, and a thickness of about 400 microns. The islet-hydrogel construct may be even smaller if it is determined that only 300,000 islets are necessary for therapy.

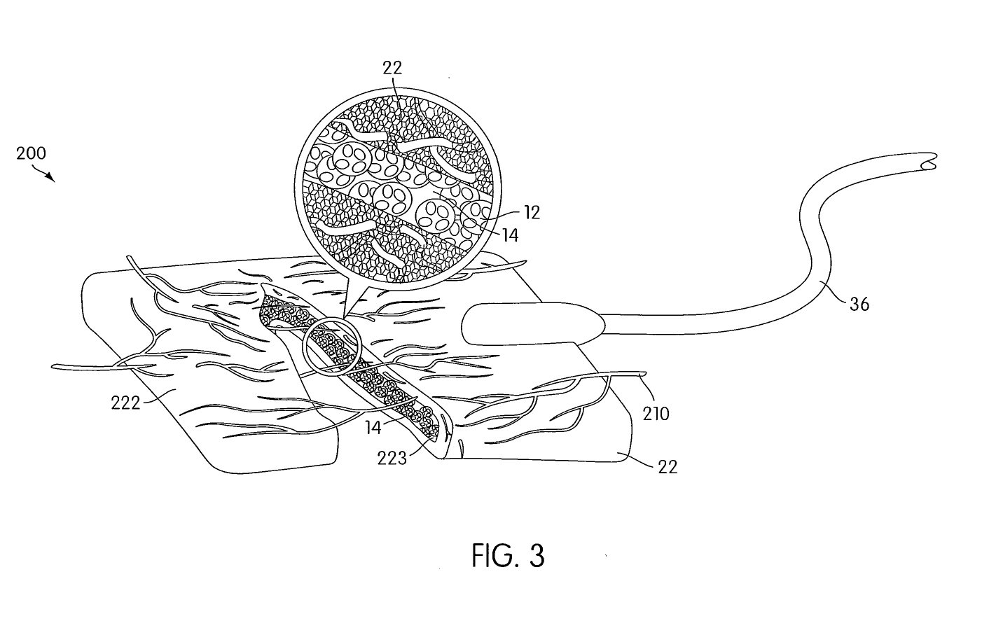

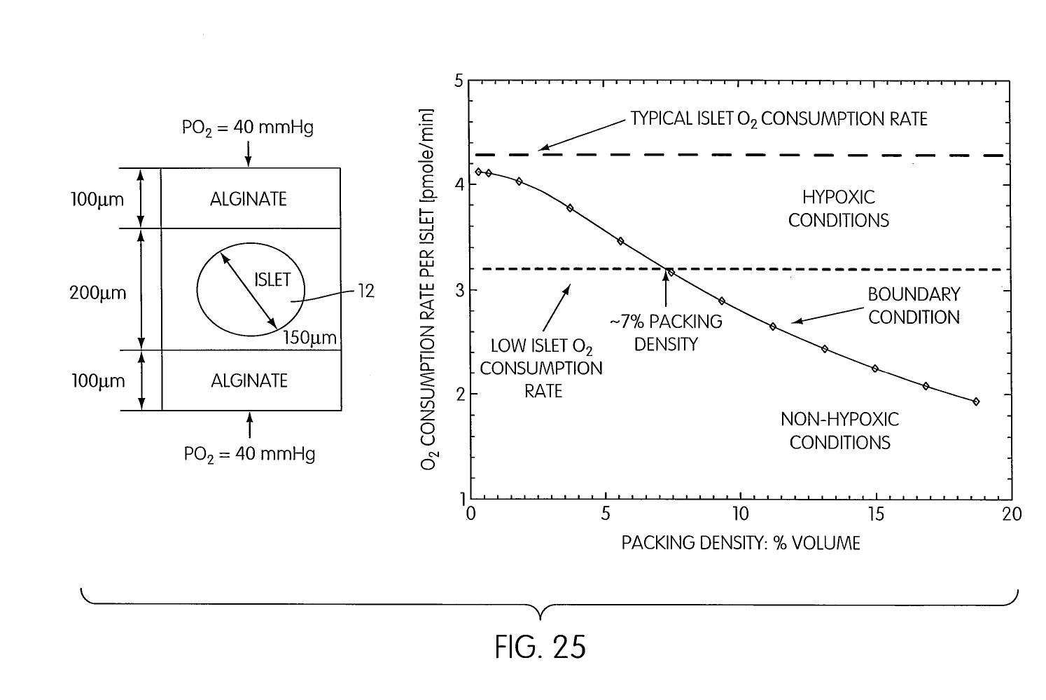

FIG. 25 shows the relevant dimensions of one example of islet cells that can be used in a flat sheet type cell container (as in FIG. 3 or FIG. 4) with the islets close to two outer vascularized surfaces. In this example, the islets have a maximum diameter of 150 microns in a central plane surrounded by water or hydrogel and further encapsulated by hydrogel (e.g., alginate, PEG, etc.) membranes (top and bottom portions) of 100 microns thickness each. The steady-state 3-dimensional diffusion and volumetric consumption (calculated according to Michaelis-Menton kinetics) of oxygen within islet tissue and encapsulation devices was performed using a finite elemental analysis software package (FEMLAB®, COMSOL AB, Stockholm, Sweden). Hypoxia was defined as a condition in which the minimum oxygen partial pressure reached below 5 mm Hg oxygen. This threshold was derived from the literature showing that only 2% of second-phase insulin secretion was observed at a partial pressure of about 5 mm Hg oxygen, compared with 100% at 60 mm Hg partial pressure (Colton et al., Diabetes 42: 12-21 (1993)), which suggests that islets cease to produce insulin when oxygen levels fall below this level. The graph shows predicted operating conditions within the device of hypoxia, non-hypoxia, and the limiting boundary between those conditions, as a function of oxygen consumption per islet (y-axis) and islet packing density (x-axis). As expected, as the distance between neighboring islets decreased (increased packing density), the oxygen consumption level that can be supported by this geometry decreased (e.g., as a function of membrane oxygen permeability, membrane thickness, vascularization level, etc.). Typical oxygen consumption levels for islets reported in the literature are: 4-11 pmol O₂/islet/min (Kennedy et al., Biochem. & Biophys. Research Comm. 259: 331-5 (1999)); 6-14 pmol O₂/islet/min (Longo et al., J. Biol. Chem. 266: 9314-9319 (1991)); 4.5-5 pmol O₂/islet/min (Sweet et al., Diabetes Technol. Therapy 4 (5) (2002)). Accordingly, it may be desirable in the configuration modeled here to provide a supplemental source of oxygen, because the islets may be at least somewhat hypoxic at all packing densities. It understood that the model presented herein is scalable with respect to the level of oxygenation (oxygen supply) sufficient to permit increased packing density of cells and decreased size of the device according to a particular embodiment, such that one of skill in the art may consider the oxygen permeability (flux) of various materials used to supply oxygen to encapsulated cells. In accordance with one non-limiting embodiment, materials having oxygen permeability P of at least about 1000 cm³.mm/M².day.atm, including but not limited to, silicones, fluorosilicones, and hydrogels, may be selected. By way of example, Massey (2003), hereby incorporated by reference in its entirety, discloses the oxygen permeability P, in cm³.mm/M².day.atm, of the following materials: silicone, approximately 19,685 cm³.mm/M².day.atm at 20-25° C.; fluorosilicone, approximately 7599 cm³.mm/M².day.atm at 20-25° C.; styrenic thermoplastic elastomer (TPE), approximately 6831 cm³.mm/M².day.atm at 23° C.; ethylene propylene diene monomer (EPDM) rubber, approximately 1641-1901 cm³.mm/M².day.atm at 23° C.; and acrylic hydrogels (measured as contact lens permeability and units converted), approximately 1500 cm³.mm/M².day.atm at 20-25° C. (Massey, Permeability Properties of Plastics and Elastomers: A Guide to Packaging and Barrier Materials, 2^{nd} Ed., Plastics Design Library, 2003) In contrast, for some applications, fluoropolymer resins, polypropylene, polycarbonate, polystyrene, polyurethane, nylon 6, polyimide, and polyester have oxygen permeabilty P values below 1000 cm³.mm/M².day.atm may be used. The above materials are non-limiting examples.

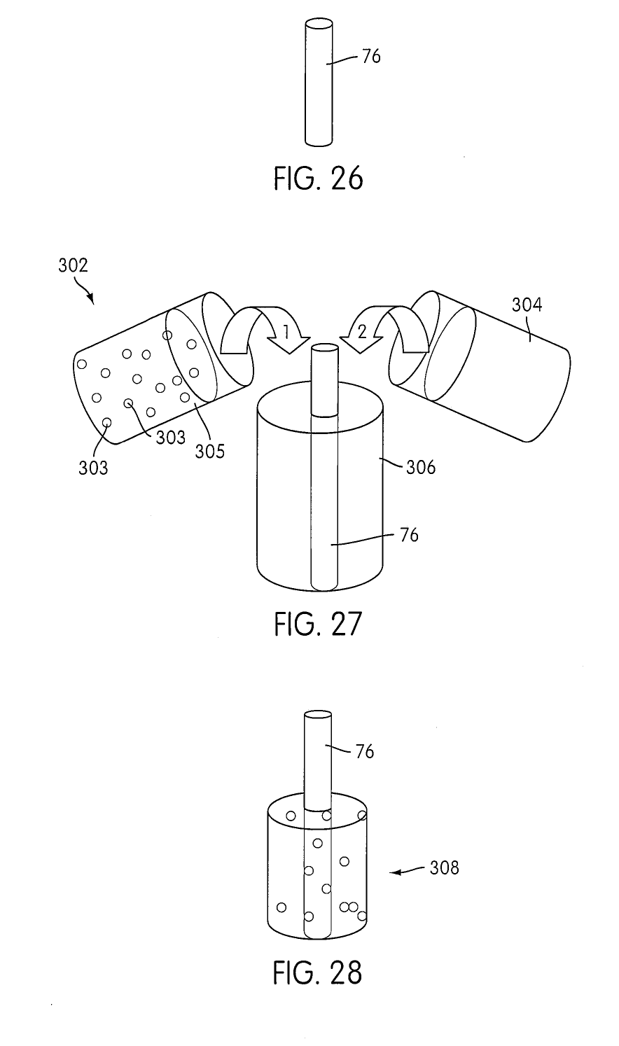

FIGS. 26-31 illustrate an alternative embodiment of the invention in which a cell encapsulation matrix is formed around an oxygen-supplying catheter. In FIG. 26, a silicone catheter 76 is provided, having an outer diameter in the range of between about 1.5 mm to about 2.1 mm and an inner diameter in the range of between about 1.0 mm and about 1.6 mm. As noted above, silicone is chosen because of its high permeability to oxygen, although one of skill in the art will appreciate that other materials may be used, such as fluorosilicones or hydrogels.

In a next step, shown in FIG. 27, an islet 303 and alginate 305 mixture 302 (e.g., with packing density in the range of between about 10% and about 30%) is mixed with strontium chloride (SrCl₂) 304 in a disposable mold 306. The alginate mixture 302 will shrink upon cross-linking, so it may be desirable that the mold be oversized accordingly.

After the alginate mixture 302 cures, it forms an alginate-based gel 308 and the mold 306 is removed, as shown in FIG. 28. The gel 308 is then sterilized and rinsed with, for example, water.

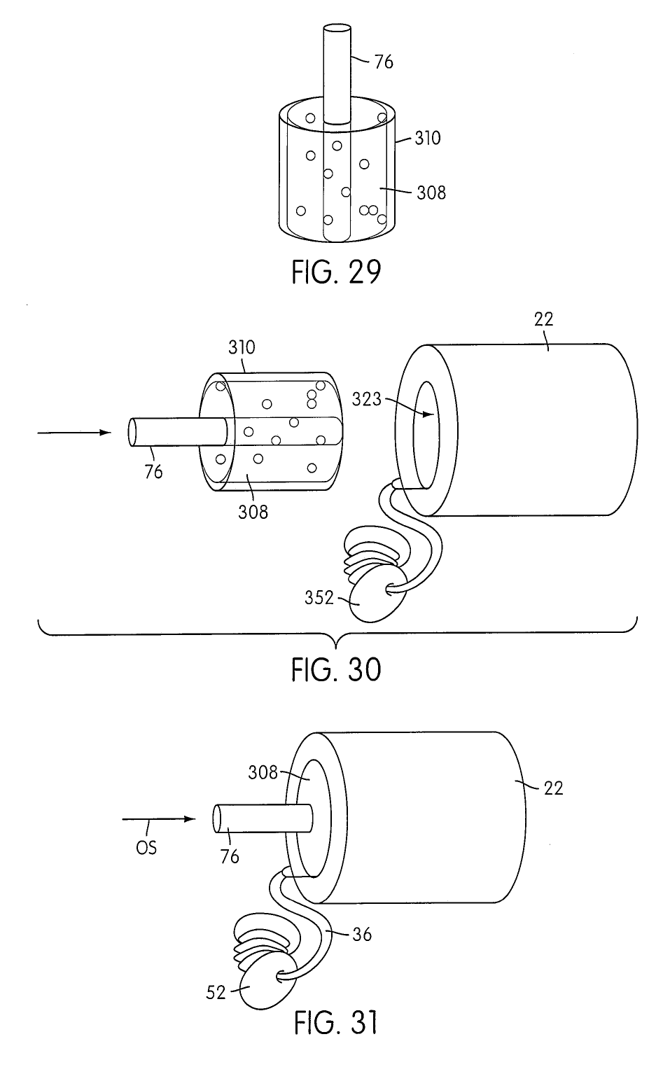

Shown in FIG. 29, the alginate gel 308 is then roll coated in alginate and placed in a barium chloride bath, e.g, for about 15 minutes to allow cross-linking to form a cell barrier 310 around the gel. The coated gel 308 is again sterilized and rinsed.

As shown in FIG. 30, the gel 308 and catheter 76 may then be placed into the vascularized, porous polyurethane container 22 that has been implanted into a patient. A separate pump 52 may be used to deliver immunosuppressants, a growth factor cocktail, or other materials and/or agents to an inner space 323 of the container 22 and/or to a location on an outer surface of the container 22. FIG. 31 shows the device with alginate-based gel 308 and silicone oxygen supplying (OS) catheter 76 placed within the vascularized polyurethane container 22.

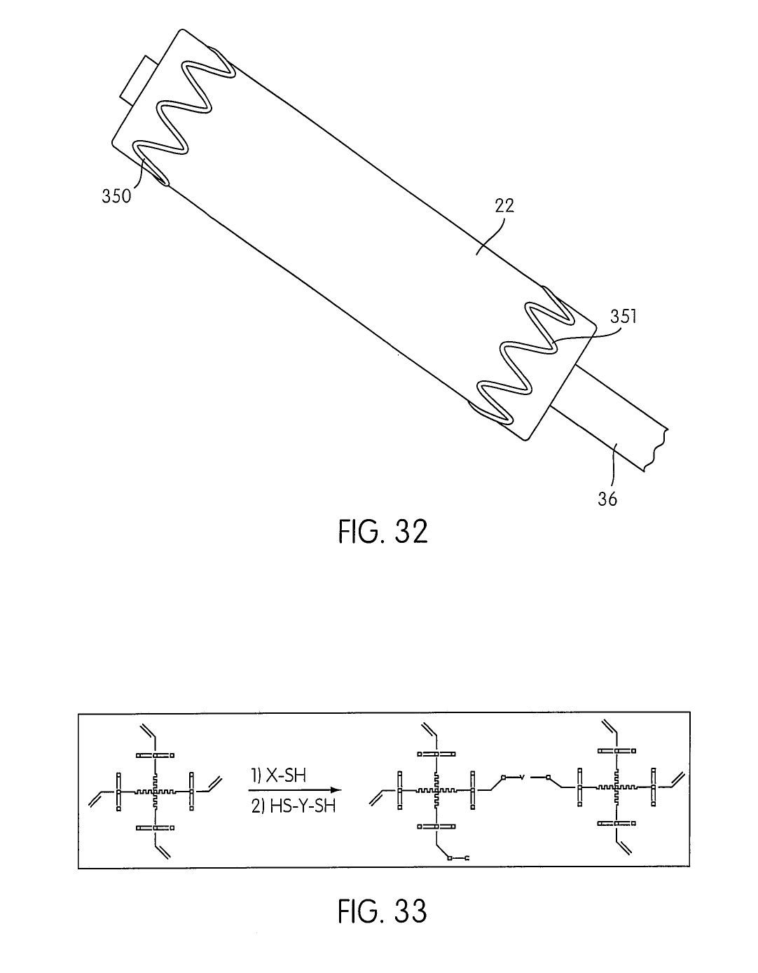

FIG. 32 is a schematic representation of an assembled alginate-based cell therapy device. The device in this embodiment includes wire springs 350, 351 disposed around the cell container 22 in order to retain the container 22 over the hydrogel. It is appreciated that wire springs 350, 351 shown in this embodiment and sutures 50, 51 shown in previous embodiments may be interchangeable.

In a further embodiment, the supply of oxygen to cells contained within an encapsulation device could be enhanced by selecting cell encapsulation matrix having desirable oxygen transport properties, thereby reducing or eliminating certain oxygen transport limitations. The term “cell encapsulation matrix” is understood to refer to material immediately surrounding the therapeutic cells, including, but not limited to, matrix materials described above, e.g., a hydrogel such as alginate, agarose or PEG. Matrix materials may be selected to permit acceleration of the oxygen mass-transfer rate to encapsulated cells or tissue for a given supplied pO₂ level at the boundary of the matrix material, where O₂ may be provided by the vasculature and/or an artificial O₂ supply. One of skill in the art may select matrix materials with enhanced O₂ permeability in order to reduce the maximum pO₂ level to which the cells/tissue are exposed to achieve a desired level of oxygenation. Similarly, selecting matrix materials with enhanced O₂ permeability may reduce or eliminate the formation of an hypoxic core within a collection of therapeutic cells, e.g., a transplanted islet (Islet of Langerhans). In an exemplary embodiment, a matrix formed from an emulsion of perfluorocarbon (PFC) (Oxygent™, Alliance Pharmaceutical Corp., San Diego Calif.) can enhance transport of oxygen to cultured muscle tissue in vitro (Radisic et al., Am J Physiol Heart Circ Physiol 288: H1278-H1289 (2005)). In another non-limiting embodiment, entrapping a PFC emulsion within a hydrogel matrix surrounding islets enhances oxygen transport, thereby improving cellular viability and function. In accordance with the non-limiting embodiments described above, ¹⁹F-NMR can be used to monitor the level of oxygen supplied to encapsulated cells or tissues in vivo, thereby allowing in vivo monitoring to determine the spatial pO₂ profiles within transplanted islets or other tissues (Gross et al., Journal of Theoretical Biology (2006)).

H. Replacement of Therapeutic Cells

In accordance with an aspect of the present invention and referring back to the embodiments shown in FIGS. 6-19, the hydrogel construct 54, 62 encapsulating therapeutic cells may be periodically replaced. The present invention is configured to allow the removal and replacement of the hydrogel construct 54, 62 from and to the vascularized cell container 22 without substantially disrupting the blood vessels formed thereon. Because the blood vessels have not been permitted to form within the interior wall 22a of the container 22, each of the hydrogel constructs 54, 62 herein described may be easily pulled out for replacement. For example, tweezers or other suitable instrument may be used to grip the construct 54 (in the case of, for example, the PEG-based construct) or construct support 64 (in the case of, for example, the agarose-based construct) once the pump 52 and end cap 38 have been removed from the device 10. Upon preparation of a new construct, it may be inserted into the container 22 in a similar manner as previously described for the first construct.

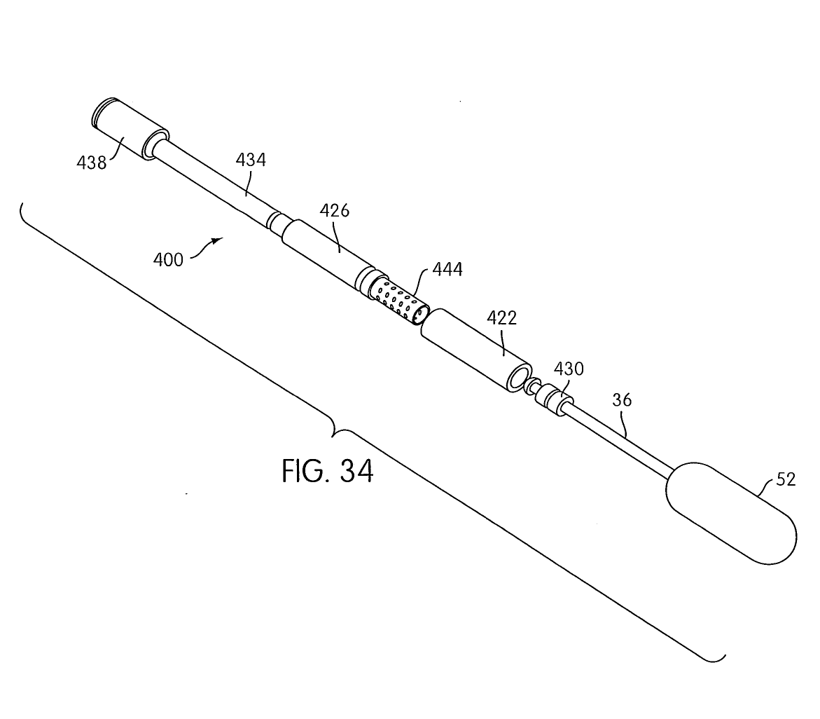

In accordance with another embodiment of the present invention, a vascularization assembly 400 is shown in FIG. 34. As shown in exploded view in FIG. 34, the vascularization assembly 400 includes a pump 52, a tube or catheter 36, a grommet 430, a container 422, a perforated inner tube 444, an outer tube 426, a stopper 434, and an end cap 438. The vascularization assembly 400 operates in a manner similar to that of the vascularization assembly 100 with ePTFE cell barrier 24 described above, except that the pump 52 is at the side of the device 400 opposite the side where the cell construct is inserted and/or removed.

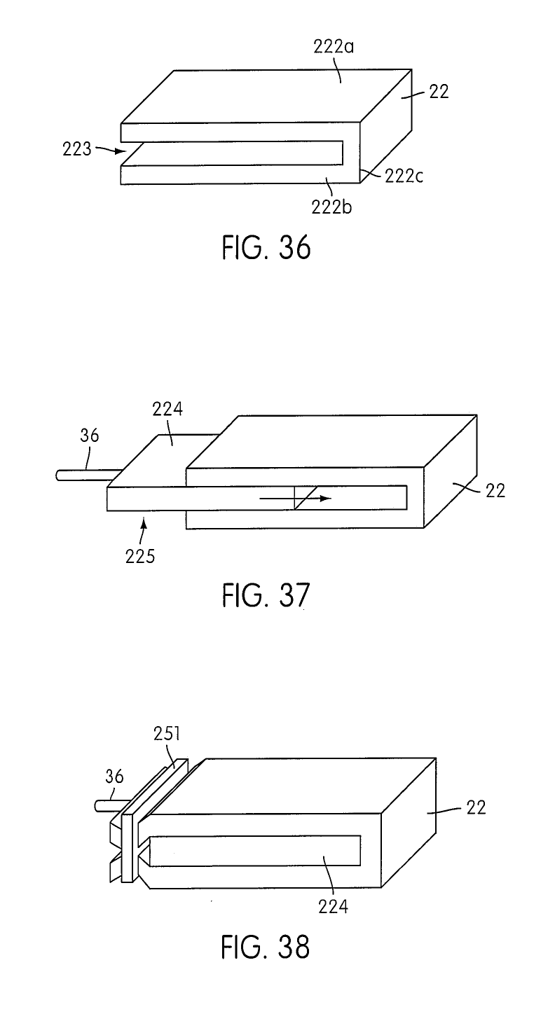

In accordance with a flat sheet or pouch type cell therapy device, such as the device described above with respect to FIG. 3 and FIG. 4, FIGS. 36-51C are schematic views illustrating various steps in the manufacture of such a device 200. In each of FIGS. 36-49, a proximate sidewall is not shown, in order to reveal the interior cavity in container 22.

FIG. 36 is a schematic view of a flat sheet cell container 22 in accordance with one embodiment (the proximal sidewall having been removed in this Figure to show the interior of the container 22). The container 22 may be constructed of similar materials as described above, such as porous polyurethane. The container 22 in this embodiment is generally rectangularly shaped (as can be appreciated from the illustration in FIG. 3), and has two relatively large flat sheets 222a, 222b connected at each of their sides by relatively shorter sides of the container (only one side 222c is visible in FIGS. 36-48). The container 22 forms a flat sheet space or lumen 223 between the sheets 222a, 222b, and the surrounding sides.

As shown in the schematic view of FIG. 37, a cell barrier assembly 225 may be inserted into the lumen 223 in order to vascularize the container 22. The cell barrier assembly 225 is similar to the cell barrier assembly 125 described above except that it has a generally rectangular flat shape. That is, the cell barrier assembly 225 may include a cell barrier 224 and a barrier support (not shown) within the cell barrier 224 in order to give structure to the cell barrier 224. A feed tube 236 may be provided in order to supply various agents or materials as described above (e.g., vascular growth factors). Alternatively, a solid permeable block structure (such as permeable ePTFE material or sintered material such as steel), as in the case of FIG. 4, the disclosure of which is incorporated here in this embodiment. It should be appreciated, therefore, that the container 22 and cell barrier assembly 225 may have a trapezoidal configuration, as described above with respect to FIG. 4.

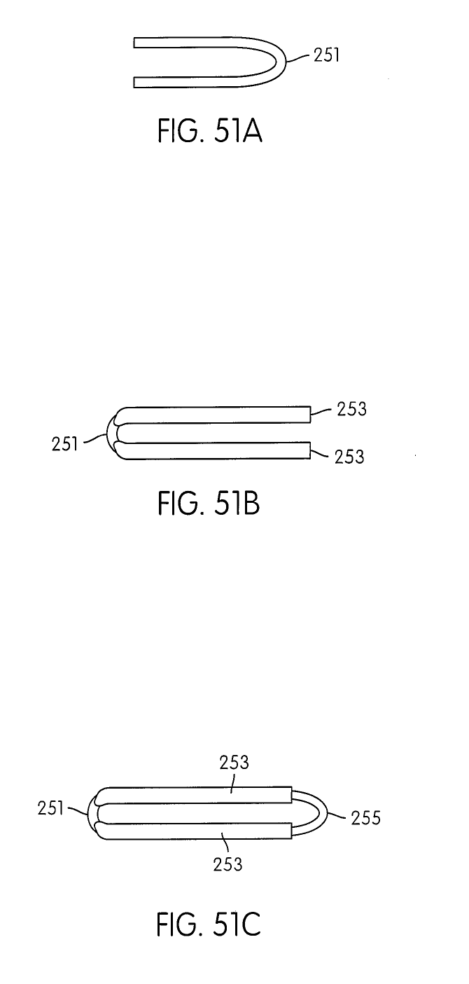

In FIG. 38, a clip 251 is shown sealing the cell container 22 after the cell barrier assembly 225 has been inserted. The clip 251 prevents the cell barrier assembly from moving or shifting during vascularization. The clip 251 may be a wire clip as shown in FIG. 51A or, as shown in FIG. 51B, may include rigid tubular reinforcement elements 253 surrounding the underlying wire 251. The reinforcement elements may be made from metal or plastic. Additionally, as shown in FIG. 51C, the clip 251 may include reinforcement elements 253 and further included a second wire portion or clip 255 for further security. The second wire portion or clip 255 is similar to the first clip 251, and both clips have ends thereof received in the tubular reinforceing elements 253 as shown.

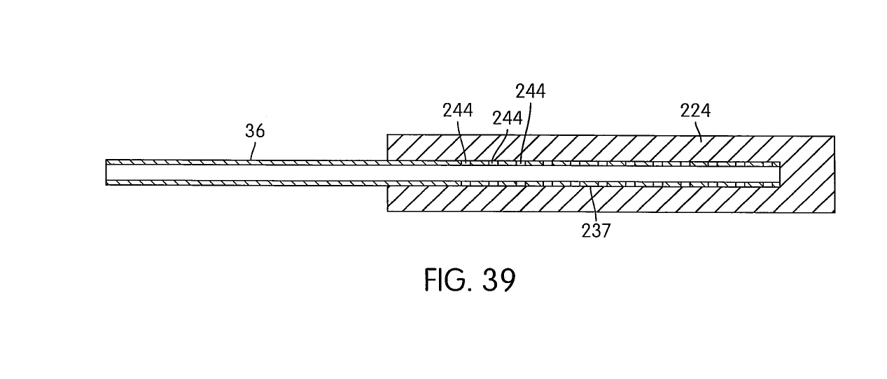

As shown in the schematic cross-sectional view of FIG. 39, the catheter 36 is received in a bore 237 or other opening formed in the cell barrier 224. The catheter 36 in this embodiment may have perforations 244 in order to supply agents to the cell barrier 224 during vascularization. In such a case, a barrier support may be unnecessary. The catheter 36 supplies agents to the cell container 22 via passage through the cell barrier 224 which, as described above, may be suitably permeable to supplied materials and agents but not to blood vessels (e.g., sintered material or ePTFE).

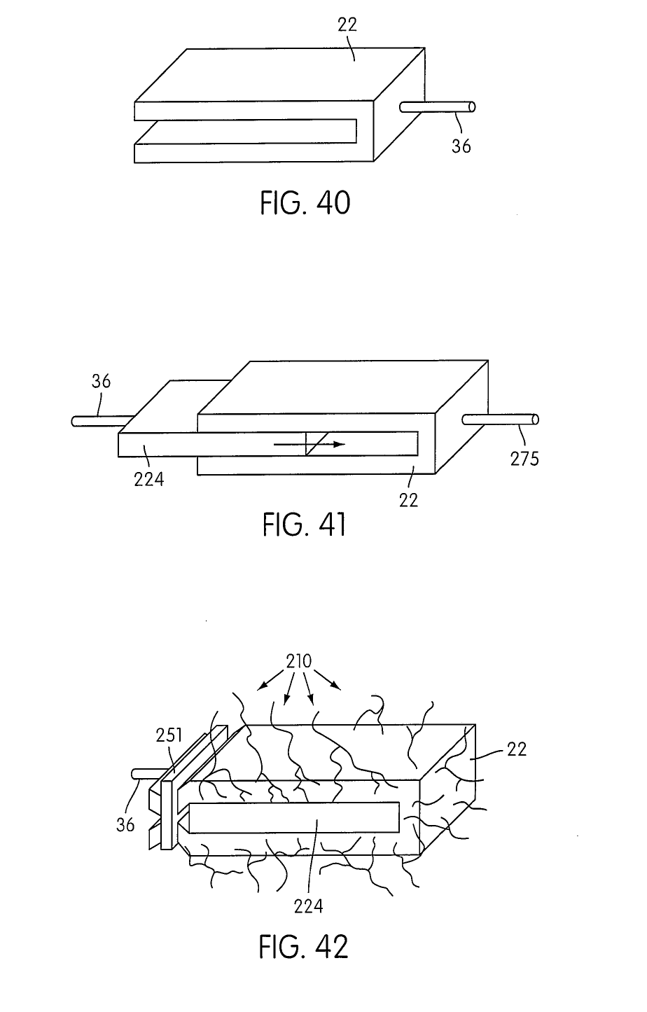

Alternatives to the flat sheet vascularization process just described are depicted in the schematic cross-sectional views of FIGS. 40 and 41. FIG. 40 shows the supply tube 36 on the opposite side of the cell container 22, and FIG. 41 includes a venting tube 275 for avoidance of air pockets or vacuum effects as described previously with respect to the tubular cell container embodiments.

FIG. 42 (again shown with the proximal side wall removed to illustrate the inner cavity of container 22) illustrates the device of FIG. 38 after sufficient vascularization has taken place, including newly formed blood vessels 210. The period of time for this stage to occur may vary and may be determined as described above.

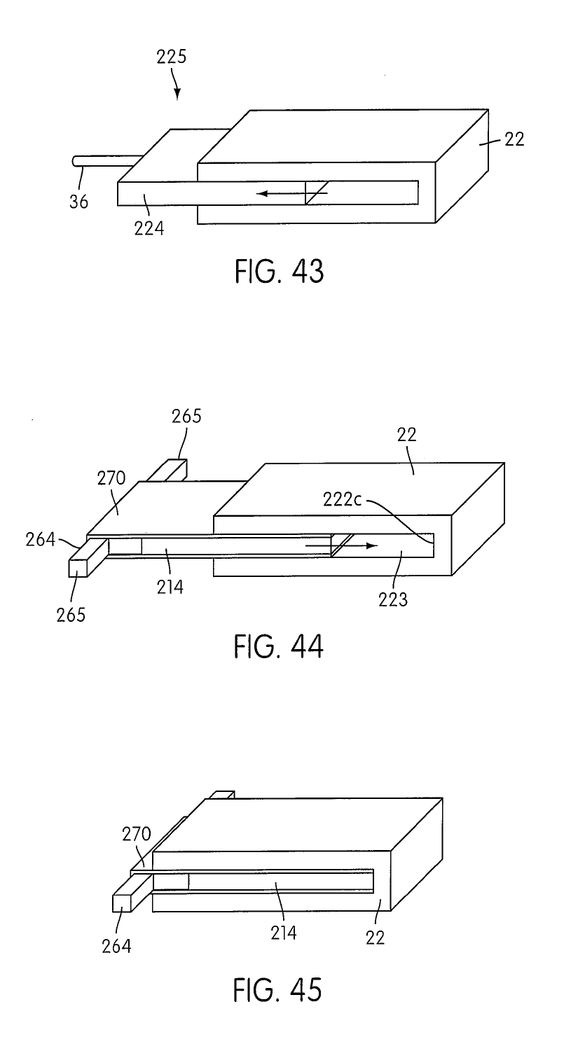

FIG. 43 is a schematic view showing the removal of the cell barrier device 225 after the clip 251 has been removed (not shown). It is noted that FIGS. 43-48 show a vascularized container 22 as in FIG. 42 but, for purposes of clarity, the blood vessels are not shown in Figures.

FIG. 44 is a schematic cross section showing the insertion of a hydrogel cell construct 214, which may be formed as described above and be made of any disclosed or otherwise suitable material. The hydrogel may be delivered into the container by an insertion tool 264 and a U-shaped protective sleeve 270. The protective sleeve 270 serves a purpose similar to the mandrel 56 described in FIG. 12D in that it supports the hydro gel 214 during insertion and prevents over-insertion by abutting distal wall 222c. The protective sleeve 270 also serves a purpose similar to the washers 66, 68 described above in that it protects the hydrogel 214 from damage due to abrasion against the inner surfaces of the container 22 during insertion. Accordingly, the sleeve 270 may be formed of a relatively smooth, rigid material.

The insertion tool 264 may include outwardly extending flanges 265 that may, additionally or alternatively to the protective sleeve 270, protect against over-insertion and subsequent compression and/or damage to the hydrogel. The flanges 265, similar to annular flange 59 in FIG. 12D, may abut against the side walls (not shown) of the container 22 when the hydrogel 214 has reached its maximum insertion distance to prevent any further insertion into the cavity 223. Alternatively, flanges 265 may simply serve as handles for manipulating the tool 264. The insertion tool 264 may also be configured to allow a user to grip the tool and pull it back out from the inner lumen 223 of the container 22. FIG. 45 shows the therapy device after full insertion of the hydrogel 214. An alternative embodiment 266 for the insertion tool is shown in FIG. 50B. The alternative embodiment insertion tool 266 may include legs 267 that may provide additional support to the hydrogel 214 and prevent over-insertion and consequent damage.

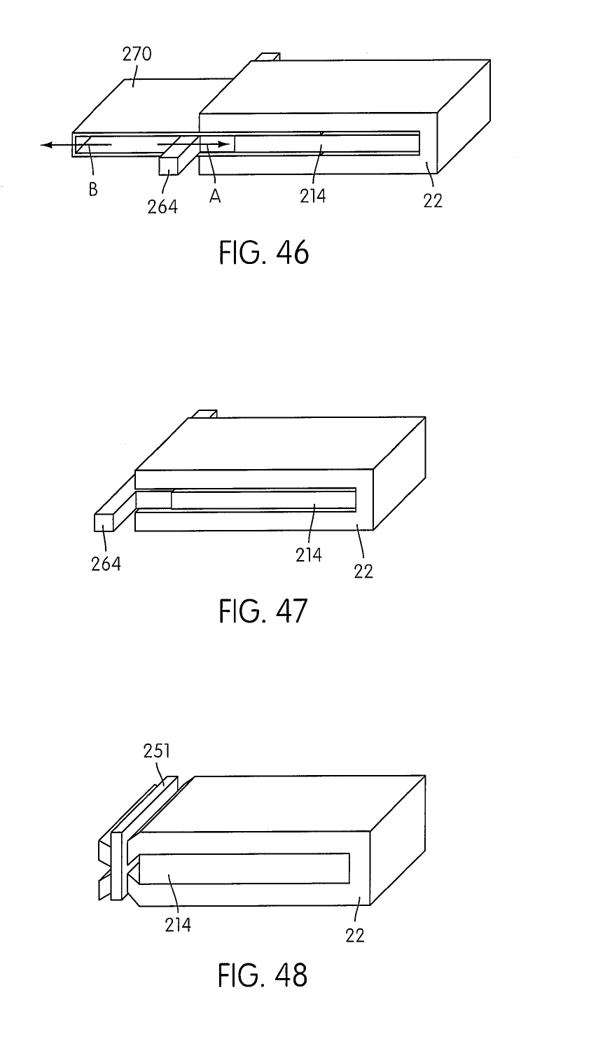

As shown in FIGS. 46 and 47, the insertion tool 264 and protective sleeve 270 are then removed. First, while the insertion tool 264 is held with a force in the direction of arrow A, the protective sleeve 270 may be gripped and pulled from the container in the direction of arrow B. With the removal of the sleeve 270, the hydrogel 214 and encapsulated cells may be in direct or at least unobstructed close proximity to the vasculature 210 formed in the container 22.

With reference to FIG. 47, the insertion tool 264 may then be removed from the container 22 and the container 22 is then sealed with a clip 251, as shown in FIG. 48. The container 22 may alternatively be sealed by a clip from among those shown in FIGS. 51A-C.

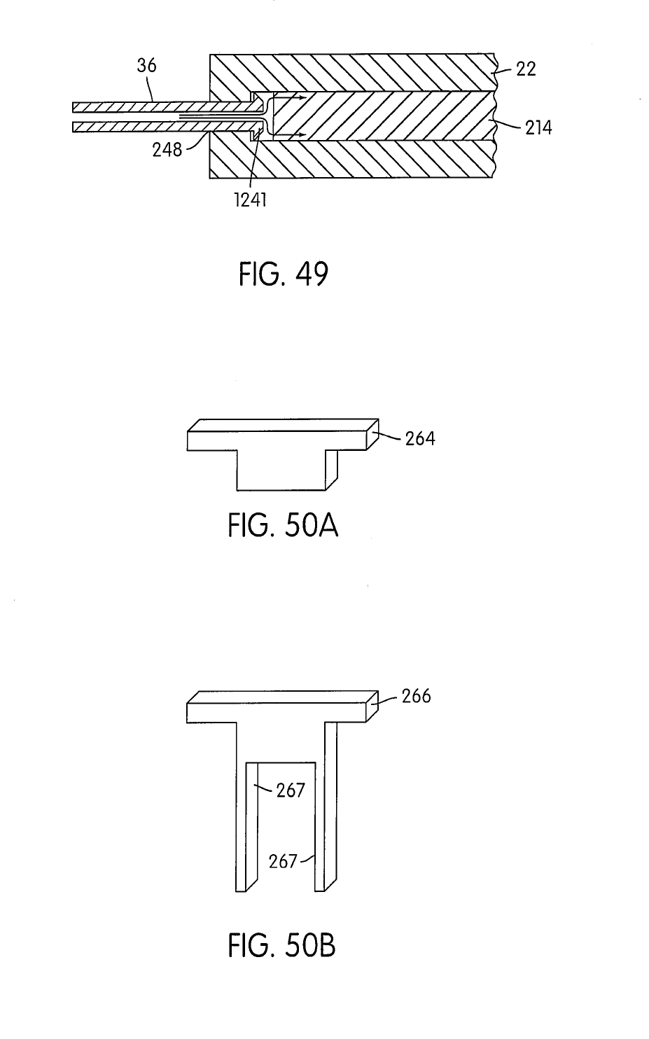

As shown in FIG. 49, a catheter 36 may be connected (or may be connected prior to attachment of clip 251) through bore 248 in container 22 to supply materials or agents as described above with respect to materials supplied to the tubular cell container embodiments (e.g., immunosuppressants, oxygen, growth factors, etc.). In contrast to the tubular container configuration, materials in the present flat container embodiment may be supplied to a space between the hydrogel 214 and the container.

In one embodiment, as illustrated in FIG. 49, the catheter 36 may be connected to the container 22 by a radially outward extending flange 1241 that can be inserted into the narrowed opening or bore 248 leading into the container 22, such that the flange 1241 is received behind the walls defining the narrowed opening 248.

It is appreciated that various configurations of the device can be realized, such that materials and agents as described above may be supplied to encapsulated therapeutic cells by proximal pumping, distal pumping, or proximal and distal pumping. In one embodiment, proximal pumping into a flat pouch device is achieved using a ported PTFE spacer positioned at the proximal end of container 22 and operatively connected to a pump via a catheter or supply tube, e.g., pumping through a catheter 36 positioned as in FIG. 37, where the ported PTFE spacer may be positioned distal to the clip 251 as shown in FIG. 38. In another embodiment, distal pumping into a flat pouch device is achieved using a ported PTFE spacer positioned at the distal end of container 22 and operatively connected to a pump via a catheter or supply tube, e.g., pumping through a catheter (supply tube) 36 positioned as in FIG. 40, where the ported PTFE spacer may be positioned distal to hydrogel after the hydrogel is inserted into container 22. In another embodiment, proximal and distal pumping into a flat pouch device is achieved using two ported PTFE spacers, one positioned proximal to the hydrogel and one positioned distal to the hydrogel after the hydrogel is inserted into container 22, and wherein each ported PTFE spacer is operatively connected to a pump via a catheter or supply tube.

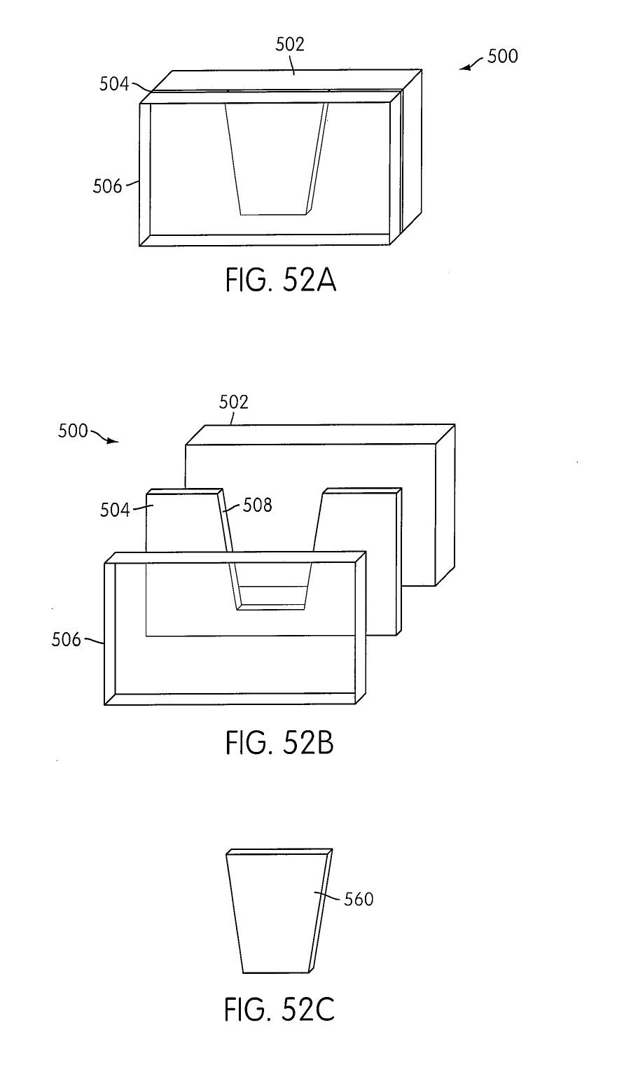

FIG. 52A is a perspective view of a casting mold 500 used to mold a trapezoidal shaped flat sheet hydrogel in accordance with an embodiment of the present invention.

FIG. 52B is an exploded view of the casting mold shown in FIG. 52A. As shown in the embodiment of FIG. 52B, the casting mold has a three-part construction. Specifically, the casting mold 500 is of a three-piece construction, including a first outer portion 502, a middle portion 504, and a second outer portion 506. In this embodiment, each of the portions 502, 504, and 506 are made from an inert material. Such materials may be stainless steel, glass, PTFE, or DELRIN, as non-limiting examples. Each of the mold portions may be formed from the same material, or may be formed from different materials. For example, in the embodiment shown, the first outer portion 502 is made from DELRIN, the middle portion 504 is made from PTFE, and the second outer portion 506 is made from clear glass.

As shown, the middle mold portion 504 has a trapezoidal shaped cutout region 508. When the three mold portions 502, 504, and 506 are secured together (e.g., by adhesive bonding or by clamping, for example) the cutout portion 508 in the middle mold portion 504 defines the trapezoidal shaped side edges of the hydrogel material that is to be molded therein. In addition, the first outer portion 502 defines one of the major sides of the hydrogel sheet, while the second outer portion 506 defines the other major surface of the hydrogel to be molded within the cavity defined by the cutout 508. In this embodiment, the glass material of the outer portion 506 allows the manufacturer to view the hydrogel material being formed within the cavity, since the second outer portion 506 is formed from clear glass.

In another embodiment, the mold 500 can be formed from a single, integrally formed material with a trapezoidal cavity drilled, machined, or otherwise formed therein. The single, integrally formed mold structure can again be formed from any suitable material, preferably an inert material.

The molded hydrogel is illustrated in FIG. 52C.

In one embodiment, the material forming the mold 500 is formed from fully sterelizeable parts to enable continued reuse of the same mold over and again.

In one embodiment, the hydrogel is formed so that it does not substantially expand after it is inserted into a trapezoidal container. This can be done either by making the hydrogel of a material that is fully hydrated prior to insertion into the container, or by pre-swelling an under-hydrated gel prior to insertion in the trapezoidal container. By providing a fully swollen hydrogel prior to insertion, there is less possibility that the hydrogel will buckle or have an undulated or curved configuration resulting from swelling in an uneven manner after being installed in the container.

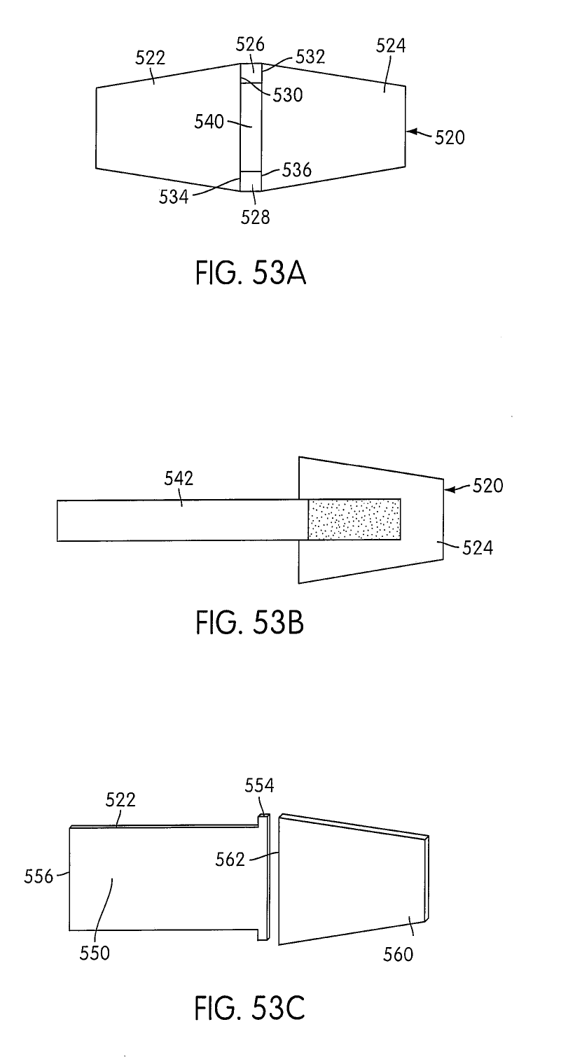

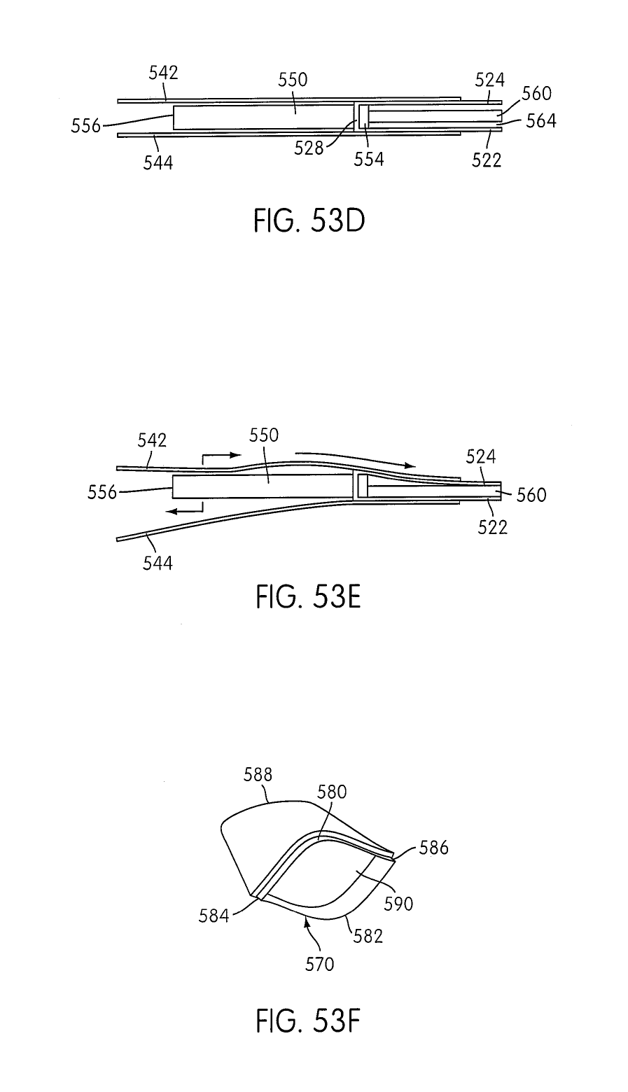

Shown in FIGS. 53A-53F are various steps used for inserting a trapezoidal flat sheet hydrogel into a trapezoidal shaped porous container of the type described above.

FIG. 53A is a plan view of the initial formation of a shovel head 520 used to insert a trapezoidal hydrogel 560 (as seen in FIG. 52C) into a container (as seen in FIG. 53F). The hydrogel can be made in accordance with any of the teachings herein. The shovel head 520 as shown has been stamped or otherwise formed from a Mylar sheet material. In one embodiment, the Mylar sheet is 500ₘₘ thick. The shovel head has two flat trapezoidal sections, 522 and 524. The trapezoidal sections have a peripheral configuration that is substantially of the same dimension as the trapezoidal hydrogel to be inserted into a trapezoidal container. The two trapezoidal sections 522 and 524 are joined at the larger of their associated parallel sides by a pair of spaced hinges 526 and 528. Each of the hinges defines a pair of spaced fold lines (530 and 532 for hinge 526 and fold lines 534 and 536 for hinge 528). In addition, between the hinges 526 and 528 is an opening 540 as seen in FIG. 53A.

The first trapezoidal section 522 is folded with respect to the hinges 526 and 528 along the associated fold lines 530 and 534, while the second trapezoidal section 524 is folded with respect to hinge portions 526 and 528 along the associated fold lines 532 and 536. The resulting folded configuration of the shovel head 520 is illustrated in FIG. 53B. In addition, after the shovel head is folded, a pair of handles 542 and 544 are glued to the exterior surface of the associated trapezoidal sections 524 and 522, as can be appreciated from FIGS. 53B-53E.

Next, as illustrated in FIG. 53C, a pusher member or plunger 550 is inserted into the shovel. Specifically, as shown in FIG. 53C, the plunger 550 has a generally flat, rectangular body portion 552, and an enlarged forward flange portion 554. As can be appreciated from FIG. 53B, the enlarged portion 554 of the plunger 550 can be manually grasped such that the trailing end 556 of the plunger 550 is inserted between the shovel head sections 522, 524 so that it is received through opening 540 until the flange portions 554 engage the hinges 526 and 528 (i.e., the plunger 550 is moved toward the left in FIG. 53D until the flange portion 554 engages the hinges 526, 528).

After the plunger has been inserted into the shovel, the trapezoidal hydrogel 560 may be scooped by the shovel head 520 by manipulation of the handles 542, 544 so that the larger of the two parallel edges 562 of the trapezoidal hydrogel is received first within the cavity 564 defined between the first and second trapezoidal sections 522, 524 of the shovel head 520.

After the hydrogel 560 is received within the trapezoidal cavity 564, the shovel head 520 can be used to insert the hydrogel 560 into a trapezoidal container 22 as illustrated in FIG. 53F.

In one embodiment, the shovel handles 542, 544 may be manipulated in opposite directions so that the trapezoidal portions 522, 524 of the shovel head 520 apply a gentle gripping force on the hydrogel 560, as illustrated in FIG. 53E. Specifically, the handle portion 542 may be pushed slightly towards the shovel head 520, while the lower handle portion 544 is gently pulled away from the shovel head 520, and the resulting shovel head portions 522, 524 will flex inwardly to grip the trapezoidal hydrogel 560 as illustrated in FIG. 53E.

In the embodiment illustrated in FIG. 53F, the porous container 22 is of a trapezoidal shape, having an opening formed at the larger of the two parallel sides thereof. The container has normally flat configuration formed by flat sides 580 and 582 thereof, which are connected along the side edges 584 and 586 thereof, as well as along the bottom edge 588 thereof.

In the illustration of FIG. 53F, an individual is shown pinching the side edges 584 and 586 toward one another so as to enlarge the opening 590 formed between the larger parallel edges of the trapezoidal sides of the container 22. This action is much the same as the type of action used in many conventional womens' purses.

In the configuration shown in FIG. 53F, the container 22 is ready to receive the flat hydrogel. Grabbing and manipulating the handles 542 and 544, with the plunger 550 pinched therebetween, the shovel head 520 is inserted into the opening 590 of the container 22. After the hyrdrogel 560 is fully inserted into the generally correspondingly shaped opening within the container 22, the shovel head 520 is pulled outwardly from the confines of the container 22, while the rear end 556 of the plunger 550 is engaged and moved relative to the shovel head 520 so as to ensure that the hydrogel is retained within the container 22 as the shovel head 520 is withdrawn. Thus, it can be appreciated that while the shovel head is withdrawn, the container 22, the hydrogel 560 and the plunger 550 all remain relatively stationary, while the shovel head 520 is moved, thus leaving the hydrogel 560 within the container 22.

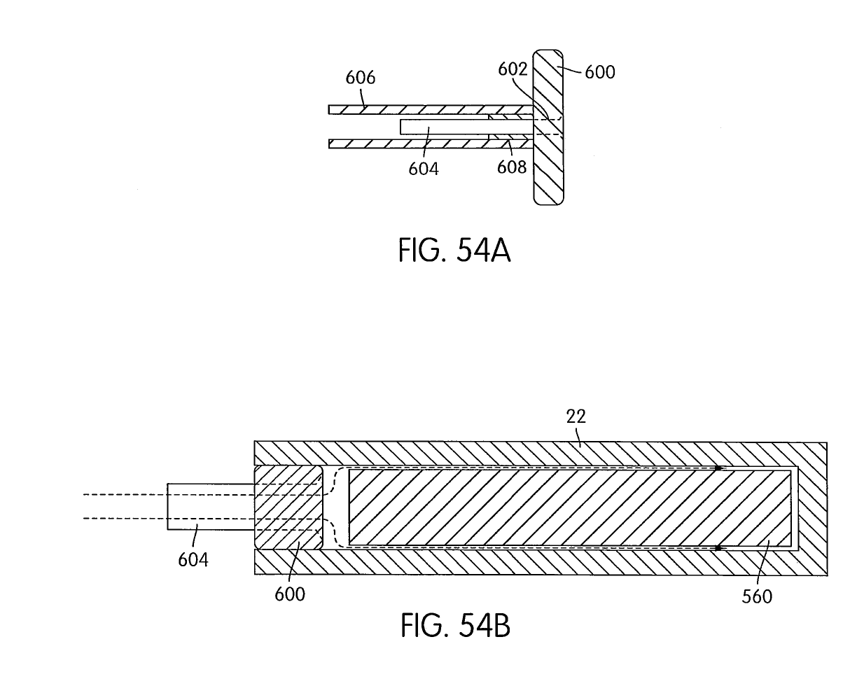

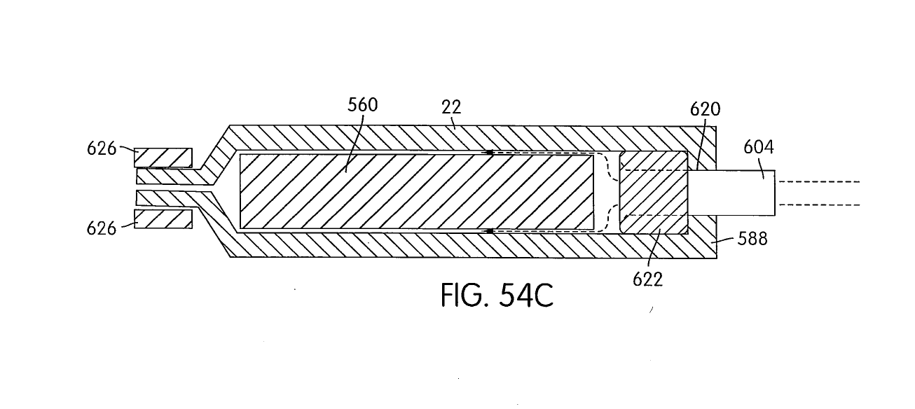

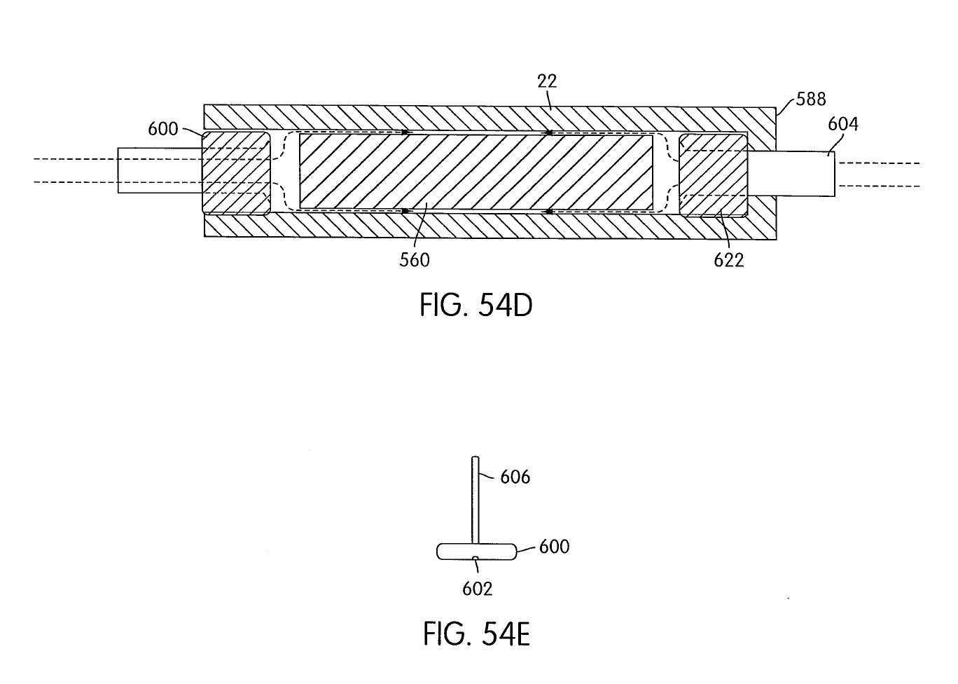

FIG. 54A-54E illustrate various mechanisms for plugging the trapezoidal shaped container 22. Specifically, FIG. 54A is a cross-sectional view of a plug member 600 used to seal the opening 590 in the porous container 22. The plug 600 can be made from an inert material, such as PTFE. The plug 600 has a central bore 602 therethrough for receiving a catheter 604. The catheter 604 should be made from an inert material, such as stainless steel. The catheter 604 is shown surrounded by another catheter or pipe 606. In the illustrated embodiment, the catheter 606 is formed from a polyethelyne material. Between the catheter 604 and catheter 606 is disposed a spacer 608 that is squeezed between the two catheters 604, 606 so as to retain the outer catheter 606 in place. The outer catheter 606 is adapted to receive agents to be pumped or otherwise provided to the hydrogel contained within the container 22.

In some embodiments, catheters 604 and/or 606 can optionally be made in accordance with any of the teachings with respect to catheters 36 discussed above.

FIG. 54B illustrates the hydrogel 560 within the trapezoidal container 22. The plug 600 is shown closing off the open end of the container 22. The plug 600 in FIG. 54B is shown rotated by 90° in comparison with the view illustrated in FIG. 54A. This can be more fully appreciated from the perspective view of FIG. 54E, illustrating the plug 600 and catheter 606.

FIG. 54C illustrates an alternative pumping arrangement. In this arrangement, the catheter 604 is inserted into the container 22 first and is used to pierce a small hole 620 in the smaller parallel connected edge 588 of the container 22. In this arrangement, a stop structure 622 at the end of catheter 620 prevents the catheter from being pulled all the way through the container. In this embodiment, the hydrogel 560 is inserted into the container after the stop 622 and catheter 604 are put in place. In this embodiment, the larger opening that receives the hydrogel 560 is pinched closed by a clamp structure 626.

In the embodiment of FIG. 54C, the hydrogel 560 can be removed by removing the clamp 626, but leaving the catheter 604 in place for use with the next hydrogel 560 placed into the container 22.

The embodiment of FIG. 54D illustrates a combination of FIGS. 54B and 54C. Specifically, FIG. 54D illustrates an embodiment wherein pumping is performed from opposite sides of the container 22. Specifically, the same catheter 604 and plug 622 is inserted first into the container 22 so that the catheter 604 pierces an opening 620 in the wall 588 of the container (it should be noted that in this embodiment, as well as in the embodiment of 54C, the catheter 604 is subsequently connected to an outer catheter or pipe 606 as illustrated in 54A). After the catheter 604 and stop structure 622 are inserted into the container 22, the hydrogel 560 is placed into the container 22, and then the large opening that receives the hydrogel is closed off by the plug 600 and catheter 604, as illustrated in the embodiment of 54B.

In the embodiment of FIG. 54D, different agents, or the same agent, can be pumped or otherwise provided into the container 22 from opposite sides thereof. In one embodiment, liquid agent is pumped into one side, while gaseous agent (e.g., oxygen) is pumped into the other side. In the embodiments of FIGS. 54A-54D, the agents can be received by the hydrogel 560 along the exterior surface areas of the hydrogel 560, including between the interface of hydrogel 560 and the container 22, as the agents migrate between the surfaces of the hydrogel 560 and the container 22.

### EXAMPLES

### Example 1

### Preparation and Evaluation of APGS

A. Preparation of APGS

Whole blood was drawn from 4 human donors (2 male and 2 female) to give a starting sample of sixty (60) ml of whole blood for each donor, which was then used to obtain generate preparations of normal serum and APGS. The blood was mixed with sufficient sodium citrate to act as an anticoagulant. Sixty (60 ml) of blood from each donor was processed using a Medtronic Magellan™ Autologous Platelet Separator (Medtronic Inc., Minneapolis Minn.) to obtain 6 ml of platelet rich plasma (PRP) from each donor. Approximately 6000 units of thrombin (at 1000 U/ml) was added to the PRP and mixed by repeated pipetting. The thrombin/PRP mixture was allowed to clot for about 15 minutes at room temperature, to generate autologous platelet gel (APG, clotted PRP). The APG was then centrifuged at 13,000 rpm for 5 minutes. The supernatant was removed and saved as autologous platelet gel serum (APGS). A total volume of approximately 6 ml of APGS was obtained.

Samples of normal serum and APGS were sent to Pierce Biotechnology, Inc. (Rockford, Ill.) for enzyme-linked immunosorbent assay (ELISA) analysis to determine the levels of 16 compounds related to angiogenesis (angiogenic factors): VEGF, bFGF, PDGF-BB, PLGF, BDNF, TNFα, TGFα and (3, GMCSF, HB-EGF, HGF, KGF, Ang2, TPO, and TIMP 1 & 2).

B. In Vitro Stability of APGS

The stability of APGS components was measured under in vitro conditions designed to mimic the in vivo conditions following implantation. Two hundred microliters (200 μl) each of APGS and normal serum were incubated in an Alzet® osmotic pump for 14 days at 37° C. After 14 days, each sample was analyzed to determine levels of VEGF, bFGF, PDGF-BB, PLGF, BDNF, TNFα, TGFα and β, GMCSF, HB-EGF, HGF, KGF, Ang2, TPO, and TIMP 1 & 2 using ELISA as described above, and compared with the levels of each angiogenic factor in each sample measured prior to incubation. Ang2 was the only growth factor that showed a substantial decrease in measurable levels: in normal serum, Ang2 was nearly non-detectable and in APGS, levels of Ang2 dropped from approximately 600 pg/ml to approximately 100-150 pg/ml after 14 days incubation at 37° C. Based on these results, it was concluded that APGS contains several angiogenic growth factors that will be stable in vivo.

C. Effects of APGS on Endothelial Cells In Vitro

Endothelial cell proliferation. The effect of APGS on the proliferation of endothelial cells in vitro was studied as an indicator of the effectiveness of APGS to simulate angiogenesis. Human coronary artery endothelial cells (HCAEC) in endothelial growth medium (EGM-2) with 5% fetal bovine serum (FBS) were plated in wells of a 96-well culture plate at 3,000 cells/well, and then serum starved for 24 hours. Cells were then grown for 72 hours in one of the following culture conditions: EGM-2; endothelial basal medium (EBM); EBM with 25 ng/ml VEGF, EBM with 10% normal serum (NS); EBM with 10% APGS, or EBM with 20% APGS. Each culture condition was carried out in triplicate (n=3). Cell viability was assayed using the CellTiter 96® Aqueous One Solution Cell Proliferation Assay (Promega Corp., Madison Wis.). Results were calculated as the mean+/−SD of triplicate experiments, and P values of certain pairwise comparisons were calculated.

Endothelial cell proliferation was significantly greater in wells containing APGS than in wells containing normal serum (P<0.01 for 20% APGS, P<0.05 for 10% APGS). As expected, endothelial cell proliferation in wells containing EBM alone was even lower than proliferation in wells containing normal serum. Endothelial cell proliferation in wells containing APGS was similar to that in wells containing VEGF, suggesting that APGS and VEGF treatments were very similar in their ability to stimulate cell proliferation.

Endothelial cell migration. The effect of APGS on endothelial cell migration in vitro was studied as another indicator of the effectiveness of APGS to simulate angiogenesis. HCAEC cells were fluorescently labelled and plated on inserts placed in the culture wells (BD insert, BD Biosciences, San Jose, Calif.) and allowed to migrate through pores in the insert toward the same culture conditions as described above, here presented as chemoattractants: EGM-2 (“growth media”); EBM (“basal media”); EBM with 25 ng/ml VEGF, EBM with 10% normal serum (NS); EBM with 10% APGS, or EBM with 20% APGS. The concentration of fluorescently labelled cells in the culture medium below the insert was measured when the cells were plated on the inset (t=0) and again after 4 hours. More HCAEC cells migrated towards solutions containing APGS, VEGF, or growth media than towards normal serum or basal media. Thus, APGS appeared to be similar to VEGF in its ability to stimulate endothelial cell migration.

D. APGS Effects on Angiogenesis In Vivo

The effect of APGS delivery to tissues surrounding a subcutaneous implant (graft) was measured to evaluate the effectiveness of APGS to promote angiogenesis in vivo. Four (4) groups of male nude rats were used, with 3 rats (n=3) treated with each test solution. Each animal was subcutaneously implanted at the back of the neck with a cylindrical container (or implant, also called “graft” following implantation) having a porous outer layer of polyurethane (PU) that is 350 μm thick, with pores 150-180 μm in diameter, and having an inner cell barrier of low porosity ePTFE (expanded polytetrafluoroethylene) that is 350 μm thick, and a hollow interior. The implanted container (graft) was connected to an Alzet® osmotic pump (Model 1002, internal volume approximately 200 μl) by means of a blunt metal tube protruding from the pump, and the polyethylene catheter protruding from the proximal end of the graft, such that the catheter fit over the metal tube to form a sealed channel. Treatment solutions were introduced into the pump using a 21 gauge needle.

For each rat, one of the following treatments was delivered by the osmotic pump into the hollow interior of the graft and through the ePTFE cell barrier and the PU outer layer into the surrounding tissues: PBS buffer control (150 mM NaCl, 150 mM NaPO₄, pH 7.2); VEGF (total dose 1 ug/day, lyophilized VEGF resuspended in sterile water), normal serum (NS; obtained from human donors as described in Example 1A above); or APGS (approximately 200 μl per day, obtained from human donors as described in Example 1A above). After 10 days of treatment, animals were euthanized, the container was photographed while in the animal, and then explanted for histological evaluation. The PU container was cut in half longitudinally and fixed in zinc, then processed, and finally embedded in paraffin. Slides were prepared from 3 μm longitudinal cross sections of the graft and tissue, and the zinc-fixed tissue was stained using anti-rat-CD31 antibody and DAKO LSAB 2 Alkaline Phosphatase kit (DAKO Cytomation, Carpinteria Calif.) to specifically stain endothelial cells. Anti-CD31 stained sections were then photographed with a CoolScope (Nikon Corp., Japan) and the images were processed with a software package VIS (Visiopharm, Hørsholm, Denmark).

Histological analysis (visual evaluation) of the anti-CD31-stained sections indicated that both VEGF and APGS created a more highly vascularized network compared to PBS and normal serum. The vessels seen in the stained sections appeared similar to the vessels commonly known as feeder vessels, which may branch into smaller vessels and subsequently, capillaries.

With the VIS software package, it was possible to digitally analyze the stained sections (sections of porous container and surrounding tissue) to quantify the amount and configuration of anti-CD31-stained vessels and thereby to determine parameters such as vessel diameter and morphology, and the total area occupied by vessels. Total vessel area was expressed as the percentage of the total area of the outer surface of the porous container (% total PU area) occupied by blood vessels. By measuring the blood vessels in the outer surface layer of the container, it was possible to determine how much blood vessel formation had occurred in the container—i.e., it was possible to provide a quantitative measure of angiogenesis, or vascularization of the implanted container. When the total area occupied by blood vessels in the outer surface layer of the porous polyurethane container was compared, it was determined that exposure to APGS resulted in a significantly greater total area of vessels (P<0.1) than the number of vessels formed after exposure to normal serum (NS). Exposure to NS and PBS buffer control resulted in similarly low total vessel area measurements. Exposure to VEGF resulted in the highest total area of vessels, and exposure to APGS produced similar results. These results indicate that APGS creates more vasculature in vivo throughout a porous container, i.e., induces more vascularization of an implanted device, than normal serum, and APGS induces levels of vascularization similar to VEGF.

E. Effects of GF Mixtures and APGS on Angiogenesis In Vivo

The in vivo angiogenic effects of delivering mixtures of growth factors (GF mixtures) and neutralizing anti-growth factor antibodies to tissues surrounding a subcutaneously implanted container in accordance with the invention were measured. Five (5) groups of male Lewis rats were used for the study, with three rats (n=3) treated with each test solution. Each animal was subcutaneously implanted at the back of the neck with a construct as described in Example 1C above, and the implanted device was connected to an Alzet® osmotic pump (Model 1002) as described in Example 1C above.

For each rat, one of the following treatments was delivered by the osmotic pump into the hollow interior of the container and through the ePTFE cell barrier, through the material of the porous container itself, and into the surrounding tissues: PBS buffer control (150 mM NaCl, 150 mM NaPO₄, pH 7.2); VEGF (1 μg/day, in sterile water); VEGF+b-FGF (each growth factor at 500 ng/day, for a total growth factor dose of 1 μg/day); rat APGS (approximately 200 μl); and rat APGS+anti-TIMP1 blocking antibody (total volume approximately 200 μl; it was previously determined that approximately 1 μg anti-TIMP1 antibody was sufficient to neutralize the protein).

Some animals were euthanized after 10 days of treatment (i.e., 10 days of pumping growth factors through the implanted container and into surrounding tissues) and some animals were euthanized after 40 days of treatment (40 days of pumping). For each animal, the container was photographed while in animal, and then explanted for histological evaluation. As described in Example 1C above, the container with its surrounding tissues was cut in half, fixed with zinc, processed, and embedded in paraffin. Slides were prepared from 3 μm longitudinal sections of the container embedded with vessels, and surrounding tissues stained for endothelial cells using anti-rat-CD31 antibody and the DAKO LSAB 2 Alkaline Phosphatase kit (DAKO Cytomation). As in Example 1.D. above, anti-CD31-stained sections were photographed with a CoolScope (Nikon Corp.) and images were processed with the software package VIS (Visiopharm).

For animals that received 10 days of treatment, the total vessel area in animals treated with APGS alone was greater than the total vessel area in animals treated with the PBS buffer control. Treatment with a mixture of APGS+anti-TIMP1 resulted in a total vessel area greater than the total vessel area in animals treated with the PBS buffer control, and slightly lower than the total vessel area in animals treated with APGS alone. Both the VEGF and VEGF+b-FGF (the “GF mixture”) resulted in significantly greater total vessel areas compared with the total vessel area in animals treated with the PBS buffer control. Treatment with APGS or APGS+anti-TIMP1 resulted in total vessel areas that were somewhat lower than the total vessel areas after VEGF or VEGF+b-FGF treatment, but the APGS and APGS+anti-TIMP1 results were nonetheless higher than the PBS buffer control.

For animals that received 40 days of treatment, the total vessel area in animals treated with the PBS buffer control was greater than after 10 days, indicating that some vascularization of the porous container may occur in the absence of added growth factors. After 40 days of treatment, the total vessel area was similar for all animals receiving growth factors, i.e., the total vessel area was similar for animals treated with VEGF alone, a mixture of VEGF+b-FGF, APGS alone, and a mixture of APGS+anti-TIMP1, and all of these values were greater than the total vessel area in animals treated with the PBS buffer control. The total vessel areas in animals treated for 40 days with growth factors were slightly lower, indicating that peak effectiveness of APGS, VEGF, or other angiogenic factors to induce angiogenesis may be reached by about 10 days of treatment (pumping). Finally, a comparison of the results of treatment with APGS and APGS+anti-TIMP1 indicates that neutralization of TIMP1 in APGS does not neutralize the angiogenic effectiveness of APGS.

F. Effects of APGS on Vascularization Measured by MicroCT

The effect of APGS on vascularization of tissues surrounding a subcutaneous implant (graft) was measured using micro-computer tomography (microCT) to determine blood vessel volume. MicroFil® (Flow-Tech Inc., Carver Mass.) a low viscosity, radiopaque silicone liquid that contains the contrast agent lead chromate, was used to perfuse the vasculature for microCT imaging.

Similar to the method described above, male Wistar rats weighing between 250-300 g were implanted with a cylindrical container attached to an Alzet® osmotic pump by a catheter, where the container (also called implant or “graft” following implantation) had a porous PU scaffold 350 μm thick, with pores 150-180 μm in diameter, and an inner cell barrier, also 350 μm thick, of low porosity ePTFE (15 μm), and a hollow interior. Rats were housed at the University of Minnesota in a temperature controlled room (22° C.) following the University's IACUC animal usage protocols.

Pumping. Pumps were filled under sterile conditions with (n=6 for each treatment group) with one of the following solutions: PBS (control); recombinant human Vascular Endothelial Growth Factor (rhVEGF, 1 μg/day); VEGF+bFGF (500 ng each growth factor/day); rat APGS; and rat APGS+anti-TIMP1 blocking antibody. For animals exposed to 10 days of pumping, liquid was released to the (implanted) polymer scaffold by pumping at a flow rate of 0.25 μl/hr. For animals exposed to 40 days of pumping, liquid was released to the (implanted) polymer scaffold by pumping at a flow rate of 0.25 μl/hr.

Perfusion of Vasculature with MicroFil® Imaging Compound.

Rats were anesthetized with an IM injection of Ketamine (90 mg/kg) and Xylazine (10 mg/kg). The dorsal flank site was shaved and scrubbed with Betadine and sterile drapes covered the animal. A 1 cm. incision was made approximately 1 cm. lateral to the spine. The subcutaneous tissue was dissected to form a pocket under the skin. The graft/pump assembly was implanted in the pocket as described elsewhere and the skin closed by placing several single stitches using 4-0 prolene. The animals were allowed to recover under supervision.

After 14 days. the animals were prepared for perfusion. Approximately 30 minutes before perfusion animal were injected with 1000 Units of Heparin IM in the hind flank. Animals were then euthanized using CO₂ fixation. An incision was made to expose the descending aorta, the fascia was removed until the descending aorta was exposed. The aorta was then tied off with suture near the lower branching point of the aorta with another suture wrapped loosely around the aorta ˜1 cm above the previous suture. A small incision was made just enough to nick the aorta about. Using a vein pick catheter introducer (Instech Solomon) PE100 polyethylene tubing was inserted into the aorta. The second suture around the aorta was tied off around the catheter. The vena cava directly under the descending aorta was severed. The PE tubing was connected to a 60 ml syringe using blunt ended 20 gauge needles. Appropriate connectors were used to measure the pressure of the infusion with a pressure transducer (Recorder 2000; Gould Inc., Instruments System Division).

Using a protocol designed for the perfusion of a rat in the range of 150-300 grams in weight, the circulatory system was then flushed system with approximately 60 ml of 0.9% saline with 10 units/ml of Heparin attached to a Harvard Apparatus syringe infusion pump at a rate of 7 ml/min with a pressure of 100 mm HG. During this time the MicroFil® solution was mixed as follows: 4 ml of MV-122 Yellow Compound and 5 ml of MV diluent were mixed, then 450 μl of MV Curing Reagent was added. (The working time of MicroFil® is approximately 20 min, as the three-component mixture will solidify in the vascular system within 15-30 minutes.) The MicroFil® solution was drawn into a 10 ml syringe. The syringe was placed in the infusion pump and pump was elevated so that air bubbles would not go into the circulatory system. MicroFil® was pumped at a rate of 0.5-0.55 ml/min without exceeding 100 mm HG. When the MicroFil® started to flow out the vena cava and started to solidify slightly, the pump was stopped and the rat was allowed to set aside for 30 minutes before the graft was explanted. The polymer scaffold portion of the implant was explanted from the subcutaneous space and immersed in 10% formalin overnight.

Micro-CT Scanning

Grafts were imaged using a high-resolution (8-36 μm isotropic voxel size) micro-CT imaging system (μCT 40, Scanco Medical, Bassersdorf, Switzerland). In addition to the high resolution capabilities, the system offered a larger specimen size (36 mm diameter, 8 cm specimen length). Even at this size, the resolution was around 20 μm. The scanner was set to a voltage of 55 kVp and a current of 145 μA. Resolution was set to high, which created a 1,024×1,024 pixel image matrix. Noise was removed using a low-pass Gaussian filter (=1.2, support=2). The tomograms were globally thresholded based on X-ray attenuation and used to render binarized 3-D images of the vascular network in the grafts. Histomorphometric analysis based on direct distance transform methods, below, was subsequently applied to the 3-D images to quantify parameters of vascular network morphology and anisotropy. All generated x-ray slices were reconstructed using the software provided with the micro-CT scanner. Vessel volume was computed based on the voxel size and the number of segmented voxels in the 3-D image after application of the binarization threshold.

Results. For animals who received 10 days of pumping with PBS, rhVEGF, VEGF+bFGF, rat APGS, and rat APGS+anti-TIMP1 antibody, the animals who received VEGF+bFGF and rat APGS had the highest average vessel volumes. For those that received 40 days of pumping with PBS, rhVEGF, VEGF+bFGF, rat APGS, and rat APGS+anti-TIMP1 antibody, the animals who received VEGF+bFGF and rat APGS had the highest average vessel volumes, and the average vessel volumes after 40 days were slightly higher than the average after 10 days. These results indicate that exposure to APGS produced vessel volumes similar to those produced by exposure to VEGF+bFGF, where these vessel volumes were significantly greater than those measured in animals exposed to PBS (control).

### Example 2

### Silicone Tubing O₂ Permeability Experiments

The capacity for silicone tubing to supply oxygen (O₂) was measured by determining the O₂ permeability of silicone tubing. Calculated (theoretical) and experimental (actual) flux values were determined, and these values were evaluated in terms of the predicted O₂ requirements of islet cells.

A round-bottom flask was filled to the level of the bottom of the stopper with water (235 ml, i.e., no headspace) and equipped for magnetic stirring. An oxygen sensor was threaded through the stopper and immersed in the water. A sealed silicone catheter (tube) was also threaded into the flask and connected to an external O₂ supply. The silicone cathether had a total length of 10 cm in the flask, a wall thickness of 292 microns, and an internal diameter (ID) of 1.204 mm. The flask was then immersed in a water bath at 37° C.

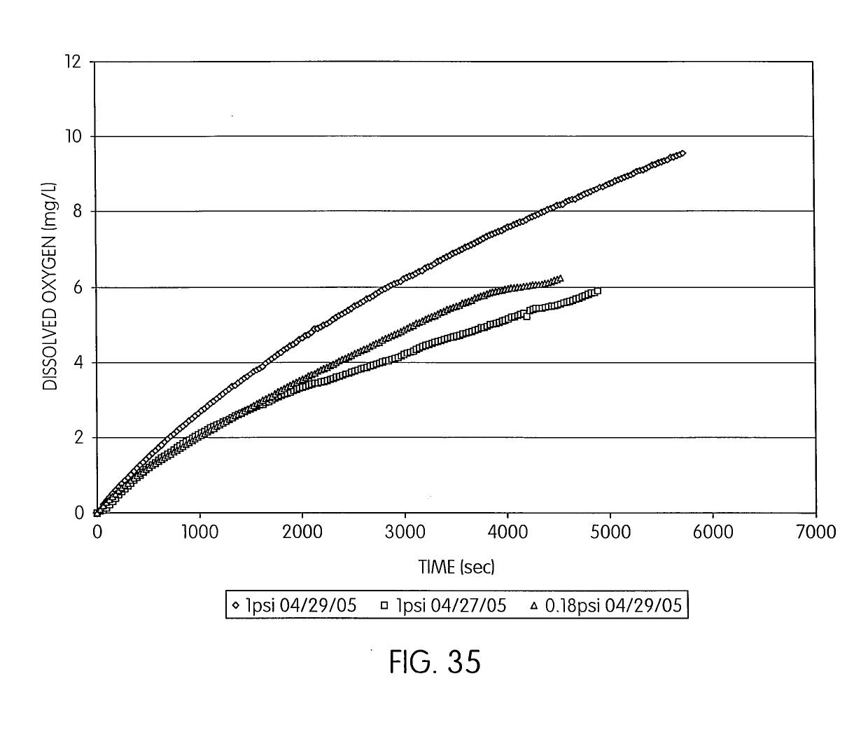

The water was deoxygenated by purging with nitrogen (N₂) until the sensor reading was zero for oxygen, after which time the nitrogen source was disconnected. Oxygen was then introduced through the O₂ gas supply connected to the silicone catheter, and oxygen sensor readings were monitored. The output from the oxygen sensor is shown in FIG. 35 where the oxygen level was found to increase due to diffusion of oxygen through the silicone catheter wall into the water. From the initial slope of plot of oxygen concentration (y axis) over time (x axis), the flux of oxygen (in g/s) through the silicone catheter was determined. The oxygen flux measured experimentally was compared with the calculated (theoretical) initial oxygen flux according to Equation (3) below:

$$Flux ( g \text{/} s ) = \frac{P * A * Δ C}{L} \qquad ( 3 )$$

where
   P=Permeability (cm²s⁻¹)
   A=internal area catheter=π*ID*length (cm²)
   L=wall thickness of catheter (cm)
   ΔC=[Oxygen]_{catheter/water interface (saturation water 37° C.)}−[Oxygen]_{water (g/cm3)}
Att=0, [Oxygen]_{water}=0
   [Oxygen]_{catheter/water interface (saturation water 37° C.)}=31.6×10⁻⁶ g/cm³
   P=1.2×10⁻⁴ cm²s⁻¹

Silicone catheter parameters were substituted in Equation (3) above, resulting in calculated fluxₜ₌₀=5.0×10⁻⁷ g/s.

Experimental (actual) fluxes (g/s) were determined from initial slopes in FIG. 35 as: (1) 5.8×10⁻⁷; (2) 6.8×10⁻⁷ (repeat of Run (1) on a different date); and (3) 6.1×10⁻⁷. It should be noted that Run (3) had a lower O₂ supply pressure of 0.18 psi, whereas Runs (1) and (2) both had an O₂ supply pressure of 1 psi.

The experimental (actual) flux values were in reasonable agreement with the calculated flux value. The oxygen supply pressure did not appear to have a significant effect on the flux, as shown in Run (3). Thus, it was determined that the oxygen flux should be sufficient to meet islet oxygen requirements. For a device with 10,000 islets, 4 pmol/islet/min O₂ consumption, if a sealed silicone catheter is provided to meet or exceed all the oxygen needs of the islets, a flux of 2.13×10⁻⁸ g/s would be needed. For a device 1 cm long, the calculated flux (g/s) values of from (1)=5×10⁻⁸ g/s, this flux exceeds the islet oxygen requirements. (Kennedy et al., 1999, Biochem. Biophys. Res. Comm., 259:331-5 (4-11 pmolO₂/islet/min); Longo et al, 1991, J. Biol. Chem. 266:9314-9319 (6-14 pmol/islet/min); Sweet et al, 2002, Diabetes Technol. The. 4 (5) (4.5-5 pmoI/islet/min)

Existing battery cell oxygen technologies can meet islet oxygen requirements. For example, battery cells of ˜1.2 cm diameter of by 0.9 cm thickness could produce ˜160 cc O₂ total. A population of 10,000 islets would require approximately 1.3 cc O₂/day, such that one battery cell would meet the entire islet oxygen requirements for approximately 4 months.

### Example 3

### Formation of a PEG-Based Hydrogel and Encapsulation of Cells

Cells were encapsulated in a PEG-based hydrogel as described below. PEG having an average of 8 arms, at approximately 20,000 g/mol was functionalized with vinyl sulfone (VS) groups to form PEGVS solution, and further modified using a solution of RGD peptide, and crosslinked with dithiothreitol (DTT), to form a hydrogel having a nominal PEG concentration of approximately 10% (m/v), where the ratio of RGD peptide to VS was 50:1, and the ratio of thiol groups (SH) to VS groups was 1:1, in a total gel volume of 50 μl.

In a first step, pendant groups were added to vinyl-sulfone-functionalized PEG (PEG-VS). Twenty-five (25)μl of PEG-VS was centrifuged at 13000 rpm for 30 seconds, 6.15 μl RGD solution was added to PEGVS, and the mixture was rotary mixed (vortexed) for 3 seconds and incubated for 30 minutes at 37° C.

In a second step, cells were prepared for encapsulation. The required number of cells (suspending in growth or culture medium) were centrifuged under conditions sufficient to form a cell pellet. The majority of the supernatant (excess medium) was removed and the cell pellet was resuspended in the small amount of medium still present. Then, 6.35 μl of phosphate buffered saline (PBS) was added, and the cells were resuspended in PBS by pipetting (3×).

In the third step, functionalized PEG solution (PEG-VS-RGD) from the first step was combined with the approximately 6.35 μl volume of PBS-cell suspension from the second step and mixed by pipetting (3×).

Finally, 12.5 μl DTT solution was added to the PEG-VS-RGD-cell suspension and the solution was aspirated into a syringe with a needle of appropriate gauge and length, and then injected to a mold. The mixture was allowed to set (crosslink) at room temperature for about 5 minutes (the mixture usually sets within about 2-3 minutes). After this step, the cells were encapsulated in the PEG-based hydrogel having a shape determined by the mold.

TABLE 2
Solutions used in encapsulation of cells in PEG-based hydrogel

| | Stock | Vol | mass | Molecular | | | | |
|---|---|---|---|---|---|---|---|---|
| Solution | (mg/100 μl) | (μl) | (mg) | Mass (MM) | moles | f | mole f | Check |
| PEGVS | 20 | 25 | 5 | 20000 | 2.50E−07 | 8 | 2.00E−06 | |
| RGD | 0.667 | 6.15 | 0.041 | 1025 | 4.00E−08 | 1 | 4.00E−08 | 5.00E+01 |
| PBS | | 6.35 | | | | | | |
| DTT | 1.23 | 12.5 | 0.15375 | 154.01 | 9.98E−07 | 2 | 2.00E−06 | 9.98E−01 |

### Example 4

### Continuous Delivery of VEGF by an Osmotic Minipump

To study the effects of exposure to various growth factor levels over different time periods up to several months, recombinant VEGF was pumped for up to 10 days into a “neovascularization construct” that was subcutaneously implanted in rats, and blood vessel density, size, area, volume, and connectivity were measured.

A. Production of Construct

Polyurethane (PU) with well-defined open porosity (82% porosity, 157±1 μm pores) was produced by a variation on the phase inversion technique using pre-packed spherical microbeads as porogens. PU tubes were formed with an inner diameter of 2.5 mm, length of 12 mm and a wall-thickness of 0.5 mm. To allow tissue-ingrowth into the porous PU, but to eliminate ingrowth into the lumen, PU tubes were lined with 350 μm of expanded tetrafluoroethylene (ePTFE) (Zeus, Orangeburg, S.C., USA), producing a structure similar to container 22 with cell barrier 24. A ePTFE plug (1.8 mm×3 mm) (Zeus, Orangeburg, S.C., USA) was attached to each end of the tube, one with a central hole of 1.2 mm diameter, where a 10 mm polyethylene catheter with an inner diameter of 0.9 mm was inserted (Scientific Commodities Inc., Lake Havasu, Ariz., USA). Finally, osmotic mini pumps of different sizes were attached, as described below, to produce a total of 56 “neovascularisation constructs,” also referred to as “vascularization constructs,” “constructs,” or “vascularization assemblies.”

Recombinant VEGF₁₆₅ (hereinafter, “VEGF”) was produced using standard recombinant DNA technology resulting in a concentration of 250 μg/ml. Osmotic mini pumps were purchased from Alzet®. (Durect Corporation, Cupertino, Calif., USA). To achieve continuous delivery of VEGF for at least 10 days, Alzet® model “1002” having a pump rate of 0.22 μl/h, a maximum pumping duration of 14 days and a filling volume of 108 μl was chosen. Pumps were filled with either with VEGF (250 μg/ml) or an equal amount of PBS (Gibco, Paisley, UK) (n=8) using the sterile filling needle provided by the manufacturer. According to themanufacturer's protocol, pumps were incubated in Nanopure® water (Barnstead International Dubuque, Iowa, USA) at 37° C. overnight. After sterilization in 70% ethanol (Sigma, Steinheim, Germany) for 12 hours, the ePTFE-lined PU tubes were permeabilized subsequently by flushing with 10 ml of 97% ethanol (Sigma, Steinheim, Germany) followed by 10 ml of PBS. PU tubes were filled with 20 μl of VEGF or PBS, respectively, and attached to the pumps via a flexible 10 mm long polyethylene catheter (0.9 mm inner diameter) (Scientific Commodities Inc., Lake Havasu, Ariz., USA), which was pre-filled with 8 μl of VEGF or PBS, respectively. Pump-tube constructs were kept in 37° C. PBS until subcutaneous implantation on the same day. To verify PBS and VEGF delivery at explantation, osmotic pumps were compressed manually to complete emptiness. The rest volume was measured and mean pump rates were calculated.

B. Subcutaneous Implantation of Constructs

Male Wistar rats (234 g-254 g body weight) were anaesthetized with a standard anesthetic mixture of 10% ketamine hydrochloride (Anaket-V™, Centaur Labs, Bayer AG, Germany) plus 2% xylazine (Rompun™ Bayer AG, Germany), (ratio 9 to 5 v/v) i.m. (approx. 0.05-0.1 ml/100 g BW). A longitudinal skin incision of one centimeter length was made on each side in the dorsal paramedian skin to create a subcutaneous pouch large enough for one vascularization assembly to be implanted on each side of each rat. Care was taken not to bend or kink the flexible polyethylene catheter between the osmotic pump and the polyurethane tube. The different groups of constructs were implanted using a rotational algorithm to ensure an equal distribution of groups at each implant position. Each rat received one construct delivering PBS and one construct delivering VEGF to the polyurethane tube. In total, 56 ePTFE-lined constructs as well as 6 non-lined constructs were implanted. The wounds were subsequently closed by placing two single stitches of prolene 4-0. For postsurgical analgesia, buprenorphine (Temgesic®, Bayer AG, Germany) was injected i.m. (approx. 0.1 ml/70 g BW) in the back leg after the animal started to recover from anesthesia. Constructs were implanted for 10 days. Rats were kept in standard cages and had access to a commercially fabricated pelletted diet and water ad libitum. At the end of the implantation periods, rats were killed with CO₂ gas and constructs were explanted. All animal experiments were approved by the “University of Cape Town's Animal Research Ethics Committees”.

C. Histological and Image Analysis of Implanted Constructs.

Pumps still attached to the polyethylene catheter were separated from the polyurethane tubes, which were cut in half and fixed in 4% formalin for H&E and 10% zinc fixative for CD31 staining, respectively. Images of H & E and CD31-stained cross sections were taken at a 100× magnification using a digital microscope (Nikon Cool Scope, Nikon, Japan). To analyze a complete cross section of the construct, the microscope took between 45 and 60 single frames at 100× magnification in an automated fashion and stitched the separate frames together without overlap by the use of “Eclipse-net” software (Nikon, Japan). Tissue ingrowth was assessed on H & E cross sections and expressed as percentage tissue ingrowth of the porous space within the polyurethane. Furthermore, in constructs without ePTFE lining, the area of tissue growing into the lumen of the PU construct was assessed and expressed as percentage of the total luminal area. Vessels were counted semi-automatically using the Visiopharm® image analysis program (Visiopharm® Integrator System, Copenhagen, Denmark) Vessels were defined as CD31 positive structures within the area of interest, that either show a transparent lumen or erythrocytes within. The area of interest was defined as the porous area within the polyurethane. Vascular density was reported as vessels/mm2. Because neovascularisation created by VEGF165 appeared to be irregular in shape on histological cross sections lacking typically rounded, clearly defined shapes, vascular area rather than vessel density was used to quantify neovascularisation from the second study onwards. Vascular area was defined as vessel area per tissue ingrowth area and was quantified as % IAV (ingrowth area vascularized). Furthermore, the mean vascular diameter was determined and reported in μm.

All data given on vessel density and vascular area was expressed as mean values±standard error of mean. Differences between groups were compared by students two-tailed T-test. A value of p<0.05 was considered significant.

Results Constructs consisting of an osmotic mini-pump, the polyethylene catheter and the polyurethane tube lined on the inside with ePTFE, as well as 6 non ePTFE lined constructs, were implanted into subcutaneous pockets of 6 Wistar rats. The surgical approach was relatively simple and all animals gained weight appropriately without signs of discomfort or behavioral abnormalities (321.5±9.18 g after 10 days vs. 249.67±6.46 g at implantation date, p<0.05). At explantation, all constructs have been found connected and macroscopically functional without signs of kinking in any parts. Tissue reaction in terms of vascularisation and capsule formation appeared to be localized within approximately 5 mm around the polyurethane tube. After 10 days of subcutaneous implantation, measured osmotic pump rest volume, as described in chapter 6.2.2, was 53.67±2.08 μl in the PBS group, 51.67±2.89 μl in the ePTFE lined PBS group, 57.67±3.05 μl in the VEGF group and 55±4.58 μl in the ePTFE lined VEGF group. Differences between groups were not statistically significant. Mean calculated pump rates were 0.226±0.01 μl/h for the PBS filled pumps, 0.235±0.01 μl/h for the ePTFE lined PBS filled pumps, 0.21±0.01 μl/h for the VEGF-filled pumps and 0.221±0.02 μl/h for the ePTFE lined VEGF-filled pumps.

Image analysis showed a 100% tissue ingrowth into the pores of the PU construct after 10 days in all four different groups studied. Tissue ingrowth into the luminal space of PBS filled, non-lined constructs was 64.12±13.81% on average after 10 days of subcutaneous implantation and almost complete in some samples. Luminal lining with ePTFE prevented tissue ingrowth at the inner surface of the PU effectively and only allowed minimal ingrowth into the lumen of PBS delivered constructs (1.7±1.7% tissue ingrowth) (p<0.05). VEGF-filled, non lined constructs showed a tissue ingrowth of 60.66±12.65%, which was reduced to 1.8±1.8% for the VEGF-filled, ePTFE lined constructs (p<0.05). Since prevention of intraluminal tissue ingrowth represented a prerequisite for further studies involving growth factor delivery for extended time periods, all constructs used after the initial study were lined with ePTFE.

Vessel density increased significantly after 10 days of continuous VEGF (250 μg/ml, 1 μg/day) by 75.19% (146.9±24.6 vessels/mm² for VEGF-pumped vs. 83.8±8.3 vessels/mm² for PBS pumped constructs, p<0.05). In parallel, vascular area increased significantly by 154.21% (9.57±1.7% IAV for VEGF-pumped constructs vs. 3.76±0.2% IAV for PBS pumped constructs, p<0.05) by continuous delivery of VEGF for 10 days.

Vessel density constructs pumped with PBS vs. VEGF (250 μg/ml, 1 μg/day) shows significantly increased vessel density of VEGF-delivered constructs compared to PBS-delivered constructs (146.9±24.6 vessels/mm² vs. 83.8±8.3 vessels/mm2, p<0.05).

The effect of VEGF dosage was evaluated by delivery of VEGF (Alzet® osmotic mini pumps, model 1002, 0.22 μl/h pump rate, 14 days, 108 μl volume) at 2.5 μg/ml (250 μg/ml diluted 1:100 in PBS), which equals 10 ng VEGF per day. 25 μg/ml (250 μg/ml diluted 1:10 in PBS), which equals 100 ng VEGF per day and 250 μg/ml, which equals 1 μg VEGF per day (n=4). PBS, which was also pumped for 10 days, served as control (n=4). Results indicated that 10 days of continuous PBS delivery induced a vascular area of 2.4±0.13% IAV within the porous polyurethane of the construct. VEGF at 10 ng/day produced no increase in vascular area (2.11±0.56% IAV). VEGF at 100 ng/day and 165 1 μg/day, in contrast, increased vascular area by 144.9% (5.88±0.73% IAV) and 265.1% (8.76±1.37% IAV) compared to PBS, respectively (p<0.05 in both cases) No significant difference was seen between medium and high VEGF dose (p=0.11).

D. Quantification of Functional Neovascularisation by Micro-CT, Lectin Perfusion, CD31 and Corrosion Casting

Lectin perfusion Prolonged VEGF delivery to subcutaneously implanted constructs via osmotic mini pumps was carried out as described above. The circulatory systems of two rats were perfused with fluorescein isothiocyanate (FITC) labeled lycopersicon esculentum lectin (Vector Laboratories, Inc. Burlinghame, USA). For quantification of the functional neovascularisation into subcutaneously implanted porous polyurethane tubes, biotinylated lycopersicon esculentum lectin (Vector Laboratories, Inc. Burlinghame, USA) was used. Four (4) rats were perfused using a slight variation of a standard protocol, as described previously by Thurston et al. (Am J Physiol 271 (6 Pt 2):H2547-62.384, 1996). Briefly, 10 days after implantation, 500 μg of lycopersicon esculentum lectin was injected in 500 μl of 0.9% NaCl into the left femoral vein of anaesthetized rats (n=4). After the skin was incised over the left groin to visualize the left femoral vein, lectin was injected slowly using a 1 ml 29 Gauge single-use syringe with an integrated needle (Omnican 100, Braun, Melsungen, Germany) and a stereo-microscope at a magnification of 16 to 25 times (Leica MS 5, lightsource intralux 5000-1, vopi, Switzerland). After 3 minutes, the chest was opened via a midline sternotomy and the aortic lumen was entered via the left ventricular chamber using a 18 Gauge intravenous canula (Abbocath-T 18G×51 mm, Abbocath Ireland LTD). The right atrium was incised to allow for the exsanguination of the animal while the circulatory system was perfused with 100 ml 1% paraformaldehyde in PBS at 37° C. at 120 mmHg. The two pump-tube constructs in the back of each animal were explanted as well as the left kidney as a control for perfusion. Pumps still attached to the polyethylene catheter were separated from the polyurethane tubes, which were cut in half and fixed in 1% paraformaldehyde/PBS for the detection of biotinylated lectin (n=4) and 10% zinc fixative for CD31 staining (n=4) at room temperature for 24 hours. All samples were further processed and stained with anti-CD31 antibody using a standard protocol. For the detection of biotinylated lectin, 1% paraformaldehyde/PBS fixed, dewaxed sections were equilibrated in tris buffered saline (TBS) for 10 minutes. Streptavidin/alkaline phosphatase (K0610-11 DAKO, A/S, Glostrup, Denmark) was applied for 10 minutes at room temperature, rinsed in TBS and detected by applying BCIP/NBT for 8 minutes incubation. Samples were washed in water, dehydrated in increasing concentrations of alcohol and mounted in Entellan (Merck, Darmstadt, Germany).

Micro CT perfusion In order to visualize vascularization into the polyurethane tube by micro-CT, the vascular system of 4 rats was perfused with a radiopaque contrast medium. To this end, 4 animals were injected with 700 I.U. heparin/kg body weight i.p. 30 minutes before perfusion. Immediately after the rats have been killed with CO2 gas, an abdominal midline incision was made and the descending aorta was exposed. A polyethylene catheter (PE 100 Intramedic Clay Adams, Becton Dickinson, USA, 0.86 mm I.D.) was inserted into the descending aorta, which has been tied off distally. After severing the inferior vena cava next to the aorta, the circulatory system was flushed with 60 ml of 0.9% saline at 37° C. containing 10 Units of heparin per ml of saline at a pressure of 120 mmHg. During this time, 10 ml of lead chromate containing radiopaque silicone rubber (Microfil MV-122, Flow Tech; Carver, Mass., USA) was prepared and infused immediately afterwards at a pressure of 120 mmHg into the abdominal aorta “retrograde” following the saline perfusion. Once the silicone rubber started to gel, the infusion was stopped and allowed to further solidify for 1 hour until explantation of the subcutaneous vascularization implants. Implants were stored at 4° C. in 10% neutral buffered formalin until they were scanned.

Micro-CT imaging Neovascularisation in the VEGF or PBS perfused polyurethane tubes of the constructs was imaged using a commercially available, high resolution (6 μm nominal resolution) desktop micro-CT scanner (μ-CT 40, Scanco Medical AG, Bassersdorf, Switzerland). he scanning time for 1000 sections of each sample was 288 minutes at an energy of 55 kVp and an intensity of 114 μA. To be able to analyse the fine vascular network including arterioles, capillaries and venules, we used the highest resolution of 6 μm, which created images of 2048×2048 pixels. A threshold of 220, which is the equivalent of 22% of the maximal grey scale value, was chosen to visualize the vessels in the specified area of interest. The area of interest was defined as the layer of polyurethane within the construct without the adjacent ePTFE on the inside and without incoming and outgoing vessels on the outside of the construct. In order to study the three dimensional vascular architecture, Feldkamp's cone-beam reconstruction algorithm was used (Feldkamp, J Opt Soc Am A 1:612-619, 1984). To quantify three-dimensional histomorphometric values such as vessel thickness, distribution of vessel thickness, vascular volume and connectivity, a method previously employed for the analysis of trabecular bone was used (Hildebrand et al., J Bone Miner Res 14(7):1167-74, 1999; Hildebrand et al. J Microsc 185:67-75, 1997).

Vessel thickness and distribution of vessel thickness was calculated using a method-independent of the model for assessing thickness in three dimensional images (Hildebrand et al. J Microsc 185:67-75, 1997). Vascular volume was calculated by computing voxel size and voxel number in 3D images after application of the threshold. Connectivity was defined as the number of interconnections within a network that can be devided before two separate networks are created (Odgaard and Gundersen, Bone 14(2):173-82, 1993). The semi automated software, which is provided with the micro-CT 40, is capable of generating histograms of the distribution of vessel sizes. Color coding highlights the micovasculature within the area of interest and subdivides different vessel diameters. Micro CT scanning as well as image analysis and calculations was provided by Mr. Marcus Burkhardt (Scanco Medical AG, Bassersdorf, Switzerland).

After micro-CT scanning, formalin fixed Microfil® perfused samples (n=4) were evaluated microscopically. Without alcohol dehydration, samples were embedded in wax and 10 μm thick sections were cut and mounted unstained on un-coated slides. Microfil® perfused microvessels were quantified using a digital automated microscope (Nikon Cool Scope, Nikon, Japan).

Corrosion casting perfusion Eight (8) constructs were subcutaneously implanted in 4 rats for 10 days, as described above. Standard methods were used for vascular corrosion casting (Lametschwandtner et al., In: Scan Electron Microsc pp. 663-95, 1984). Briefly, heparinised rats (700 I.U. heparin/kg body weight i.p.) were anaesthetized with a standard anesthetic mixture of ketamine hydrochloride 10% (Anaket-V™, Centaur Labs, Bayer AG, Germany) plus 2% xylazine (Rompun™ Bayer AG, Germany), (ratio 9 to 5 v/v) i.m. (approx. 0.05-0.1 ml/100 g BW). The chest was opened via a midline sternotomy and the aortic lumen was entered via the left ventricular chamber using a 18G i.v. canula (Abbocath-T 18G×51 mm, Abbocath Ireland LTD). The rats circulatory system was subsequently perfused with 100 ml phosphate buffered saline (PBS) at 37° C. at 120 mmHg after an incision in the right atrium was made to allow for exsanguination of the animal. Immediately afterwards, 15 ml of a mixture of 10/0.3 v/v Mercox Red/Catalyst (Ladd Research, Williston, Vt.) was infused at the same rate. After complete resin curing (1-2 days at room temperature), the two pump-tube constructs implanted at the back of each animal were explanted and soft tissue was macerated in 10% KOH (Sigma Aldrich, Steinheim, Germany) for 2-4 hours. Subsequently, casts inside and around the porous polyurethane were rinsed with hot water, dissected and air dried overnight.

Scanning Electron Microscopy analysis of corrosion casts Dry samples consisting of the casting material and the PU tubes lined with ePTFE were cut in half, mounted on metal stubs using double-sided gluing tape and colloidal silver to achieve conductivity. Samples were further sputter coated with gold (Polaron Range SC 7640 Sputter Coater) at 1.5 kV for 3 minutes and examined using a Scanning Electron Microscope (Jeol SEM, JSM 5200, Japan) at 15, 35, 50, 100, 150, 200, 350, 500, 750 and 1000 times magnification. Images were captured by the use of “Orion 5 for Windows” control systems high resolution image software (Jeol, Japan). After images were taken of the outside of the construct, 5 mm cross sections were cut, mounted, sputter coated, and scanned again. Image analysis of vascular area inside the polyurethane pores was carried out using “Leica Q Win for Windows” (Leica, Wetzlar, Germany), whereby the whole area of a 5 mm cross section (12 frames at 150× magnification) was analyzed. Total vascular area of a cross section was reported as μm2.

For initial experiments to evaluate lectin perfusion of tracheal capillary networks of rats, fluorescein isothiocyanate (FITC) labeled leucopersicon esculentum lectin was detected by UV light using a Fluorescent Microscope (Nikon Eclipse 90i, Nikon, Japan) equipped with a fluorescent illumination system (Nikon X-Cite™ 120, Nikon, Japan) attached to a digital camera (Nikon DXM 1200 C, Nikon, Japan). Images of biotinylated leucopersicon esculentum lectin, CD31 stained cross sections as well as unstained Microfil® perfused sections were taken at a 100× magnification using a digital automated microscope (Nikon Cool Scope, Nikon, Japan). To analyze a complete cross section of the construct, the microscope took between 45 and 60 single frames in an automated fashion and stitched the separate frames together without overlap by the use of “Eclipse-net” software (Nikon, Japan). Vessels were counted semiautomatically using the Visiopharm® image analysis program (Visiopharm® Integrator System, Kopenhagen, Denmark). Vessels were defined as lectin or CD31 positive structures within the area of interest, that either show a transparent lumen or erythrocytes within. The area of interest was defined as the porous area within the polyurethane. The outside of the construct was excluded from analysis, as was the luminal area of the construct and the ePTFE. Vascular density was reported as total vessels per cross section and vascular area was quantified as total vascular area in μm2 per cross section. Furthermore, the mean vascular diameter was calculated from the measured circumference divided by π and reported in μm, assuming roundness of vessels.

All data obtained on vessel sizes, distributions of vessel sizes, vascular areas, vascular volumes and connectivity is expressed as mean values±standard error of mean. Differences between groups were compared by two-tailed student's T-test. A value of p<0.05 was considered significant.

E. Results

Histo-morphometrical analysis of the implanted constructs via endothelial staining with anti CD31 antibody confirmed the strong angiogenic effect of VEGF on the construct, as described above. 3. Prolonged delivery of 1 μg/day VEGF (250 μg/ml) for 10 days showed an increase in vessel number, size and area in comparison to PBS treated controls. Mean vessel density increased significantly from 376.25±108.48 vessels/cross section to 820.5±92.34 vessels/cross section (increase of 118.07%, p<0.05). Mean vascular area increased from 67.12×103±14.37×103 μm2 to 216.37×103±37.28×103 μm2 (increase of 222.36%, p<0.05) and mean vessel diameter increased from 23.75±1.39 μm to 30.49±1.55 μm (increase of 28.38%, p<0.05).

In initial experiments, FITC-labeled lycopersicon esculentum lectin was successfully tested as intravital marker of perfused capillaries as demonstrated on tracheal vascular network of rats. In the same animals, neovascularisation into porous (PU) constructs after 10 day delivery of VEGF could be detected as well. To increase the detection sensitivity of perfused vascular networks, biotinylated leucopersicon esculentum lectin was used thereafter. Sustained VEGF delivery increased neovascularisation compared to PBS control significantly. Mean vessel density of lectin-positive stained vessels increased 7.3 fold from 53.25±3.64 vessels/cross section (PBS) to 389±51.73 vessels/cross section (VEGF, p<0.05). Vascuar area increased in parallel from 9.75×103±1.83×103 μm2 to 196.09×103±40.56×103 μm2, p<0.05). Overall, this translated to a 20.1 fold increase in vascular perfusion in VEGF-treated samples compared to PBS. Mean vessel diameter increased by 15.43%, as assessed by lectin perfusion from PBS to VEGF, and failed to reach the significance level (28.12±4.44 μm to 32.46±8.9 μm, p=n.s.) If the vessel density of lectin perfused vessels is compared to the CD31 positive stained vessels, the following becomes apparent: only 14.15% of CD31 positive stained vessels in the PBS group showed signs of lectin perfusion (53.25±3.64 vessels/cross section compared to 376.25±108.48 vessels/cross section)

Continuous delivery of VEGF for 10 days increased this “relative” vessel density to 47.41% (389±51.73 vessels/cross section vs. 820.5±92.34 vessels/cross section) Therefore, VEGF delivery not only increased absolute vessel density, but also seems to have increased perfusion above the expected level. Perfusion of kidneys as control for adequate systemic perfusion was positive in all samples, and showed no difference between PBS and VEGF groups.

To obtain a quasi three dimensional aspect of the vascular network created through continuous delivery of VEGF in comparison to PBS, the whole animal was “casted” using methyl methacrylate, the constructs were explanted, and surrounding soft tissue was corroded. Remaining vascular casts were analyzed quantitatively by scanning electron microscopy followed by image analysis, which confirmed the results obtained through lectin perfusion, and provided additional information about the spatial distribution of the microvascular network. Upon SEM analysis of corrosion casts, 10 days of continuous PBS delivery via osmotic mini pumps into the construct created 55.75±10.51 vessels/cross section. VEGF for 10 days increased the vessel density 4.13 fold (230.5±30.17 vessels/cross section), (p<0.05). Vascular area increased 10.42 fold (22.06×103±7.1×103 μm2 vs. 229.7×103±19.54×103 μm2, p<0.05) and vessel diameter increased 1.91 fold (16.58±0.75 vs. 31.73±1.87 μm, p<0.05).

In vitro experiments involving micro-CT scanning of the vascularisation construct showed a threshold of 60 (6% of maximal grey scale value) for polyurethane and a threshold of 90 (9% of maximal grey scale value) for ePTFE. For in vivo experiments, the threshold was set to 220 (22% of maximal grey scale value) for the measurements of vessels which were filled with lead chromate containing silicone rubber (Microfil MV-122, Flow Tech; Carver, Mass., USA). Therefore, vessel measurement did not overlap or interfere with false positive detection of polyurethane.

New vessels grown into the PU of construct, which has a planimetric pore size of 157±1 μm, were detected by micro-CT scanning between 6 μm and 144 μm vessel size. Micro CT determined a vessel density of 0.93±0.1 vessels/mm in PBS perfused constructs. VEGF for 10 days increased vessel density 3.64 fold (3.37±0.58 vessels/mm, p<0.05). Vascular volume increased 17.38 fold from 0.16±0.06 mm3 (PBS) to 2.77±0.67 mm3 (VEGF, p<0.05). Average vessel diameter increased by 1.47 fold from 44.5±1.73 μm (PBS) to 65.6±4.03 μm (VEGF, p<0.05). Due to the 3-dimensional quantification capabilities, μ-CT also provided vessel connectivity data. Mean vessel connectivity increased significantly from 0.3±0.074/mm3 (PBS) to 62.34±18.75/mm3 (VEGF, p<0.05). Apart from increases in vessel number, vascular volume, diameters and connectivity, image analysis indicated an even distribution of neovascularisation throughout the whole porous construct in the VEGF-treated group. The PBS treated constructs, however, were less vascularized and the distribution of neovascularisation appeared to be uneven, including areas without any vascularisation apparent. Furthermore, analysis of the distribution of vessel sizes revealed, that VEGF-treated constructs showed a high percentage of 40 μm to 80 μm sized vessels reflecting growth of arterioles and venules, vessels of superior importance for a high level of blood flow.

After micro-CT scanning Microfil® filled constructs were examined histologically (n=4) to evaluate the capability of Microfil® to perfuse capillaries. Light microscopical analysis showed that capillaries as small as 7.07 μm were filled with Microfil®. Mean vessel density of PBS perfused constructs was 57.67±19.19 vessels/cross section and increased significantly by 2.4 fold, when VEGF165 was delivered for 10 days (139.67±19.68 vessels/cross section), (p<0.05). Mean vessel size increased by 1.78 fold from 23.34±5.67 μm to 41.55±9.63 μm (p<0.05). The same vessels measured histologically had therefore 1.91 fold (PBS) and 1.58 fold (VEGF) smaller diameters compared to micro-CT determined vessel diameters, but the relative increase induced by VEGF in comparison to PBS was similar (1.47 fold by micro-CT and 1.78 fold by histology), suggesting that, if histological measurements are considered to be relatively correct, then micro-CT may have overestimated microvessel size by 1.5 to 2 fold. In parallel, increases in vessel density were 3.64 fold by micro-CT and 2.4 fold as determined by histological evaluation.

While specific embodiments and examples have been described above, it will be appreciated that the invention may be practiced otherwise than as described. The descriptions above are intended to be illustrative and not limiting. Thus it will be apparent to one skilled in the art that modifications may be made to the invention as described without departing from the scope of the claims set out below.

1-90. (canceled)

91. An assembly for providing a vascularized, implanted device, comprising:
an implantable container having an exterior wall portion and an interior wall portion, said exterior wall portion being permeable to materials of a greater size than said interior wall portion;
a source connected with the implantable container that provides growth factors to the container; and
said interior wall portion enabling the growth factors to pass therethrough to reach the exterior wall portion to facilitate vessel formation on the exterior wall portion, said interior wall portion preventing substantial vessel formation within confines defined thereby.

92-95. (canceled)

96. An implantable device for providing therapy to a living body, comprising:
a container permeable to growth factors;
an agent source for providing at least one agent to the container, the agent source arranged to be in communication with the container through a conduit;
wherein a surface of at least one of the agent source, container, or conduit is coated with a growth factor composition.

97. The device of claim 96, wherein at least one growth factor is eluted from the surface coated with a growth factor composition and emitted to the body.

98. The device of claim 96, further comprising therapeutic living cells carried by the container, said living cells capable of generating cell products that are emitted from the permeable container to the living body.

99. The device of claim 98, further wherein the agent source provides at least one agent to the living cells carried by the container, wherein the agent enhances living conditions for cells.

100. The device of claim 97, wherein the surface is coated with a growth factor composition comprising VEGF, and VEGF is eluted from the surface and emitted to the body.

101. The device of claim 97, wherein the surface is coated with APGS and at least one growth factor is eluted from the surface and emitted to the body.

102. An implantable device for providing therapy to a living body, comprising:
a container permeable to growth factors;
an agent source for providing at least one agent to the container, the agent source arranged to be in communication with the container through a conduit, wherein a fluid stream passes through the container, the agent source, and the conduit; and
a polymer system in contact with the fluid stream, wherein the polymer system comprises a growth factor composition formed as part of the polymer system, so as to allow at least one growth factor to be eluted into the fluid stream;
wherein the polymer system is placed in the device to be in contact with the fluid stream.

103. The device of claim 102, wherein at least one growth factor is eluted from the polymer system and emitted to the body.

104. The device of claim 102, further comprising therapeutic living cells carried by the container, said living cells capable of generating cell products that are emitted from the permeable container to the living body.

105. The device of claim 104, further wherein the agent source provides at least one agent to the living cells carried by the container, wherein the agent enhances living conditions for cells.

106. The device of claim 103, wherein the polymer system comprises a growth factor composition comprising VEGF, and VEGF is eluted from the polymer system and emitted to the body.

107. The device of claim 103, wherein the polymer system comprises APGS and at least one growth factor is eluted from the polymer system and emitted to the body.
